(19) United States
(12) Patent Application Publication
(10) Pub. No.: US 2024/0229020 A9
(43) Pub. Date: Jul. 11, 2024

(54) Structured Nucleic Acid Templated Architectures

(71) Applicant: Massachusetts Institute of Technology, Cambridge, MA (US)

(72) Inventors: Nayan Agarwal, Cambridge, MA (US); Ashwin Gopinath, Cambridge, MA (US); Emily Wu, Cambridge, MA (US)

(21) Appl. No.: 18/343,768

(22) Filed: Jun. 29, 2023

Related U.S. Application Data
(60) Provisional application No. 63/497,734, filed on Apr. 23, 2023.
(60) Provisional application No. 63/367,322, filed on Jun. 29, 2022.

(51) Int. Cl. C12N 15/11; B01J 19/00

(52) U.S. Cl. CPC C12N 15/11; B01J 19/0046; B01J 2219/00722

(57) ABSTRACT

Provided herein are inorganic nucleic acid supramolecular structures and methods for making them. In certain aspects, the construct includes a structured nucleic acid polymer micelle, which micelle includes a structured nucleic acid template; one or more functional moieties attached to the template; and polymers that interact with nucleic acid present in the template to form the structured nucleic acid polymer micelle; and an inorganic shell surrounding the structured nucleic acid template, in which the one or more functional moieties extend outside the structured nucleic acid polymer micelle and the inorganic shell to maintain functionality.

### CROSS REFERENCE TO RELATED APPLICATIONS

This application claims the benefit of U.S. Provisional Patent Application No. 63/367,322, filed Jun. 29, 2022, and U.S. Provisional Patent Application No. 63/497,734, filed Apr. 24, 2023, both of which is incorporated by reference herein in their entirety.

### STATEMENT REGARDING FEDERALLY SPONSORED RESEARCH

This invention was made with government support under 031023-001 awarded by the National Science Foundation. The government has certain rights in the invention.

### REFERENCE TO AN ELECTRONIC SEQUENCE LISTING

The contents of the electronic sequence listing (22-1073-US_MIT25039_WO_ST26_Sequence_Listing.xml; Size: 2,715 bytes; and Date of Creation: Jun. 28, 2023) is herein incorporated by reference in its entirety.

### BACKGROUND OF THE DISCLOSURE

Nanomaterial manufacturing aims to efficiently create functional building blocks for complex higher-order assemblies. However, current fabrication techniques typically depend on lithography, which is subject to the constraints of Moore's law, resulting in increasing technically difficult and expense to create smaller and smaller structures.

In response, techniques that do not rely on lithography, and therefore Moore's law, have been developed. However, currently available techniques have difficulties, including insufficient control of building block formation and introduced randomness, resulting in less than optimally precise structures with less than optimally placed programmable surface functionalities.

For example, synthesis of inorganic (e.g., metal or metal-oxide) nanoparticles can produce particles with desirable material properties, such as stability over various environmental conditions, such as temperature, solution, pH, and solvents. But while synthesis conditions can be adapted to alter the material composition and size of the nanoparticles with relative ease, it is difficult to change the surface ligand composition, shape of the particle, to break the symmetry of the particle, or to tightly control the size of the particles while maintaining high monodispersity.

DNA origami have shown promise for fabrication of well-defined, nanometer-sized objects with selective addressability, i.e., control over placement of various materials within a DNA origami structure and on the surface of a DNA origami, with sub-nanometer precision. However, DNA structures can disassemble when removed from their assembly medium (typically an aqueous saline solution), and they have proved susceptible to high temperatures and extreme pH conditions.

Thus, there remains a need for nanomaterials that offer precise control of shape, size and composition, as well as precise control over location and composition of active moieties accessible on the surface of the nanomaterials, all while remaining amenable to varied solvent compositions and reaction conditions.

### SUMMARY

The present disclosure generally relates to functional nucleic acid supramolecular structure constructs and methods for making and using such constructs.

In one aspect, the disclosure provides a construct that includes a structured nucleic acid polymer micelle, which micelle includes a structured nucleic acid template; one or more functional moieties attached to the template; and polymers that interact with nucleic acid present in the template to form the structured nucleic acid polymer micelle; and an inorganic shell surrounding the structured nucleic acid template, in which the one or more functional moieties extend outside the structured nucleic acid polymer micelle and the inorganic shell.

In some embodiments, the construct includes one or more functional moieties that include an oligonucleotide that is complimentary to single-stranded nucleic acid extensions placed at one or more locations on the DNAO, and one or more functional moiety that include a co-polymer, the co-polymer including a cationic polymer that electrostatically binds to negatively charged DNAO, and a nonionic amphiphilic polymer. In some embodiments, one or more functional moieties further include a functional terminal group. In some embodiments, a DNA template is bound to a first functional terminal group on one or more functional moiety that comprises an oligonucleotide that is complimentary to single-stranded nucleic acid extensions placed at one or more locations on the DNAO, and a primer pair is bound to a functional terminal group on the one or more co-polymer functional moieties.

In some embodiments, the construct is bound to a surface through a second functional terminal group on one or more functional moiety that comprises an oligonucleotide that is complimentary to single-stranded nucleic acid extensions placed at one or more locations on the DNAO.

In another aspect, a composition is provided that includes a plurality of functional nucleic acid supramolecular structure constructs.

In another aspect, a method for making a functional nucleic acid supramolecular structure construct is provided, including providing a structured nucleic acid template having one or more functional moiety attached to the template; binding the structured nucleic acid template with polymers that interact with nucleic acid present in the template to form a structured nucleic acid polymer micelle; and encasing the structured nucleic acid template in an inorganic shell.

In some embodiments of the method, the one or more functional moiety attached to the template include (i) one or more oligonucleotide complimentary to single-stranded nucleic acid extension(s) placed at one or more locations on the structured nucleic acid template, (ii) a polymer spacer, and (iii) a functional terminal group; and co-polymers that bind the structured nucleic acid template include (i) a cationic polymer that electrostatically binds to negatively charged structured nucleic acid template, (ii) a nonionic amphiphilic polymer; and (iii) a functional terminal group bound to the nonionic amphiphilic polymer opposite to the bound cationic polymer that interact with nucleic acid present in the structured nucleic acid template to form a structured nucleic acid polymer micelle; and the method further includes binding at least one DNA template to a functional terminal group bound to a functional moiety complimentary to single-stranded nucleic acid extensions placed at one or more locations on the structured nucleic acid template; and binding at least one primer pair to a functional terminal group bound to a functional moiety comprising a co-polymer.

In some embodiments, the method further incudes binding the construct to a surface through a second functional terminal group on one or more functional moiety that comprises an oligonucleotide that is complimentary to single-stranded nucleic acid extensions placed at one or more locations on the structured nucleic acid template, in which the second functional terminal group is part of a functional moiety distinct from the functional moiety that binds to at least one DNA template. In some embodiments, the surface includes a flow cell.

In another aspect, the disclosure provides a monofunctional DNA origami nanoparticle conjugate construct, including: (a) an inorganic core; (b) one or more structured nucleic acid (DNAO) template attached to the core; (c) a binding functional moiety attached to the DNAO template for binding to a target analyte; and (d) one or more detection functional moiety attached to the DNAO template for detection of binding of a target analyte by the binding functional moiety.

In another aspect, the disclosure provides a monofunctional DNA origami nanoparticle conjugate construct that includes (a) an inorganic core; (b) one or more structured nucleic acid (DNAO) template attached to the core; (c) a binding functional moiety attached to the DNAO template for binding to a target analyte; and (d) one or more detection functional moiety attached to the DNAO template for detection of binding of a target analyte by the binding functional moiety, wherein the DNAO further includes (i) polymers that interact with nucleic acid present in the DNAO template to form a structured nucleic acid polymer micelle (DOPM), and (ii) an inorganic shell surrounding the DOPM (DOPM-NP), in which the binding functional moiety and detection functional moiety extend outside the DOPM and the inorganic shell.

In another aspect, a method for creating a monoclonal cluster of a target oligonucleotides on a particle is provided, including (a) providing a construct of the various aspects and embodiments described herein, (b) binding a DNA template to a first functional terminal group on one or more functional moiety that that binds to single-stranded nucleic acid extensions placed at one or more locations on the structured nucleic acid template, (c) binding a primer pair to a functional terminal group on the one or more co-polymer functional moiety, wherein the primer pair comprises a first primer sequence and a second primer sequence, the first primer sequence being complementary to either a forward strand or a reverse strand of the DNA template, and the second primer sequence being complementary to a DNA strand opposite the first primer sequence; and one primer in the primer pair comprises a cleavable linker, (d) amplifying the DNA template on the surface of the construct by bridge amplification, and (e) cleaving the cleavable linker. In embodiments, a high-quality monoclonal polymerase colony (Polony) for use in DNA sequencing is provided.

In some embodiments, the method is performed in solution, and in some embodiments, the method is performed with the construct immobilized to a surface, which includes, in some embodiments, a surface of a patterned or unpatterned flow cell.

### BRIEF DESCRIPTION OF THE DRAWINGS

FIG. 1 illustrates the strategy for using DNA origami (DNAO) as a template for inorganic material growth. Negatively charged DNAO is designed to carry a single functional ligand (dark wavy line with a circle at one end), which is made salt- and solution-independent using the polyplex micellization strategy (A), where cationic PEG-PLys block copolymers bind to DNAO via electrostatic interactions to form DNA origami polyplex micelles (DOPMs). These monofunctional-DOPMs are used as templates for silica growth using tetramethyl orthosilicate (TMOS) as precursor. Using this strategy, the monofunctionality is maintained while producing silica nanoparticles.

FIG. 2A shows the effect of different buffer conditions on the 42HB DOPMs (tubes 2 to 5; the blue rectangular structures depict 42HBs with the PEG chains in green). Tube 1 represents the 42HB DNAO in native buffer. The structural stability and dispersity across the different solvent conditions (depicted in grey background for native buffer, blue for water and yellow for 85% ethanol) was analyzed using DLS. FIG. 2B shows a preliminary test to grow silica shell on 42HB DOPMs in 85% ethanol at pH 11 using tetraethyl orthosilicate (TEOS) as precursor. The silica growth on 42HBs was observed using DLS (Dynamic light scattering) for an incubation period of 30 min. The upper and lower curves represent the measured hydrodynamic diameter (in nm) and scattering intensity curves (in counts/s) as a function of the growth time, respectively.

FIG. 3 shows a graph of DLS measurements of silica growth without the presence of DNA origami as a template (lower curve at longer times). Changes in the Z_{avg} were plotted as a function of the incubation time. As a comparison the silica growth with DNA origami as a template is overlayed in the graph (upper curve at longer times). For this, the growth was conducted in 85% ethanol, pH 11 reaction solution using TEOS as a precursor at 1× concentration. The growth reaction with origami was performed with DNA brick polyplexed with PEG-PLL, PEG length 5 kDa.

FIG. 4A to FIG. 4N collectively show DNA brick origami structures that are subjected to various conditions that impact the silica shell growth to obtain fine-tuned control over the growth process. FIG. 4A-FIG. 4D show DLS data for the optimization tests for solvent pH conditions. FIG. 4E-FIG. 4H show DLS data for alcohol-to-water content optimizations. FIG. 4I-FIG. 4L show DLS data for optimizing the choice of precursor (molecular structure of the tested precursors in FIG. 4I) and precursor concentrations. FIG. 4M-FIG. 4N show DLS and TEM data for PEG-length optimization studies. Scale bars=100 nm.

FIG. 5A and FIG. 5B show graphs of DLS measurements of pH transition from 11 to 7 using HCl during the silica growth for DOPMs with 5 kDa PEG chain length at 1× (FIG. 5A) or 10× (FIG. 5B) TEOS concentration. Changes in the Zavg were plotted as a function of the incubation time. For this, the growth was conducted in 85% ethanol, pH 11 reaction solution. The growth reaction was performed with DNA brick polyplexed with PEG-PLL, PEG length 5 kDa.

FIG. 6 shows a graph of DLS measurements of silica growth in methanol for DOPMs with 5 kDa PEG chain length using either TEOS or TMOS as precursor (longer curve for 10×TEOS and shorter curve for 125×TMOS concentration). Changes in the Z_{avg} were plotted as a function of the incubation time. For this, the growth was conducted in 85% methanol, pH 11 reaction solution. The growth reaction was performed with DNA brick polyplexed with PEG-PLL, PEG length 5 kDa.

FIG. 7 shows a graph of DLS measurements of the DOPMs with varying lengths of the PEG chain in different buffer conditions. Three variations of PEG-PLL were used with a constant cationic segment of 10 lysine repeats while using either of the lengths for the PEG chain: 1 kDa (left bar in each group), 5 kDa (middle bar in each group) or 20 (right bar in each group) kDa. For 5 and 20 kDa PEG lengths, the Zavg average increased to a maximum for 85% ethanol buffer conditions (15% ultrapure water) with ˜90 nm for 5 kDa and ˜129 nm for 20 kDa PEG lengths. Further changes to pH 11 only had a slight decrease in the Zavg for either of the PEG lengths. The 1 kDa chains tended to form aggregates.

FIG. 8 shows a graph of DLS measurements of silica growth for DOPMs with varying PEG lengths (lower curve for 5 kDa and upper curve for 20 kDa PEG length). Changes in the Zavg were plotted as a function of the incubation time. For this, the growth was conducted in 85% ethanol, pH 11 reaction solution using TMOS as a precursor at 125× concentration. The growth reaction was performed with DNA brick.

FIG. 9A and FIG. 9B shows graphs of DLS and TEM data for growth reactions using varying TMOS concentrations that were conducted for 3 hours on 42HB DOPMs with 20 kDa PEG chain lengths.

FIG. 10A and FIG. 10B shows graphs of DLS measurements of silica growth for DOPMs with 20 kDa PEG chain length at varying TMOS concentration (125×, 150× and 200×TMOS concentration). For FIG. 10A changes in the derived mean count rate (counts/second) were plotted as a function of incubation time. For FIG. 10B changes in the Zavg were plotted as a function of the incubation time. The growth reaction was performed with DNA brick polyplexed with PEG-PLL, PEG length 20 kDa. FIG. 10C shows corresponding TEM images. Scale bars=50 nm.

FIG. 11A to FIG. 11E shows examples of size measurements of silica coated 42HBs using a semi-automated method on TEM images. FIG. 11A shows a small, selected region on the TEM image of silica coated 42HBs. FIG. 11B shows length and width profile (superimposed rectangles) around the structures of interest. FIG. 11C and FIG. 11D show 1st and 2nd derivative thresholding with baseline correction, respectively. FIG. 11E is a legend. The scale for all the TEM images=50 nm.

FIG. 12A to FIG. 12E show examples of size measurements of silica coated rings using a semi-automated method on TEM images. FIG. 12A shows a small, selected region on the TEM image of silica coated 42HBs. FIG. 12B shows length and width profile (thick blue lines with numbers) around the structures of interest. FIG. 12C and FIG. 12D show 1st and 2nd derivative thresholding with baseline correction, respectively. FIG. 12E is a legend. The scale for all the TEM images=50 nm.

FIG. 13A to FIG. 13C show graphs of DLS data for silica shell growth on 42HB DOPMs with 5 kDa PEG lengths using a 10× concentration of TEOS as the precursor. The silica shell growth in these set of reactions were terminated by the addition of PEG-Silane (PS). The PS was added to the reaction after 10 min growth. The addition steps are outlined in FIG. 13A. PS concentration was varied relative to the precursor concentration. FIG. 13B and FIG. 13C show Zavg and scattering plots, respectively.

FIG. 14A to FIG. 14C relate to DLS data for silica shell growth on 42HB DOPMs with 5 kDa PEG lengths using a 10× concentration of TEOS as the precursor. The silica shell growth in these set of reactions were terminated by the addition of HCl, enforcing the change of pH of the reaction mixture from pH 11 to 7. HCl was added at different time points to assess its effect. The steps are outlined in FIG. 14A, while FIG. 14B and FIG. 14C represent the Zavg and scattering plots, respectively.

FIG. 15A to FIG. 15C relate to DLS data for silica shell growth on 42HB DOPMs with 5 kDa PEG lengths using a 10× concentration of TEOS as the precursor. The silica shell growth in these set of reactions were terminated by the addition of PS and HCl. PS was added after 10 min incubation and followed the addition of HCl 2 min later. PS concentration was varied relative to the precursor concentration. The steps are outlined in FIG. 15A, while FIG. 15B and FIG. 15C represent the Zavg and scattering plots respectively.

FIG. 16A to FIG. 16E relate to DLS data for silica shell growth on 42HB DOPMs with 5 kDa PEG lengths using a 100× and 125× concentrations of TMOS as the precursor. The silica shell growth in these set of reactions were terminated by the addition of PEG-Silane (PS). The PS was added to the reaction after 5 or 15 min growth. The addition steps are outlined in FIG. 16A. PS concentration was varied relative to the precursor concentration. FIG. 16B, FIG. 16D and FIG. 16C, and FIG. 16E represent Zavg and scattering plots, respectively.

FIG. 17A to FIG. 17C relate to DLS data for silica shell growth on 42HB DOPMs with 5 kDa PEG lengths using a 125× concentration of TMOS as the precursor. The silica shell growth in these set of reactions were terminated by the addition of HCl, enforcing the change of pH of the reaction mixture from pH 11 to 7. HCl was added at different time points to assess its effect. The steps are outlined in FIG. 17A, while FIG. 17B and FIG. 17C represent the Zavg and scattering plots, respectively.

FIG. 18A and FIG. 18B relate to DLS measurements to understand the batch-to-batch impact on the aggregation time point. Changes in the Zavg were plotted as a function of the incubation time. For this, the growth was conducted in 85% ethanol, pH 11 reaction solution and using TEOS as the precursor. The growth reaction was performed with DNA brick polyplexed with PEG-PLL, PEG length 5 kDa. FIG. 18A: Batch-to-batch variations were compared with FIG. 18B showing some batch variations.

FIG. 19A to FIG. 19J relate to test structures for inorganic material growth using DNA origami as a template. FIG. 19A to FIG. 19E: 42HB DNA origami (DNA brick), FIG. 19F to FIG. 19J: Delftagon DNA origami (DNA ring). FIG. 19A and FIG. 19F show representative oxDNA models for the corresponding DNAO structures. TEM micrographs FIG. 19B and FIG. 19G are TEM micrographs of DNAO, FIG. 19C and FIG. 19H of DOPM, and FIG. 19D and FIG. 19I of silica-coated structures. For all the TEM images, Scale bars=100 nm and 50 nm for the inset. FIG. 19E and FIG. 19J are graphs comparing the width (for DNA brick) and the inner diameter (for DNA ring) of structures across all the variations as labeled on the x-axis.

FIG. 20 shows electron micrographs representing control DNA brick structures. Scale bars=100 nm (unless specified otherwise). For these structures, the TEM grids were stained with uranyl formate to facilitate superior contrast to observe the structures.

FIG. 21 shows electron micrographs representing polyplexed DNA brick structures. For micellization, the K10P20k block co-polymers were used. Scale bars=100 nm (unless specified otherwise). For these structures, the TEM grids were not stained with uranyl formate.

FIG. 22 shows electron micrographs representing silica coated DNA brick structures. For micellization, the K10P20k block co-polymers were used. For silica shell growth 125× concentration of TMOS was used. Scale bars=100 nm (unless specified otherwise). For these structures, the TEM grids were not stained with uranyl formate.

FIG. 23 shows electron micrographs representing silica coated DNA brick structures. For micellization, the K10P20k block co-polymers were used. For silica shell growth 150× concentration of TMOS was used. Scale bars=100 nm (unless specified otherwise). For these structures, the TEM grids were not stained with uranyl formate.

FIG. 24 shows electron micrographs representing silica coated DNA brick structures. For micellization, the K10P20k block co-polymers were used. For silica shell growth 200× concentration of TMOS was used. Scale bars=100 nm (unless specified otherwise). For these structures, the TEM grids were not stained with uranyl formate.

FIG. 25 shows electron micrographs of silica coated DNA brick structures. For micellization, the K10P20k block co-polymers were used. For silica shell growth 125× concentration of TMOS was used. The reaction was stopped using PEG-silane followed by the pH transition using HCl. Scale bars=100 nm (unless specified otherwise). For these structures, the TEM grids were not stained with uranyl formate.

FIG. 26 shows electron micrographs representing control DNA ring structures. Scale bars=100 nm (unless specified otherwise). For these structures, the TEM grids were stained with uranyl formate to facilitate superior contrast to observe the structures.

FIG. 27 shows electron micrographs representing polyplexed DNA ring structures. For micellization, the K10P20k block co-polymers were used. Scale bars=100 nm (unless specified otherwise). For these structures, the TEM grids were not stained with uranyl formate.

FIG. 28 shows Electron micrographs representing silica coated DNA ring structures. For micellization, the K10P20k block co-polymers were used. Scale bars=100 nm (unless specified otherwise). For these structures, the TEM grids were not stained with uranyl formate.

FIG. 29A to FIG. 29F relate to elemental analysis and plotted the energy dispersive spectra (EDS) for both block and ring nanoparticles. The electron micrographs compare the non-coated control DNA structures with the silica-coated ones (FIG. 29A and FIG. 29D; silica coated structures are black; whereas, non-coated structures are light gray). Scale bars=100 nm. FIG. 29B and FIG. 29C show Energy-Dispersive Spectroscopy (EDS) elemental analysis to confirm inorganic material growth of silica coated DNA brick structures and FIG. 29E and FIG. 29F show the same for silica coated DNA ring structures. FIG. 29B and FIG. 29E represent a collection of electron micrographs overlayed with elemental signal maps as mentioned in the top left of each image (Silica, Si; Oxygen, O; Nitrogen, N; Phosphorus, P). Scale bars=20 nm. C and F) Represent the energy-dispersive spectra against intensity, which has been correlated with the respective elements.

FIG. 30 shows electron micrographs representing silica coated DNA brick structures in comparison to the control non-polyplexed, non-silica coated structures (red arrows). For micellization, the K10P20k block co-polymers were used. Scale bars=100 nm (unless specified otherwise). For this, the TEM grids were not stained with uranyl formate.

FIG. 31 shows electron micrographs representing silica coated DNA ring structures in comparison to the control non-polyplexed, non-silica coated structures (red arrows). For micellization, the K10P20k block co-polymers were used. Scale bars=100 nm (unless specified otherwise). For this, the TEM grids were not stained with uranyl formate.

FIG. 32 shows Energy-Dispersive Spectroscopy (EDS) elemental analysis comparing the non-coated control DNA structures with the silica coated ones. The images represent a collection of electron micrographs overlayed with elemental signal maps as mentioned in the top left of each image (Silica=Si=‘yellow’; Oxygen=O=‘red’; Nitrogen=N=‘blue’; Phosphorus=P=‘green’). Scale bars=20 nm. In the bottom of the figure, the energy-dispersive spectra against intensity, which has been correlated with the respective elements for both DNAO and silica coated structures (the curve with higher peak for oxygen and peak for silicon).

FIG. 33A and FIG. 33B show agarose gel electrophoresis of the studies with thermal stability of structures.

FIG. 34 shows micrographs indicating thermostability of silica-coated structures and non-silica coated structures.

FIG. 35 shows AGE analysis to confirm the accessibility of functional moieties after silica growth.

FIG. 36 shows representative models for 42HB design. The top row graphics represent the perspective, top and the side view. The cadnano lattice view market with red circles represents the helices that consist of the binding sites. The top and bottom view marked with red dots represent the position of the 3 binding sites on the 42HB design that was used to attach IONPs.

FIG. 37A to FIG. 37C show the preparation of DBCO-PEG-oligonucleotide conjugate that serve as that binding site on the structures. FIG. 37A shows a schematic representation of the strategy that enables maintaining the functionality during silica growth.

FIG. 37B shows a schematic representation of the preparation of the conjugate. FIG. 37C shows a HPLC purification trace and the corresponding PAGE gel analysis of the different fractions that were collected after the purification.

FIG. 38A to FIG. 38C show attaching iron oxide nanoparticles (IONPs) to the functional silica DNA nanoparticle. FIG. 38A shows a schematic of the binding strategy. Biotin moieties placed at specific locations on the surface of the DNA nanoparticle were bound to streptavidin-functionalized 10 nm IONPs. FIG. 38B Binding statistics. C) TEM images representing the three variations of the binding sites. DNAO and DOPMs were used as controls for this study. Scale bars=50 nm.

FIG. 39 shows a collection of electron micrographs for DNA brick structures carrying a single binding site (monofunctional DNA nanoparticle). Scale bars=50 nm. For micellization, the K10P5k block co-polymers were used.

FIG. 40 shows a collection of electron micrographs for DNA brick structures carrying two binding sites. Scale bars=50 nm. For micellization, the K10P5k block co-polymers were used.

FIG. 41 shows a collection of electron micrographs for DNA brick structures carrying three binding sites. Scale bars=50 nm. For micellization, the K10P5k block co-polymers were used.

FIG. 42 shows DNA origami synthesis. The upper row illustrates the construction of single stranded DNA staple strands conjugated with a heterobifunctional polymer spacer carrying molecules O* and (terminal functional group) A. The lower row illustrates DNA origami synthesis utilizing single stranded DNA staple strands and conjugated single strand DNA staple strand(s), resulting in a structured nucleic acid with molecule ‘A’ attached, tethered to the origami and spatially separated from it by a polymer spacer. One or more functional molecules (or molecules with one or more functional terminal groupw) can be attached to a structured nucleic acid in this manner.

FIG. 43 shows stabilization of DNA origami: Step 1 involves the formation of a polyplex around the DNA origami using a diblock copolymer composed of charged and uncharged regions. The free terminal of the uncharged region also has a reactive molecule, ‘C’, for downstream processing. Step 2 involves crosslinking the diblock copolymer using an appropriate crosslinking agent to create a stable DNA origami. Step 3 involves growing a layer of silica using appropriate reagents and optimized material growth conditions to create an inorganic DNA nanoparticle. DNA origami not shown with an optional second functional molecule ‘B’.

FIG. 44 illustrates attaching primers onto the DNA nanoparticle by reacting Molecule ‘C’ on the particles with its complementary molecule C* on the primers' 3′ and 5′ ends. There is also a photocleavable linker between the C* molecule and the terminal end of one of the primers.

FIG. 45 shows the cross-sectional view of the DNA nanoparticles is shown along with the schema for capturing a DNA fragment and locally amplifying the same, in which step 1 involves incubating the ssDNA fragment with a molecule A* on its 5′ terminus in a denaturing environment and purification from unreacted library fragments; step 2 involves bridge amplification by thermocycling in the presence of polymerase and dNTPs; and step 3 involves photocleaving the photocleavable linker on one of the primers, which allows for its removal upon irradiation with light of appropriate wavelength.

FIG. 46 shows an alternate process for creating a structured nucleic acid polymer micelle that includes staple strands with functional terminal groups and a polymer spacer. Such micelles can be used to subsequently include an inorganic shell. DNA origami not shown with an optional second functional molecule ‘B’.

FIG. 47 illustrates organization of monoclonal clusters, formed in solution, on a flow cell including standard capture sites (e.g., P5 and P7). Addition of a capture strand that is partially complementary flow cell capture site(s) and partially complementary to a sequence (e.g., primer binding sequence) on the DNA nanoparticle can capture the construct comprising monoclonal cluster(s).

FIG. 48 illustrates binding and detection of a single molecule of a binding target (here, an antigen with two epitopes) with monofunctionalized DNAO-nanoparticle conjugates. The DNAO-NPs are incubated with the targets to capture them in solution, followed by rapid immobilization of DNAO-NP binding target complexes onto a surface functionalized with a secondary antibody via the application of a magnetic field. Excess DNAO-NPs are then washed away. Detection is then achieved by conducting, in this embodiment, fluorescence microscopy.

FIG. 49 illustrates binding and detection of a single molecule of a binding target (here, biotin) with monofunctionalized DNAO-nanoparticle conjugates. The DNAO-NPs are incubated with the targets to capture them in solution, followed by immobilization of DNAO-NP binding target complexes onto a surface functionalized with a secondary antibody (in this example, no magnetic field). Excess DNAO-NPs are then washed away. Detection is then achieved by conducting, in this embodiment, fluorescence microscopy.

FIG. 50A shows streptravidin congugated to Alexa Fluor 647, immobilized on a biotin-BSA surface. Imaged with 638 nm excitation, in liquid. FIG. 50B through FIG. 50G show 0, 100 fM, 300 fM, 1 pM, 3 pM, and 10 pM concentrations of fluorescent biotinylated DNAO-NPs immobilized on a biotin-BSA surface, respectively, imaged with 488 nm excitation, dry. FIG. 50H shows 10 pM fluorescent biotinylated DNAO-NPs immobilized on a biotin-BSA surface, to serve as a negative control.

FIG. 51 shows a dilution curve of the average number of counts derived from analyzing fluorescence microscopy images of biotinylated DNAO-NPs immobilized at concentrations between 0-10 pM on biotin-BSA. The number of images averaged for each concentration∈[10, 16]. The region of interest analyzed was 66.6 μm×66.6 μm.

FIG. 52A to FIG. 52C show various nanoflower structures; FIG. 52D and FIG. 52E show nanotaco structures.

FIG. 53A shows a nanoflower dimer, and FIG. 50B shows an agarose gel of nanoflower dimerization experiments under different conditions.

### DETAILED DESCRIPTION

Disclosed herein are functional nucleic acid supramolecular structures. The constructs of the disclosure are stable across a wide range of solvents that can be non-aqueous, salt-free as well as organic solvents; can sustain basic pH conditions as well as sudden pH transitions; can be designed to be thermostable and can survive at 90° C. in salt-free buffers; and the functionality of the nucleic acid supramolecular structures can be maintained after growth of an inorganic shell around a structured nucleic acid template core, allowing creation of monofunctional to multifunctional inorganic nanoparticles for quantification, therapeutic delivery, diagnostics in biology, genomics, proteomics, metabolomics where single-molecule studies for understanding molecular interactions is needed. Methods for making and using such constructs are also provided. Monofunctional DNA origami nanoparticle conjugates are also provided, as well as methods for making them.

A number of terms are introduced below:

The terms “DNAO”, “DNAO template”, “structured nucleic acid” or “structured nucleic acid template” refer to any structure built out of nucleic acids that has a well-defined shape and molecular weight. In some embodiments, a DNAO may comprise DNA, RNA, LNA, PNA, BNA or a combination thereof. The nucleic acids in the DNAO template may be homogeneous or heterogeneous (i.e.: all DNA; a combination of DNA and RNA and/or LNA, etc.). In some embodiments, DNAO can comprise DNA origami.

The terms “DOPM” or “structured nucleic acid polymer micelle” mean a structured nucleic acid template (i.e., DNAO) with one or more functional moieties attached to the template, and polymers that interact with nucleic acid present in the template to form a micelle-like structure with one or more functionalized DNAO surrounded by a layer of polymers.

The term “polyplex micelle” refers to a self-assembled complex formed using DNA origami and cationic block copolymers.

The term “DNA origami” refers to the folding single-stranded ‘scaffold’ DNA template molecules (also “scaffold strands”) into target structures at the nanoscale level, by annealing templates with of short ‘staple’ DNA strands (“staple strands”) through hybridization. DNA origami can be designed to fold into a desired shape, such as a brick, donut, triangle and the like.

The terms “about” and “approximately” mean plus or minus 10% of the recited measurement.

Structured Nucleic Acid-Based Nanomaterials

The disclosure details structured nucleic acid-based nanomaterials that exhibit significant thermostability and enzymatic stability, and methods of making such materials. In embodiments, constructs of the disclosure include monofunctional or polyfunctional inorganic nanoparticle structures, an example being an inorganic shell-covered structured nucleic acid polymer micelle. Such nanoparticles are programmable in nature due to the one or more functional moieties that can be precisely positioned on the nanoparticle surface, allowing them to organize other nanomaterials with high accuracy, e.g., binding biomolecules at one or more specific locations on the nanoparticle surface for detection or processing (e.g., sequencing) or serving as molecular building blocks to construct larger nanostructures. Such programmability can be useful for fields like biophysics, molecular biology, nanoelectronics, nanophotonic, and more.

In one aspect, a construct is provided that includes (i) a structured nucleic acid polymer micelle (DOPM), which DOPM includes a structured nucleic acid (DNAO) template; one or more functional moiety attached to the DNAO template; and polymers that interact with nucleic acid present in the DNAO template to form the DOPM; and (ii) an inorganic shell surrounding the DOPM, in which the one or more functional moiety extends outside the DOPM and the inorganic shell.

In embodiments, the DNAO template comprises DNA, RNA, LNA, or a combination thereof, and in embodiments, the construct can include more than one DNAO template. When two or more DNAO templates are present, in embodiments, the templates are bound together. In embodiments, a DNAO template can be formed from a single scaffold strand and staple strands, or more than one scaffold strand and staple strands. In certain embodiments, the DNAO template comprises a DNA origami template.

The construct includes polymers that interact with nucleic acid present in the DNAO template to form the DOPM. Any polymer that can interact with the nucleic acid present in the DNAO and permit growth of the inorganic shell on the DNAO may be used. In some embodiments, the polymer comprises a cationic polymer, a polymer that is complexed with cations, or a co-polymer that includes a cationic polymer. In certain embodiments, the polymers comprise co-polymers of: (a) a cationic segment that electrostatically binds to negatively charged DNAO, and (b) a nonionic amphiphilic polymer.

The negatively charged DNAO interacts with the cationic polymers in the DOPM, serves as the template for inorganic shell growth, and improves mechanical and chemical stability. The polymers may be bound over all or a portion of the DNAO. In one embodiment, the polymers comprise a layer surrounding the DNAO.

In some embodiments, the cationic polymers include, but are not limited to pLys, pHis, pArg, N,N-dimethylaminoethyl methacrylate (DMAEMA), poly amido amine (PAMAM), polyethyleneimine (PEI), cationic cyclodextrin, cationic cellulise, cationic dextrin, cationic dextran, chitosan, gelatin, spermine, spermidine, cationic surfactants such as CTAB or combination thereof.

In some embodiments, the nonionic amphiphilic polymers include, but are not limited to, polyethylene glycol (PEG), polyoxazolines (POX), PVP (poly(N-vinylpyrrolidone), polyglycerols (PG), or combinations thereof.

In some embodiments, the polymers include co-polymers of a cationic polymer that electrostatically binds to negatively charged DNAO, and PEG. In certain embodiments, the polymers comprise PEG-pLys, PEG-pArg, or PEG-pHis co-polymers, or combinations thereof.

The PEG monomers may be any molecular weight. In various embodiments, the PEG monomers are at least 2 kDa, 3 kDA, 4 kDa, or 5 kDa in molecular weight. In other embodiments, the PEG monomers are between 2 kDa and 100 kDa in molecular weight, between 3 kDa and 100 kDa in molecular weight, between 4 kDa and 100 kDa in molecular weight, or between 5 kDa and 100 kDa in molecular weight. In some embodiments, the PEG comprises PEG monomers of at least 2 KDa or at least 5 kDa in molecular weight.

Co-polymers for use in the constructs may comprise other segments to provide additional modularity. In non-limiting embodiments, there can be a thermosensitive segment (such as PNIPAM in a tri-block polymer setting such as: Plys-PNIPAM-PEG) that provides an option to modulate chain length of the polymeric shell as a function of temperature; and/or a photo-cleavable segment that can allow cleaving nonionic amphiphilic polymer chains by shining light and disabling the template for any material growth.

In embodiments, the polymers form a layer surrounding the DNAO. The polymer layer may be of any thickness as suitable for an intended use. In various embodiments, the polymer layer has a thickness of between about 1 nm and about 20 nm, between about 2.5 nm and about 20 nm, between about 5 nm and about 20 nm, between about 10 nm and about 20 nm, between about 5 nm and about 15 nm, between about 7.5 nm and about 20 nm, or between about 7.5 nm and about 15 nm. In some embodiments, the polymer layer has a thickness of between about 1 nm and about 20 nm.

Some embodiments that include a structured nucleic acid (DNAO) template; one or more functional moiety attached to the DNAO template; and polymers that interact with nucleic acid present in the DNAO template to form the DOPM, are polyplex micelles.

Constructs of this aspect of the disclosure include an inorganic shell surrounding the DNAO, such that the core nucleic acid structure of the DNAO (not including any functional moieties) is not accessible to the environment. The negatively charged DNAO interacts with the polymer, such as the cationic polymer, to produce the DOPM, which serves as the template for inorganic molecule growth. The inorganic molecule growth directly takes place on the surface of the combined DNAO-polymer structure (i.e., DPOM), in which the charged surface of the DNAO is neutralized by the cationic polymer. The outermost layer of such embodiments is any nonionic amphiphilic polymer component of the polymers and its terminal group (if any), with an inorganic shell layer below the nonionic amphiphilic polymer component layer, followed by the electrostatic complex of the DNAO and the cationic polymer. In some embodiments, the thickness of the inorganic shell is less than the length of the nonionic amphiphilic polymer component of the polymers, which allows it to extend beyond the surface of the inorganic shell and, e.g., be exposed to the surrounding environment (e.g., solvent, buffer, air). Any terminal group present can likewise be exposed.

Any inorganic material may be used as the shell component as suitable for an intended use of the construct. In various non-limiting embodiments, the inorganic shell comprises a silica shell (SiO₂), titania shell (TiO₂), alumina shell (Al₂O₃), gold shell, silver shell, copper shell, iron shell, platinum shell, palladium shell, calcium phosphate, hydroxyapatite shell, or combinations thereof. In some embodiments, the inorganic shell includes a silica shell, and in some embodiments.

The inorganic shell layer may be of any thickness as suitable for an intended use. In various embodiments, the inorganic shell layer has a thickness of between about 1 nm and about 20 nm, between about 2.5 nm and about 20 nm, between about 5 nm and about 20 nm, between about 10 nm and about 20 nm, between about 5 nm and about 15 nm, between about 7.5 nm and about 20 nm, or between about 7.5 nm and about 15 nm. In some embodiments, the inorganic shell is between 1 nm and 20 nm in thickness.

In embodiments, the DNAO is functionalized by attaching a one or more functional moiety at specific locations on the DNAO. The one or more functional moiety extends outside the DOPM and the inorganic shell, and thus is accessible to the environment. Thus, the DOPM includes one or more (i.e., 1, 2, 5, 10, 25, 50, 100, 250, 500, 1000, 2500, 500, 6500, 10000, and the like) functional moiety attached to the DNAO template. Any functional moiety may be used as appropriate for an intended use of the structure. In some embodiments, the one or more functional moiety may react or bind to other molecules or groups. In some embodiments, the one or more functional moiety is a single functional moiety (i.e., either a single functional moiety per DOPM, or multiple copies of the same functional moiety, or “monofunctional”). In other embodiments, the one or more functional moiety comprises two or more different functional moieties, each present in one or more copy, also “multifunctional” or “polyfunctional”).

In embodiments, the one or more functional moiety includes one or more polymer or polymer component, e.g., PEG. In embodiments, the functional moiety is made accessible after the inorganic material growth by using linear PEG chains. The PEG chains extend it further from the various layers of the final construct. The functional PEG chain may be any length so long as it extends outside the DOPM and inorganic shell. In one embodiment, the PEG monomers in the functional linear PEG chains are at least 100 kDa in molecular weight. In some embodiments, the polymer includes a cationic polymer or polymer component, and in embodiments, the functional moiety includes a co-polymer that itself includes (a) a cationic polymer that electrostatically binds to negatively charged DNAO, and (b) a nonionic amphiphilic polymer.

In some embodiments of the construct, the one or more functional moiety comprise an oligonucleotide that is complimentary to single-stranded nucleic acid extensions placed at one or more locations on the DNAO. In an embodiment, an oligonucleotide functional moiety is bound to a linear PEG functional moiety.

Some embodiments of the construct include one or more functional moiety that include an oligonucleotide that is complimentary to single-stranded nucleic acid extensions placed at one or more locations on the DNAO, and one or more functional moiety that include co-polymer comprising (a) a cationic polymer that electrostatically binds to negatively charged DNAO, and (b) a nonionic amphiphilic polymer.

In embodiments, the one or more functional moiety further include a functional terminal group, including but not limited to biotin, streptavidin, dibenzocyclooctyne group (DBCO), azide, alkyne, amine, NHS esters (N-hydroxysuccinimide esters), thiol, maleimide, iodoacetyl, carboxyl, and the like.

In some embodiments, the functional moiety may be bound to the polymer layer on the construct surface, such as via the nonionic amphiphilic polymer (including but not limited to the linear PEG discussed above). In other embodiments, the functional moiety (including but not limited to a detectable molecule, such as a fluorescent molecule) may be bound to an oligonucleotide extension bound to a linear PEG chain.

In some embodiments, the construct includes one or more functional moiety that includes (1) polymer bound to an oligonucleotide that is complimentary to single-stranded nucleic acid extensions placed at one or more locations on the DNAO, and a functional terminal group opposite the bound oligonucleotide, and/or (2) one or more co-polymer including (a) a cationic polymer that electrostatically binds to negatively charged DNAO, (b) a nonionic amphiphilic polymer, and (c) a functional terminal group bound to the nonionic amphiphilic polymer opposite to the bound cationic polymer.

In some embodiments, the functional terminal group (e.g., “C”—check figure) on the one or more co-polymers that electrostatically bind to the negatively charged DNAO is utilized to attach primer pairs onto the construct by reacting functional terminal group ‘C’ with its complementary molecule C* on the primers' 3′ and 5′ ends. In some embodiments, there is also a photocleavable linker between the C* molecule and the terminal end of one of the primers.

In some embodiments, the DNAO is stabilized by crosslinking, such as by bi-functional molecules that covalently react with DNA strands constituting the DNAO. For example, in embodiments that include PEG-PLys polymer, glutaraldehyde can be used to crosslink the lysine moieties on the PEG. In some embodiments, the construct is stable in non-aqueous, salt-free as well as organic solvents, and in some embodiments, the construct is stable in basic pH conditions and can sustain sudden pH changes, and in some embodiments, the construct is stable at 90° C. in salt-free buffer.

Some embodiments of the construct aspect and embodiments described above, further include a cargo embedded in the organic shell, including but not limited to a therapeutic, diagnostic, detectable moiety, and the like.

In another aspect, the disclosure provides a method for making a construct including (a) providing a DNAO template having one or more functional moiety attached to the DNAO; (b) binding the DNAO with polymers that interact with nucleic acid present in the DNAO template to form the DOPM; and (c) encasing the DNAO in an inorganic shell (with bound polymers present, i.e., a DOPM). In embodiments, the polymers can include those described above, including any polymer that can interact with the nucleic acid present in the DNAO and permit growth of the inorganic shell on the DNAO may be used. In some embodiments, the polymer comprises a cationic polymer, a polymer that is complexed with cations, or a co-polymer that includes a cationic polymer. In certain embodiments, the polymers comprise co-polymers of: (a) a cationic segment that electrostatically binds to negatively charged DNAO, and (b) a nonionic amphiphilic polymer.

In some embodiments, the cationic polymers include, but are not limited to pLys, pHis, pArg, N,N-dimethylaminoethyl methacrylate (DMAEMA), poly amido amine (PAMAM), polyethyleneimine (PEI), cationic cyclodextrin, cationic cellulise, cationic dextrin, cationic dextran, chitosan, gelatin, spermine, spermidine, cationic surfactants such as CTAB or combination thereof.

In some embodiments, the nonionic amphiphilic polymers include, but are not limited to, polyethylene glycol (PEG), polyoxazolines (POX), PVP (poly(N-vinylpyrrolidone), polyglycerols (PG), or combinations thereof.

In some embodiments, the polymers include co-polymers of a cationic polymer that electrostatically binds to negatively charged DNAO, and PEG. In certain embodiments, the polymers comprise PEG-pLys, PEG-pArg, or PEG-pHis co-polymers, or combinations thereof, and the PEG monomers may be any molecular weight. In various embodiments, the PEG monomers are at least 2 kDa, 3 kDA, 4 kDa, or 5 kDa in molecular weight. In other embodiments, the PEG monomers are between 2 kDa and 100 kDa in molecular weight, between 3 kDa and 100 kDa in molecular weight, between 4 kDa and 100 kDa in molecular weight, or between 5 kDa and 100 kDa in molecular weight. In some embodiments, the PEG comprises PEG monomers of at least 2 KDa or at least 5 kDa in molecular weight.

In some embodiments, the encasing of the DNAO in an inorganic shell includes combining the DOPM with an inorganic molecule precursor in an aqueous alcohol solution to form the inorganic shell encasing the DNAO.

In certain embodiments, the combining step includes combining the DOPM with an inorganic molecule precursor (e.g., tetramethyl orthosilicate (TMOS), tetraethyl orthosilicate (TEOS), or combinations thereof) and also a reducing agent in an aqueous alcohol solution to form the inorganic shell encasing the DNAO. In some embodiments, the inorganic shell includes silica, and in certain embodiments, is a silica shell. In some embodiments, the aqueous alcohol includes aqueous ethanol, and in some embodiments, the aqueous solution is at about pH 11.

In some embodiments, the inorganic shell includes gold, and in some embodiments, the inorganic shell is a gold shell. In some embodiments, the inorganic molecule precursor comprises chloroauric acid (H[AuCl₄]), gold (iii) acetate (Au(C₂H₃O₂)₃), or combinations thereof.

In some embodiments, the inorganic shell includes silver, and in some embodiments, the inorganic shell is a silver shell. In certain embodiments, the inorganic molecule precursor includes silver nitrate.

In some embodiments, the inorganic shell includes iron, and in some embodiments, the inorganic shell is an iron-based shell. In some embodiments, the inorganic molecule precursor includes ferric chloride (FeCl₂) and ferrous chloride (FeCl₃).

In some embodiments, the inorganic shell includes calcium phosphate, and in some embodiments, the inorganic shell is a calcium phosphate shell. In some embodiments, the inorganic molecule precursor includes calcium chloride and sodium phosphate dibasic dodecahydrate. In some embodiments related to calcium phosphate shells, the aqueous solution is at about pH 9.

In some embodiments, encasing the DNAO in an inorganic shell is carried out at a temperature of between about 4° C. and about 45° C., and in some embodiments, at about room temperature (or about 23° C.).

In certain embodiments of the methods, a reduction step may be included. In some embodiments, methods where the inorganic shell is a gold, silver, iron, platinum, or palladium shell, the methods may comprise inclusion of a reduction step. In these embodiments, the reducing agents reduce the higher oxidation state of metal (such as Au3+, Ag1+, etc.) in precursors (metal salts) to the ground state metal (Au0, Ag0). In these embodiments, a reducing agent may be added to the DOPMs in an aqueous solution. Due to the electrostatics, the reducing agent would surround the positively charged DOPMs, giving a high density of the reducing agent close to the DNAO surface. Upon the addition of the inorganic molecule precursor to the above mixture, the reduction of the metal ions would solely take place on the surface of the origami.

In some embodiments, the reducing agent includes sodium citrate, formaldehyde, sodium borohydride, or combinations thereof.

Sequencing

The introduction of massively parallel next-generation sequencing (NGS) has had an impact on biology and biomedical research fields due to its high sequence output and significantly reduced costs. NGS technologies typically involve generation of libraries (in which, e.g., genomic DNA is fragmented, and specific adaptor strands are attached for downstream processing), colony formation (in which many copies of each single library fragment are made on a bead or in a specific region of solid support), sequencing, and analysis. Colony formation typically uses polymerase chain reaction (PCR) to make polymerase colonies (polony), which involves locally amplifying a library fragment that is attached to a surface (e.g., bead, hydrogel matrix, or solid support) with primers bound to the surface. After colony generation, the complementary section of the library fragment is extended cycle-by-cycle using reversible terminators. The added nucleotides in each cycle are identified optically or electrically. The analysis step involves rebuilding the full nucleic acid sequence from the sequences of the plurality of fragments.

While significant improvement, both in terms of ease and yield, has been realized for nucleic acid library preparation, sequencing, and analysis, the same is not true for colony generation, which has remained challenging due, at least in part, to the inherent randomness associated with the binding of library fragments onto a solid support or bead.

The disclosure therefore also details constructs to address the challenges in colony generation, as well as methods for creating such constructs. Such constructs (or “particles”) are thermostable and enzyme-stable, mono- or polyfunctional inorganic nanoparticle structures (e.g., a structured nucleic acid polymer micelle with an inorganic shell), as described above, functionalized to isolate individual DNA fragments, followed by the enzymatic amplification of the fragment on that particle, in solution, without using surface immobilization to create a high-quality monoclonal polymerase colony (Polony) for use in DNA sequencing.

In one aspect, a construct is provided that, as described for the structured nucleic acid-based nanomaterials section above, includes (i) a structured nucleic acid polymer micelle (DOPM), which DOPM includes a structured nucleic acid (DNAO) template; one or more functional moiety attached to the DNAO template; and polymers that interact with nucleic acid present in the DNAO template to form the DOPM; and (ii) an inorganic shell surrounding the DOPM, in which the one or more functional moiety extends outside the DOPM and the inorganic shell. In some embodiments, the polymers that interact with nucleic acid present in the DNAO template to form the DOPM include (a) a cationic polymer that electrostatically binds to negatively charged DNAO; (b) a nonionic amphiphilic polymer; and (c) a functional terminal group; and wherein a DNA template for amplification is bound to the one or more functional moiety attached to the DNAO template; and wherein a primer pair is bound to the functional terminal group on the polymer, the primer pair including: (i) a first primer sequence complementary to either a forward strand or a reverse strand of the DNA template; and (ii) a second primer sequence complementary to a DNA strand opposite the first primer sequence; wherein one primer in the primer pair comprises a cleavable linker.

In various embodiments, the DNAO nucleic acid composition, polymer compositions (e.g., cationic polymers, nonionic amphiphilic polymers, co-polymers), inorganic shell types (e.g., silica, gold, silver, iron, calcium phosphate), functional moiety type and abundance, functional terminal groups, physical characteristics (e.g., molecular weights, segments of additional modularity, polymer layer thickness, inorganic shell thicknesses), reaction conditions, and the like, can include those as described in the section above.

In some embodiments, the construct includes an inorganic shell that includes silica, and in some embodiments, the construct includes a silica shell.

In some embodiments, the DNAO is stabilized by crosslinking, such as by bi-functional molecules that covalently react with DNA strands constituting the DNAO. In some embodiments, the DNAO is stabilized by crosslinking of DNA strands of the DNAO (such as by bi-functional molecules that covalently react with DNA strands constituting the DNAO), crosslinking of polymers that interact with nucleic acid present in the DNAO or crosslinking of both. For example, in embodiments that include PEG-PLys polymer, glutaraldehyde can be used to crosslink the lysine moieties on the PEG. In some embodiments, the construct is stable in non-aqueous, salt-free as well as organic solvents, and in some embodiments, the construct is stable in basic pH conditions and can sustain sudden pH changes, and in some embodiments, the construct is stable at 90° C. in salt-free buffer.

In another aspect, the disclosure provides a method of making the construct of the above aspect, including (a) providing a DNAO template having one or more functional moiety attached to the DNAO, the functional moiety including an oligonucleotide that is complimentary to single-stranded nucleic acid extensions placed at one or more locations on the DNAO, a polymer spacer, and a functional terminal group; (b) binding the DNAO with co-polymers including: (i) a cationic polymer that electrostatically binds to negatively charged DNAO, (ii) a nonionic amphiphilic polymer; and (iii) a functional terminal group bound to the nonionic amphiphilic polymer opposite to the bound cationic polymer that interact with nucleic acid present in the DNAO template to form the DOPM; (c) encasing the DNAO in an inorganic shell; (d) binding at least one DNA template to a functional terminal group bound to a functional moiety complimentary to single-stranded nucleic acid extensions placed at one or more locations on the DNAO; and (e) binding at least one primer pair to a functional terminal group bound to a functional moiety including a co-polymer.

In some embodiments, the method further includes step (f) binding the construct to a surface through a second functional terminal group on a second functional moiety that also comprises an oligonucleotide that is complimentary to single-stranded nucleic acid extensions placed at one or more locations on the DNAO. In some embodiments, the surface is in a flow cell, and in some embodiments, the flow cell is patterned or unpatterned.

In various embodiments of the method aspect above, the DNAO nucleic acid template composition, polymer compositions (e.g., cationic polymers, nonionic amphiphilic polymers, co-polymers), inorganic shell types (e.g., silica, gold, silver, iron, calcium phosphate), functional moiety type and abundance, functional terminal groups, physical characteristics (e.g., molecular weights, segments of additional modularity, polymer layer thickness, inorganic shell thicknesses), reaction conditions, and the like, can include those as described in the section above.

In some embodiments, the DNAO is stabilized by crosslinking, such as by bi-functional molecules that covalently react with DNA strands constituting the DNAO. In some embodiments, the DNAO is stabilized by crosslinking of DNA strands of the DNAO (such as by bi-functional molecules that covalently react with DNA strands constituting the DNAO), crosslinking of polymers that interact with nucleic acid present in the DNAO or crosslinking of both. For example, in embodiments that include PEG-PLys polymer, glutaraldehyde can be used to crosslink the lysine moieties on the PEG. In some embodiments, the construct is stable in non-aqueous, salt-free as well as organic solvents, and in some embodiments, the construct is stable in basic pH conditions and can sustain sudden pH changes, and in some embodiments, the construct is stable at 90° C. in salt-free buffer.

In certain embodiments of the methods, a reduction step may be included. In some embodiments, methods where the inorganic shell is a gold, silver, iron, platinum, or palladium shell, the methods may comprise inclusion of a reduction step. In these embodiments, the reducing agents reduce the higher oxidation state of metal (such as Au3+, Ag1+, etc.) in precursors (metal salts) to the ground state metal (Au0, Ag0). In these embodiments, a reducing agent may be added to the DOPMs in an aqueous solution. Due to the electrostatics, the reducing agent would surround the positively charged DOPMs, giving a high density of the reducing agent close to the DNAO surface. Upon the addition of the inorganic molecule precursor to the above mixture, the reduction of the metal ions would solely take place on the surface of the origami.

In another aspect, the disclosure provides a method for making a monoclonal cluster of a target oligonucleotides on a particle including (a) providing a construct according to the first aspect and related embodiments in this section labeled “Sequencing”; (b) binding a DNA template to a first functional terminal group on one or more functional moiety that binds to single-stranded nucleic acid extensions placed at one or more locations on the DNAO; (c) binding a primer pair to a functional terminal group on the one or more co-polymer functional moiety, wherein the primer pair comprises a first primer sequence and a second primer sequence, the first primer sequence being complementary to either a forward strand or a reverse strand of the DNA template, and the second primer sequence being complementary to a DNA strand opposite the first primer sequence; and one primer in the primer pair comprises a cleavable linker; (d) amplifying the DNA template on the surface of the construct by bridge amplification; and (e) cleaving the cleavable linker.

In some embodiments, the method is performed in solution, and in certain embodiments, the method further includes binding the construct to a surface through a second functional terminal group on one or more functional moiety that comprises an oligonucleotide that is complimentary to single-stranded nucleic acid extensions placed at one or more locations on the DNAO, where, in some embodiments, the surface is in a flow cell, and in some embodiments, the flow cell is patterned or unpatterned.

In some embodiments, library fragments captured on the hydrogel particle can be amplified after the hydrogel particle has been immobilized on a patterned surface. Operationally, this involves incubating the hydrogel particle on a single molecule array of capture sites created by DNA origami placement techniques after the library fragment has been covalently attached to the hydrogel particle. Subsequently, the amplification is carried out by incubating the substrate with polymer and dNTPs, followed by thermocycling for, e.g., 20-30 rounds.

In some embodiments, DNA origami-coated nanoparticles (e.g. spherical nanoparticles) can be used instead of creating an inorganic DNA nanoparticle using DNA origami as a template for inorganic material growth. Each spherical nanoparticle is designed to be coated with just a single, large DNA nanostructure. This enables a discrete number of ligands (as low as one) to be organized on the same nanoparticle. This approach enables single nanoparticles to interact with single molecules in a non-Poisson fashion. In embodiments, a magnetically active monofunctional nanoparticle can be made by using magnetic nanoparticles and coating them with a single, large DNA nanostructure.

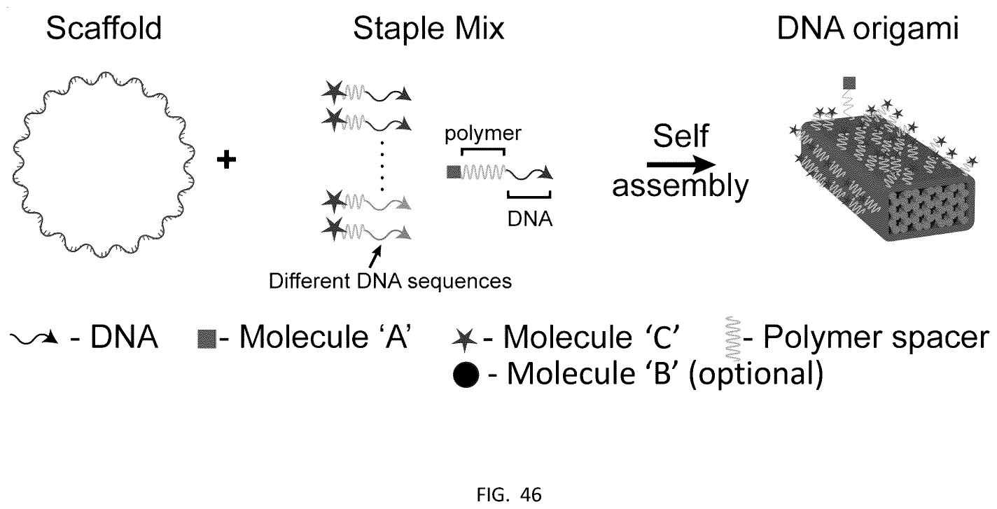

In some embodiments, rather than creating a hydrogel matrix on top of the DNA origami to introduce primer sequencing, the primers can be directly attached to the staples themselves as extensions (e.g., staple, polymer spacer, primers) or by having a particular reactive group on the staples themselves (FIG. 46). This approach, however, can limit the design space available as the sequence of the primer region needs to be very carefully designed to reduce the possibility of crosslinking within that region and minimize or eliminate aggregation through the ssDNA on the surface.

In some embodiments, local amplification of the captured library strand can be achieved isothermally using recombinase polymerase reaction.

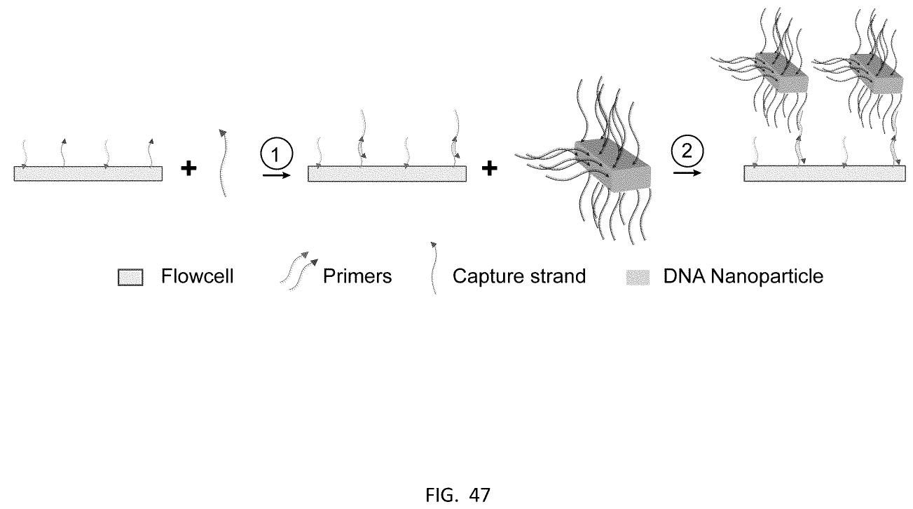

In some embodiments, rather than using substrates created by DNA origami placement techniques, existing flow cells (patterned or un-patterned, from companies such as Illumina, Ultima genomics, Singular Genomics, etc.) can be used to immobilize DNA nanoparticles carrying the monoclonal clusters (FIG. 47). A particle capture strand can be added to the flow cells, composed of one segment complementary to the flow cell's capture sites (e.g., P5 or P7) and another segment complementary to the primer binding region on the DNA nanoparticle. The excess, unbound strands are washed, and DNA nanoparticles are added. The DNA nanoparticles bind to the complementary regions of the capture strand, and the capture site on the flow cell and the primer binding region on the DNA nanoparticles are ligated chemically or enzymatically. The clusters are immobilized and ready to be sequenced and analyzed.

In another aspect, the disclosure also presents a method to create monoclonal clusters of nucleic acid fragments on DNA nanoparticles, in the solution phase, for DNA sequencing applications. In some embodiments, thermostable, enzyme-stable DNA origami particle include as many as two, three or more distinct functional modifications on its surface. In an embodiment the functional modifications can include, e.g.: (a) a single unit of a functional group that is designed to capture a single target DNA fragment from solution; (b) at least one pair of DNA primers to enable local amplification of the captured DNA fragment, and optionally, (c) a second functional group for immobilizing the DNA nanoparticle on a single-molecule array of capture sites on a substrate.

The DNA origami nanoparticle can capture a single DNA fragment from the solution and locally amplify it using the primer pairs, resulting in a particle covered with multiple copies of the captured DNA fragment. Finally, the nanoparticle can be immobilized onto, e.g., a single-molecule array of capture sites for sequencing via the second functional group, if present.

In some embodiments of the sequencing-related aspects, the construct comprises a plurality of copolymer functional moieties with functional terminal groups attached to a plurality of primer pairs, which, in certain embodiments, comprise the same sequences.

In some embodiments of the sequencing-related aspects, a functional terminal group (e.g., “A”) on the one or more functional moiety that can bind to single-stranded nucleic acid extensions placed at one or more locations on the DNAO can be utilized to bind to a template nucleic acid for amplification and sequencing. In some embodiments, DNA fragments are ligated with adaptor strands on both ends, which include a primer binding region complementary to the primer pairs described elsewhere. One of the adaptors has a functional group added to it (e.g., “A*”), which enables covalent bonding to the reactive group “A” on the functional terminal group.

In some embodiments of the sequencing-related aspects, a functional terminal group (e.g., “B”) on the one or more functional moiety that can bind to single-stranded nucleic acid extensions placed at one or more locations on the DNAO can be utilized to immobilize, capture, or otherwise bind the construct to a surface. In some embodiments, the functional terminal group can be bound to a patterned or unpatterned flow cell.

The DNA fragments are then incubated with the construct in a denaturing condition (such as elevated temperatures, pure water, or 7M urea) to minimize the interaction between the primer binding region on the fragments and the primers on the DNA nanoparticle while preserving the interaction between “A*” and “A”. After the DNA fragments have been tethered to the construct, excess untethered fragments are filtered out, and the solution is buffer exchanged into a non-denaturing buffer for thermocycling with a high fidelity polymerase and dNTPs.

Conjugates

The ability to detect and quantify low concentration biomolecules has clinical applications, allowing for both diagnosis and monitoring via the quantification of low concentration biomarkers within biological fluids. However, several key challenges remain in single-molecule quantification, particularly for bead-based methods. First, methods that utilize diffusion to immobilize targets upon a surface for readout are limited by diffusion at low concentrations. Diffusion limits the speed of target collection, thus requiring long incubation times. Furthermore, current bead-based quantification methods use beads that are functionalized with multiple capture units. These methods utilize a large excess of beads and rely on Poisson statistics to ensure that beads have captured at most one target. Lastly, there is no guarantee that every target molecule in a sample can be captured and, thus, detected.

The disclosure therefore also details a monofunctionalized DNA origami-nanoparticle (DNAO-NP) conjugate with signaling function (e.g., fluorescence), as well as a Poisson-independent method for high-sensitivity quantification of, e.g., biomolecules. To circumvent the reliance on Poisson statistics, the addressability of DNA origami can be used to ensure the DNA origami-nanoparticle conjugate is functionalized with only a single capture site. Such monofunctionality ensures that detection of binding of a capture molecule corresponds to the detection of a single target molecule.

In one aspect, the disclosure provides a monofunctional DNA origami nanoparticle conjugate construct, including: (a) an inorganic core; (b) one or more structured nucleic acid (DNAO) template attached to the core; (c) a binding functional moiety attached to the DNAO template for binding to a target analyte; and (d) one or more detection functional moiety attached to the DNAO template for detection of binding of a target analyte by the binding functional moiety.

The inorganic core can include, e.g., a magnetic core, a polymeric core (e.g., polystyrene), a metallic core (e.g., an oxide of iron, gold, silver), or a glass core, and in certain embodiments the inorganic core is magnetic. The inorganic core can be attached to the one or more structured nucleic acid (DNAO) templates, where one or more of the DNAO covers about 10%, or about 20%, or about 30%, or about 40%, or about 50%, or about 60%, or about 70%, or about 80%, or about 90%, or about 100% of the surface of the inorganic core. “Covering” the inorganic core can include steric hinderance of the surface of the core from the attached DNAO (such as attaching to the core with one or a small number of bonds, but occluding the surface beyond the binding site(s) due to steric hinderance), or, in some embodiments, attaching to the surface of the core with a plurality of bonds such that the one or more DNAO act as a shell to surround the inorganic core to a varying degree.

In some embodiments, the binding functional moiety is bound to a target analyte binding molecule, which can be any suitable moiety capable of binding to a target analyte, such as a binding protein, antibody, or aptamer. In some embodiments, the target analyte binding molecule includes an antibody.

A means of detecting a binding event is also provided and can include any suitable moiety capable of being detected, i.e., emitting or presenting a detectable signal (e.g., by light, chemical state, and the like). In some embodiments, the detection functional moiety includes one or more of a fluorescent moiety, a redox moiety, an enzymatic reporter and a nucleic acid tag, and in certain embodiments, the detection functional moiety includes a fluorescent moiety. In certain embodiments, biosensing and sequencing techniques, may be used to detect a binding event.

In another aspect, the disclosure provides a monofunctional DNA origami nanoparticle conjugate construct that includes (a) an inorganic core; (b) one or more structured nucleic acid (DNAO) template attached to the core; (c) a binding functional moiety attached to the DNAO template for binding to a target analyte; and (d) one or more detection functional moiety attached to the DNAO template for detection of binding of a target analyte by the binding functional moiety, wherein the DNAO further includes (i) polymers that interact with nucleic acid present in the DNAO template to form a structured nucleic acid polymer micelle (DOPM), and (ii) an inorganic shell surrounding the DOPM (DOPM-NP), in which the binding functional moiety and detection functional moiety extend outside the DOPM and the inorganic shell.

In some embodiments, the polymers that interact with nucleic acid present in the DNAO template to form the DOPM include (a) a cationic polymer that electrostatically binds to negatively charged DNAO; (b) a nonionic amphiphilic polymer; and, optionally, (c) a functional terminal group distinct from the binding functional moiety and the one or more detection functional moieties.

In various embodiments, the DNAO nucleic acid composition, polymer compositions (e.g., cationic polymers, nonionic amphiphilic polymers, co-polymers), inorganic shell types (e.g., silica, gold, silver, iron, calcium phosphate), functional moiety type and abundance, functional terminal groups, physical characteristics (e.g., molecular weights, segments of additional modularity, polymer layer thickness, inorganic shell thicknesses), reaction conditions, and the like, can include those as described in the section above.

In some embodiments, the construct includes an inorganic shell that includes silica, and in some embodiments, the construct includes a silica shell.

In some embodiments, the DNAO is stabilized by crosslinking, such as by bi-functional molecules that covalently react with DNA strands constituting the DNAO. In some embodiments, the DNAO is stabilized by crosslinking of DNA strands of the DNAO (such as by bi-functional molecules that covalently react with DNA strands constituting the DNAO), crosslinking of polymers that interact with nucleic acid present in the DNAO or crosslinking of both. For example, in embodiments that include PEG-PLys polymer, glutaraldehyde can be used to crosslink the lysine moieties on the PEG. In some embodiments, the construct is stable in non-aqueous, salt-free as well as organic solvents, and in some embodiments, the construct is stable in basic pH conditions and can sustain sudden pH changes, and in some embodiments, the construct is stable at 90° C. in salt-free buffer.

As with certain embodiment of the preceding aspect that includes a DNAO rather than a DOPM, the inorganic core can include, e.g., a magnetic core, a polymeric core, a metallic core (e.g., an oxide of iron, gold, silver), or a glass core, and in certain embodiments the inorganic core is magnetic. The inorganic core can be attached to the one or more structured nucleic acid (DNAO) templates, where one or more of the DNAO covers about 10%, or about 20%, or about 30%, or about 40%, or about 50%, or about 60%, or about 70%, or about 80%, or about 90%, or about 100% of the surface of the inorganic core, where “covering” again includes steric hinderance of the surface of the core or binding at a plurality of locations and acting as a shell to surround the inorganic core.

In some embodiments, the binding functional moiety is bound to a target analyte binding molecule, which can be any suitable moiety capable of binding to a target analyte, such as a binding protein, antibody, or aptamer. In some embodiments, the target analyte binding molecule includes an antibody.

A means of detecting a binding event is also provided and can include any suitable moiety capable of being detected, e.g., by light, chemical state, and the like. In some embodiments, the detection functional moiety includes one or more of a fluorescent moiety, a redox moiety, an enzymatic reporter and a nucleic acid tag, and in certain embodiments, the detection functional moiety includes a fluorescent moiety.

In another aspect, the disclosure provides a method for making a monofunctional DNA origami nanoparticle conjugate construct, including: (a) providing an inorganic core; (b) binding one or more DNA templates to the inorganic core; (c) providing a binding functional moiety for the DNA template to bind to a target analyte; and (d) providing one or more detection functional moieties for the DNA template for detection of binding of a target analyte by the binding functional moiety; (e) providing one or more staple strands; (f) providing buffer conditions amenable to self-assembly of the DNA template(s) into structured DNAO. In some embodiments, the DNA template is unstructured when bound to the inorganic core, and in some embodiments, the binding functional moiety and the one or more detection functional moieties are bound to the unstructured DNA template before addition of the staple strands. In certain embodiments, the binding functional moiety, and the one or more detection functional moieties comprise a staple strand sequence for incorporation into specific locations on the structured DNAO.

In some embodiments, steps (c) through (f) precede steps (a) and (b) such that a structured DNAO is formed prior to binding to the inorganic core. In certain embodiments in which steps (c) through (f) precede steps (a) and (b), the method further includes the steps of (i) binding the DNAO with polymers that interact with nucleic acid present in the DNAO template to form the DOPM; and (ii) encasing the DNAO in an inorganic shell (with bound polymers present, i.e., a DOPM), prior to steps (a) and (b), such that one or more inorganic shell-covered DOPM is formed prior to binding to the inorganic core.

As with the preceding aspects and embodiments related to monofunctional DNA origami nanoparticle conjugate constructs, in some embodiments, the one or more structured DNAO (or inorganic shell-covered DOPM bound to the inorganic core) covers about 10%, or about 20%, or about 30%, or about 40%, or about 50%, or about 60%, or about 70%, or about 80%, or about 90%, or about 100% of the surface of the inorganic core.

In some embodiments, the binding functional moiety is bound to a target analyte binding molecule, which can be any suitable moiety capable of binding to a target analyte, such as a binding protein, antibody, or aptamer. In some embodiments, the target analyte binding molecule includes an antibody.

A means of detecting a binding event is also provided and can include any suitable moiety capable of being detected, i.e., emitting or presenting a detectable signal (e.g., by light, chemical state, and the like). In some embodiments, the detection functional moiety includes one or more of a fluorescent moiety, a redox moiety, an enzymatic reporter and a nucleic acid tag, and in certain embodiments, the detection functional moiety includes a fluorescent moiety.

In assays using certain magnetic-based embodiments described above, a plurality of magnetic DNAO-NPs can be incubated with a sample containing the target of interest, resulting in a subset of the DNAO-NPs capturing single target biomolecules from solution. This solution is then incubated in a flow chamber with a substrate functionalized with detection groups, and a magnetic field is applied in order to rapidly immobilize the captured targets on the substrate. The magnetic field is then reversed, and the DNAO-NPs that have not captured targets are removed from the surface and washed away. Surface-based readout to generate a quantitative measure can be conducted via an appropriate detection method for the signaling groups present, e.g., fluorescence microscopy can be used to detect fluorophores, and voltammetry for a redox molecule. In some embodiments, the target is an antigen, the capture and detection groups are primary and secondary antibodies, and the signaling groups are fluorophores.

Compositions

In another aspect, the disclosure provides a composition including a plurality (e.g., 2, 3, 4, 5, 6, 10, 25, 50, 100, 1000, 5000, 10000, 100000, 1000000, or more) of constructs of any aspect or embodiment of the disclosure. In some embodiments, the plurality of constructs includes only inorganic shell-covered DOPM, only sequencing-based embodiments, or only DNAO-NP or DOPM-NP, while in some embodiments, combinations thereof.

In embodiments, the plurality of constructs is monodispersed, and may be provided in any suitable form, including in solution, frozen, freeze-dried, bound to a surface, and the like. In certain embodiments, the composition is in solution, and in some embodiments, the solution includes an organic solvent.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

### EXAMPLES

### Example 1: Feasibility of Silica Growth on DOPMs

As noted above, DNA origami have shown promise for fabrication of well-defined, nanometer-sized objects with selective addressability, i.e., control over placement of various materials within a DNA origami structure and on the surface of a DNA origami, with sub-nanometer precision. However, DNA structures can disassemble when removed from their assembly medium (typically an aqueous saline solution), and they have proved susceptible to high temperatures and extreme pH conditions. Prior attempts to remedy these shortcomings, imbuing stability over various environmental conditions, such as temperature, solution, pH, and solvents as possessed by inorganic nanoparticles, have not proved successful, in part due to the fundamental incompatibilities between conditions required for growth of inorganic materials (i.e., primarily organic solvents at high temperature or contaminate-free aqueous solutions) and conditions required by DNA origami (buffers/cations present and moderate temperatures).

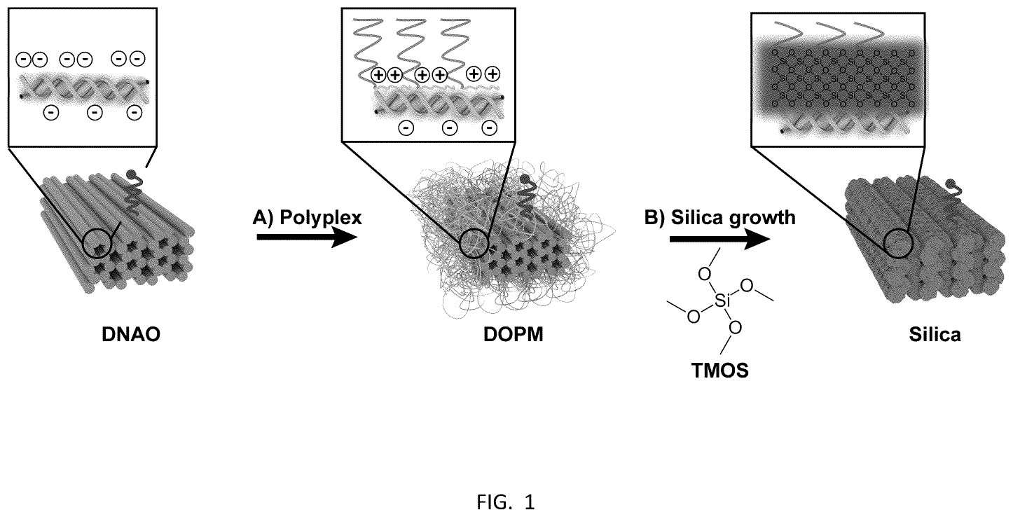

To demonstrate and, in later examples, optimize the synthesis of structured nucleic acid-based nanomaterials that exhibit significant thermostability and enzymatic stability, a well-known DNA origami in the shape of a brick (42-helix bundle or 42HB) were synthesized and auto-assembled with suitable staple strands as described in the Methods. The DNAO brick was converted into a polyplex micelle using poly(ethylene glycol)-b-poly(L-lysine) block copolymers (PEG-PLL, or PEG-pLys) with either 1, 5, or 20 kDa long PEG units, but with 10 lysine units in all cases (FIG. 1). The resulting polyplexes were characterized using dynamic light scattering (DLS). This bulk measurement technique enables observing changes in the hydrodynamic diameter (Z_{avg}, measured in nanometers) and the dispersity of particles for the entire duration of the growth as a function of time.

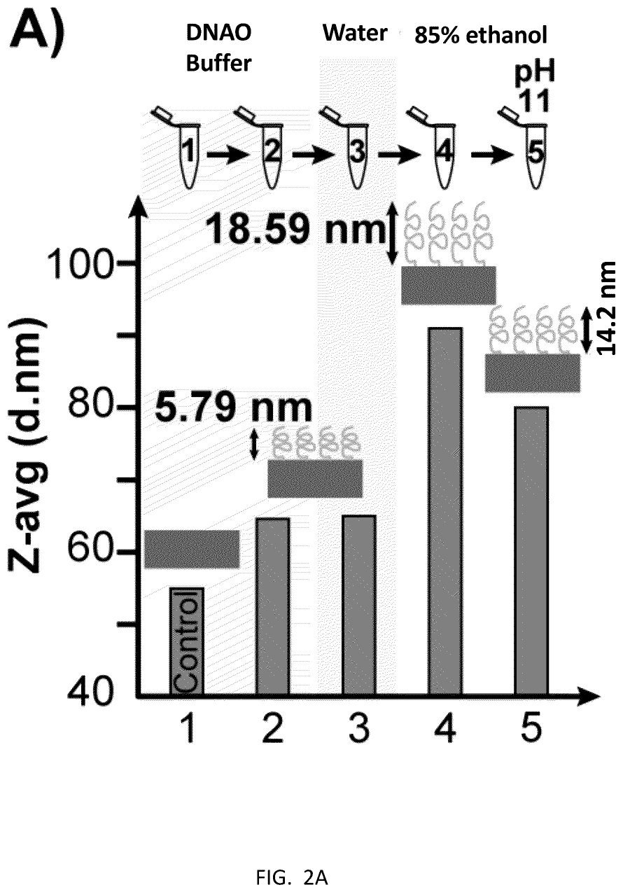

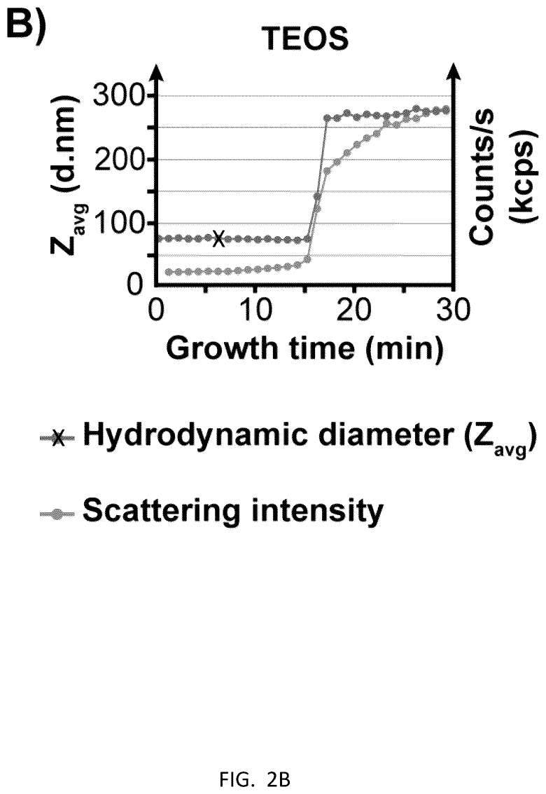

The DLS-based size analysis of 42HBs in the native buffers (10 mM Tris, 1 mM EDTA, and 5 mM MgCl2 maintained at pH 8.3) resulted in a single distribution peak, indicating a monodispersed solution with the Z_{avg} of 52.15±0.73 nm (FIG. 2A, tube 1). Here, despite the 42HBs having a cuboidal shape of dimensions 55×20×10 nm, the DLS algorithm simplifies it to an equivalent sphere of diameter Z_{avg} because of the Stokes-Einstein equation (see Methods, DLS).

The effect of different buffer conditions on the 42HB DNA origami polyplex micelles (DOPM) prepared using 5 kDa long PEG units (FIG. 2A, tubes 2-5) was assessed. From the DLS results indicated that in the native buffers, 42HB DOPMs were monodispersed with a Z_{avg} of 61.48±0.42 nm (FIG. 2A, tube 2), which is 17.89% (9.33±0.84 nm) more than the non-polyplexed 42HBs. This indicated that the thickness of the 5 kDa PEG shell around the 42HBs in the DOPMs is 4.67±0.42 nm.

Transferring the 42HB DOPMs into ultrapure water by buffer exchange led to a further increase in the size of the particles to a Z_{avg} of 63.73±0.51 nm (FIG. 2A, tube 3), while still exhibiting a single distribution peak in DLS, indicating that the particles are intact and stable in a salt-free solution. The additional 4.3% increase (2.25±0.66 nm) in hydrodynamic radius may be explained by the core of the DNA origami expanding due to the removal of the divalent cations known to condense and give structural rigidity to DNA origami. Finally, the 42HB DOPMs were transferred into an 85% ethanol solution. DLS analysis suggested that the particles were intact and non-aggregating, but the effective size of the particle increased to a Z_{avg} of 89.32±1.38 nm (FIG. 2A, tube 4). Not to be bound by theory, but the 25.59±1.47 nm increase in the thickness may be due to the PEG chains acquiring an extended conformation due to their interaction with ethanol rather than the compressed mushroom conformation observed in aqueous conditions. Concentrated ammonium hydroxide was added to the 85% ethanol solution with the 42HB DOPM to increase the effective pH of the solution to pH 11. A 9.8% decrease in the Z_{avg} to 80.55±1.03 nm (FIG. 2A, tube 5) was observed, while no change in the shape of the distribution was noted. Again, not to be bound by theory, but such behavior may indicate that the PEG chain length shortens in the presence of hydroxide ions, disrupting the interactions between the PEG chains and ethanol molecules. Hydroxide ions are highly reactive and can potentially react with both the PEG chains and the alcohol molecules, breaking the bonds between them and reducing the overall strength of the interaction.

Thus, these experiments demonstrated the structural viability and monodispersity of DOPMs in salt-free, partially non-polar, alcohol-water solutions, conditions that are amenable to inorganic deposition processes, such as the well-known Stöber process for silica growth.

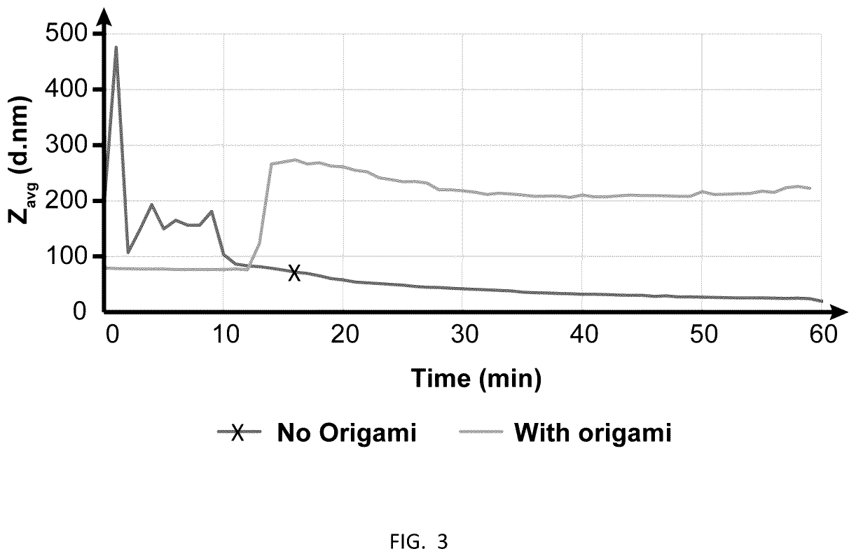

For silica growth, tetraethyl orthosilicate (TEOS) was added as a precursor to the 85% ethanol solution at pH 11 with the 42HB DOPMs, followed by incubation at room temperature for a fixed time interval. We characterized the growth using DLS (FIG. 2B). For the first 15 minutes of the reaction, no change in the hydrodynamic diameter of the particles in the solution was observed (Z_{avg} fluctuating between 77.29 nm and 81.46 nm, consistent with 42HB DOPMs), followed by a significant increase of Z_{avg} around 188 nm at the 16 min time point (FIG. 2B, upper curve). After this time, additional size distribution peaks were observed, indicating polydispersity and particle aggregation. The scattering intensity measured by the detector is directly proportional to the inorganic material growth that takes place on the surface of the DNAO. By analyzing the scattering intensity data as a function of time (FIG. 2B, bottom curve), a 34% increase in scattering intensity was observed during the first 15 minutes of incubation, which confirmed that prior to aggregation, the silica growth takes place in the solution, on the surface of the DNAO, indicating that small nucleation centers are constantly being generated in solution. At the same time, the silica shell grows on the DNA underneath the PEG layer on the 42HB DOPMs, which restricts any observable change to the Z_{avg}. The PEG shell also prevents the formation of aggregates until, after 15 minutes, the silica shell on the DNAO is thicker than the PEG shell. The silica growth was repeated without DOPMs templates as negative control while maintaining all the other solution conditions (FIG. 3). No condensed particles were observed during the 60 minute incubation, indicating that it is necessary to have a template in the solution that directs the condensation of the nuclei. From this, it was concluded that in the absence of any impurity (e.g., multivalent cations, buffering agents, and other non-participating molecules), the formation of side products is restricted, potentially limiting the availability of nuclei and the subsequent growth of the inorganic material to the DOPMs only.

These results indicated that templated growth of silica was possible on DOPMs in an alcohol-water solution. To have tighter control over the silica growth process, as well as to minimize spurious nucleation of silica nanoclusters, the following parameters were also investigated (as described in the following example): the pH of the reaction solution (pH 7, pH 8.3 or pH 11), the choice of solvent (methanol or ethanol), alcohol content (0%, 10%, 50%, 85% or 95%), the choice of the precursor (TMOS or TEOS), the concentration of the precursor with respect to the DOPMs, the length of PEG units in PEG-PLL (1 kDa, 5 kDa or 20 kDa), as well as pH transitions and the addition of PEG-silanes for reaction termination.

### Example 2: The Influence of pH

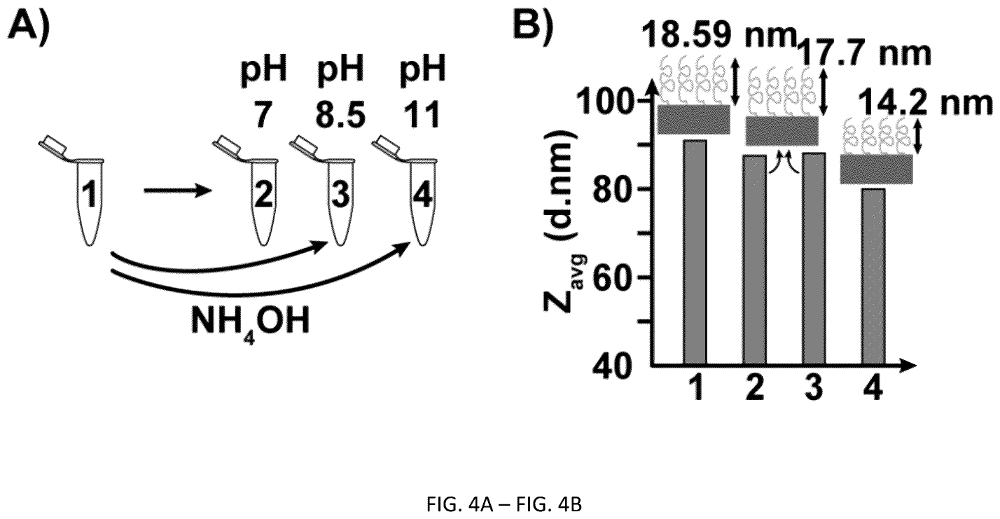

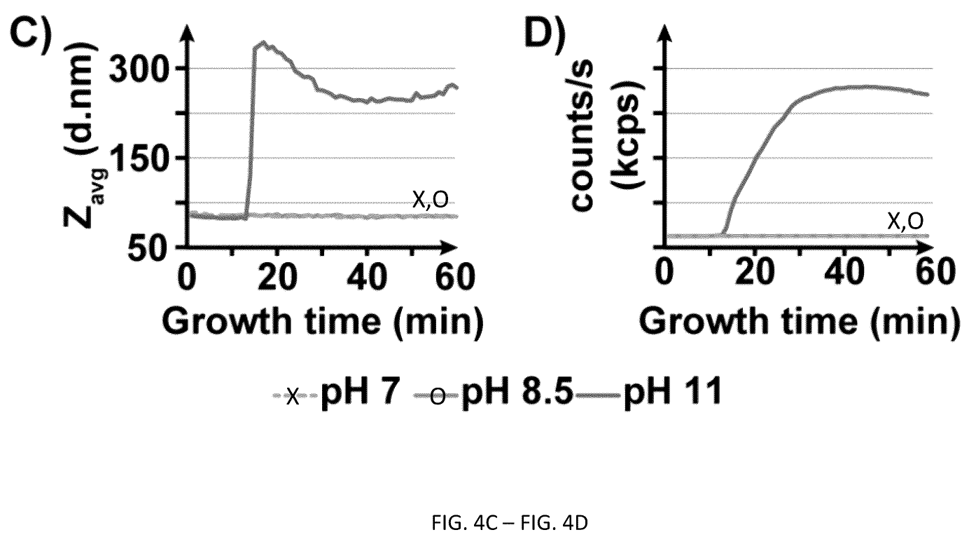

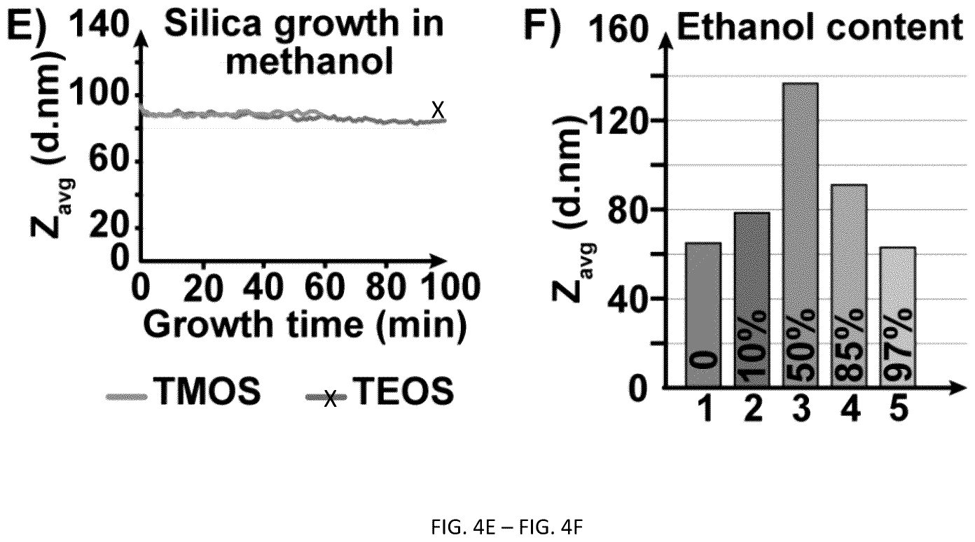

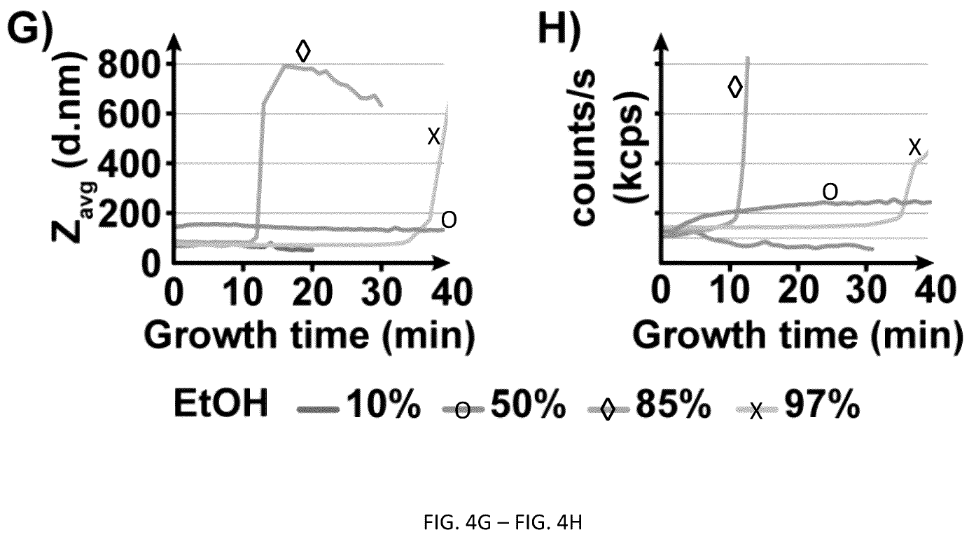

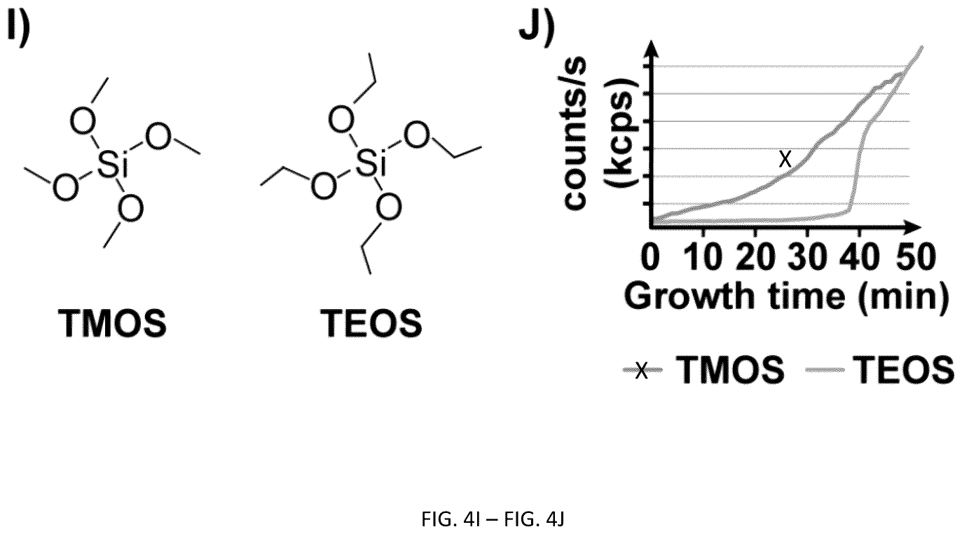

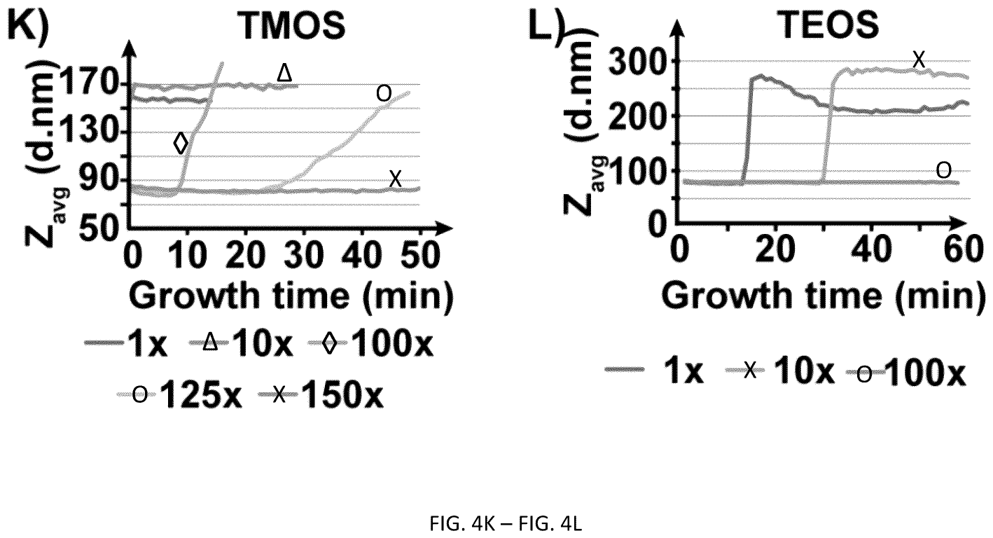

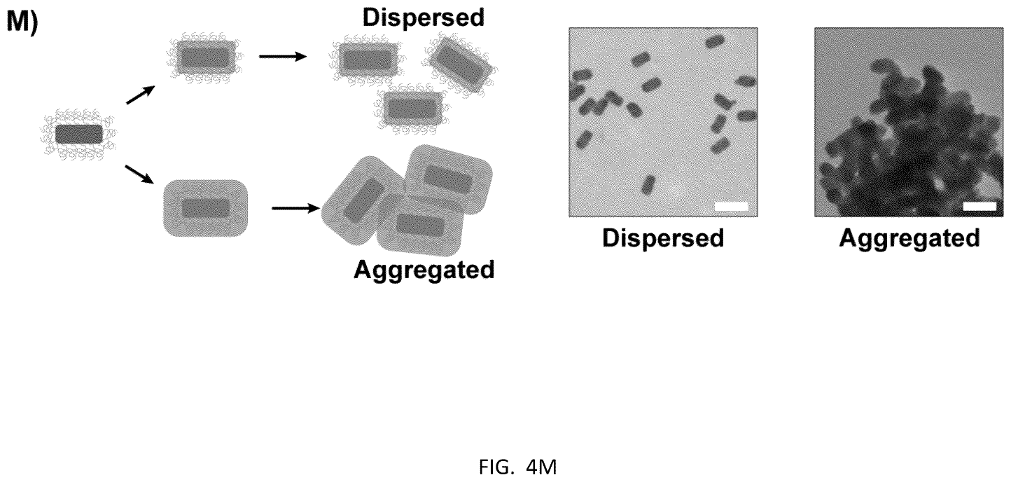

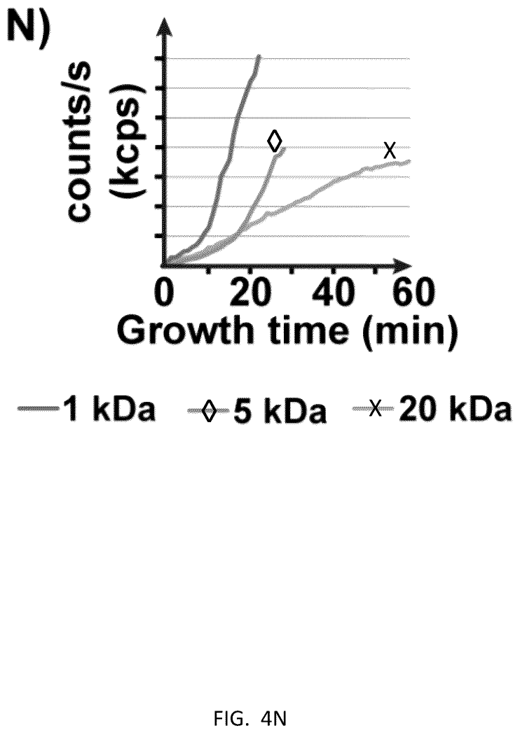

The effect of the different pH conditions (pH 7, pH 8.5, and pH 11) in 85% ethanol solution on 42HB DOPMs with 5 kDa PEG chain lengths (FIG. 4A) was then assessed. DLS measurements indicated that the Z_{avg} of the DOPMs at pH 7 and 8.5 were comparable at 87.56±1.2 nm and 88±2 nm, respectively (FIG. 4B). While for pH 11, the Z_{avg} decreased to 80±0.64 nm (FIG. 4B). Not to be bound by theory, but the decrease in Z_{avg} was attributed to the presence of excess OH− ion in the solution, which led to a change in PEG confirmation. Growth reaction at these pH conditions were then initiated by adding 1× concentration of TEOS (equivalent to 37.2 mM of TEOS per 1 nM of DOPMs). DLS results indicated that no silica growth had taken place at pH 7 or 8.5 over a period of 60 min (FIG. 4C, FIG. 4D, overlapping dashed curves). This is thought to be due to the slow rate of hydrolysis at these conditions that produce little to no nuclei. However, at pH 11, catalyzed by OH− ions, the hydrolysis of TEOS proceeds at a faster rate that reaches completion, immediately followed by condensation, which takes place at a steady rate due to a constant supply of monomers. These results indicated that pH 11 is suitable for silica shell growth.

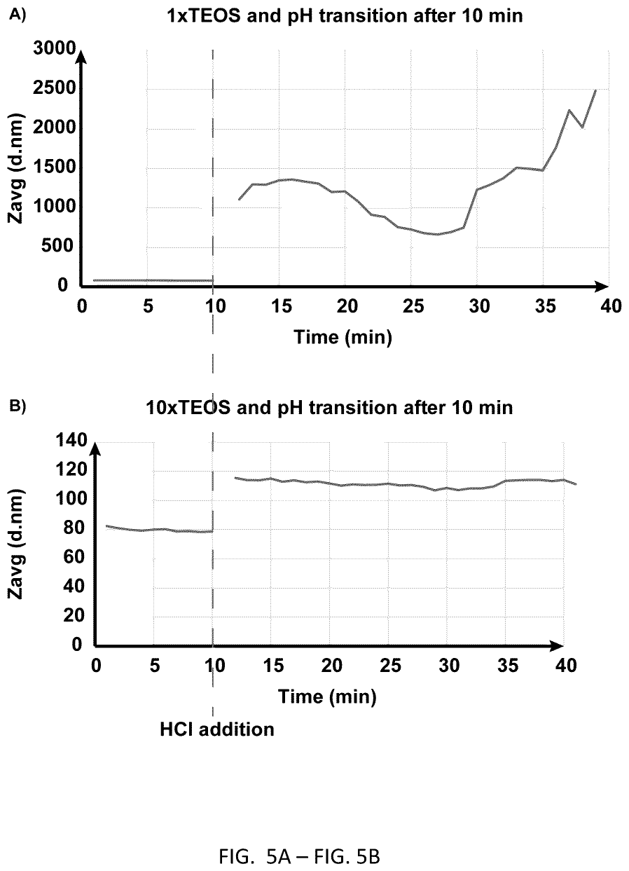

To further investigate the reliability of each step (hydrolysis, condensation, and growth) on the solvent pH condition, hydrochloric acid (HCl) was added after 10 minutes of reaction to change the pH of the solution from pH 11 to pH 7. This pH transition during the growth reaction led to an uncontrolled condensation that was observed as immediate aggregate formation (FIG. 5). In contrast to the reaction at pH 7, where no growth was observed (FIG. 4C, FIG. 4D), this uncontrolled condensation appears to be due to the pH transition, which appears to confirm that at pH 11, the reaction undergoes complete hydrolysis, and moving to pH 7 led to an increase in the rate of condensation resulting in the formation of gel-like products. It was concluded that such pH transitions could be used to terminate the reaction.

### Example 3: The Influence of Solvent

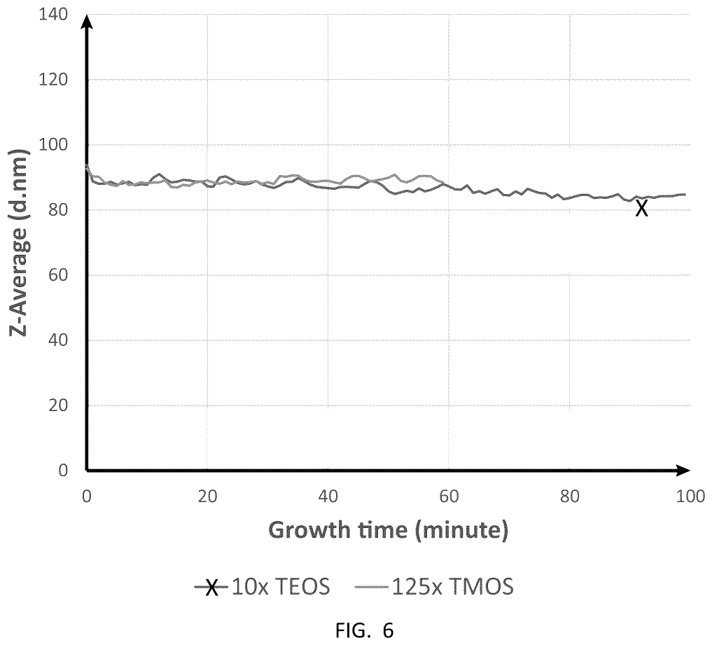

The dependence of the silica shell growth process on the solvent type was then investigated. Typically, the use of a precursor-miscible solvent facilitates the rate of hydrolysis. For these experiments, methanol and ethanol were compared as a solvent for silica growth, while keeping all the other parameters constant at 85% alcohol composition, pH 11, and using 42HB DOPMs with a 5 kDa PEG length. The growth reaction was initiated by adding tetramethyl orthosilicate (TMOS) or TEOS as silica precursors. DLS measurements of the samples with methanol as the solvent indicated no growth over 60 minutes and 100 minutes for either TMOS or TEOS as a precursor, respectively (FIG. 6). Methanol was not further tested as a solvent, and ethanol was utilized for all the subsequent studies.

### Example 4: The Influence of Alcohol/Water Content

The following ethanol compositions were assessed for their effect on silica growth: 10%, 50%, 85%, and 97%, using 42HB DOPMs with 5 kDa PEG lengths. Zₐvg of DOPMs was measured for these ethanol compositions, and an increase in the alcohol composition increased the Z_{avg} from 65±1.83 nm for pure water condition to 78.32±2.63 nm for 10% ethanol, with the maximum Z_{avg} of 136±5 nm at 50% ethanol, followed by a decrease to 90.97±2.7 nm and 62.88±0.9 nm for 85% and 95% ethanol compositions, respectively (FIG. 4E). Such observations may result from hydrogen bonding between the glycol units in the PEG chains with ethanol and water. The chains appear maximally stretched to 42 nm at 50%, depending on the ethanol composition. Following a decrease in the water content leads to the relaxation of the PEG chains to 19.41 nm and 5.36 nm at 85% and 97% ethanol composition, respectively. Interestingly, the PEG chains in both pure water and 97% ethanol composition exhibit comparable lengths of 3.52 nm and 5.36 nm, respectively (Figure FIGS. 4F, 1 and 5). We conclude that, independently, Both water and ethanol, independently, have a similar impact on the PEG chain lengths. For specific applications, a 50% ethanol composition appears to promote spatial separation of terminal functional groups on PEG chains farther away from the structure, making them more accessible for target molecules.

Growth reactions were then initiated at these ethanol compositions and at pH 11 by adding 1× concentration of TEOS. Z_{avg} and the scattering intensity over a 40 minute reaction indicated that no growth took place at 10% and 50% ethanol content (FIG. 4G, FIG. 4H; no changes were observed in either even after 180 min of incubation, data not shown). Growth was observed to be nearly 3 times slower at 97% ethanol compared to 85%, as the aggregation time point for 97% ethanol appears around the 35 minutes timepoint compared to 12 minutes for the 85% composition. This may due to a slower rate of hydrolysis at a low water content setting (3%), which leads to the lack of the monomers required for growth. The 85% ethanol composition appeared well suited for silica growth.

### Example 5: The Influence of TMOS Precursor Versus TEOS Precursor

The influence of precursor on silica deposition was tested by comparing TMOS to TEOS (FIG. 4I) on 42HB DOPMs with 5 kDa PEG lengths, keeping all other parameters constant at 85% ethanol and pH 11. Growth reactions were performed for 50 minutes and results plotted as scattering intensity as a function of time (FIG. 4J). DLS results indicated that the reaction proceeds about 4-5 times faster for TMOS than TEOS. The plot suggested that the silica growth using TMOS followed a roughly linear path (FIG. 2 4J, upper curve), while for TEOS, the growth was switch-like, with minimal growth until an inflection point at about 38 minutes where large aggregates start to form (FIG. 4J, bottom curve). This behavior may be due to the slower rate of hydrolysis for TEOS compared to TMOS, which delays the condensation of silica. The results indicated that while either precursor is suitable for silica growth, TMOS appears to provide a better opportunity for facile and controllable growth.

To further assess the two precursors, the effect of their concentrations on silica growth was determined by measuring the aggregation time points for varying concentrations of TMOS or TEOS, while keeping all the other parameters constant at 85% ethanol, pH 11, and 5 kDa PEG. The precursor concentration was varied between 1× and 150×, where x represents the dilution factor, with 1× representing the highest concentration corresponding to 9,300 molecules per nm² of the origami or 37.2 mM of precursor per 1 nM of origami. Silica growth was conducted for 50 minutes and the Z_{avg} was plotted as a function of time. 1× and 10× concentrations of TMOS (FIG. 4K) appeared to lead to aggregation immediately after precursor addition. In contrast, 1× and 10× concentrations of TEOS exhibit slower growth, and the structures aggregated after 16 and 31 minutes, respectively (FIG. 4L).

### Example 6: The Influence of Polymer Chain Lengths

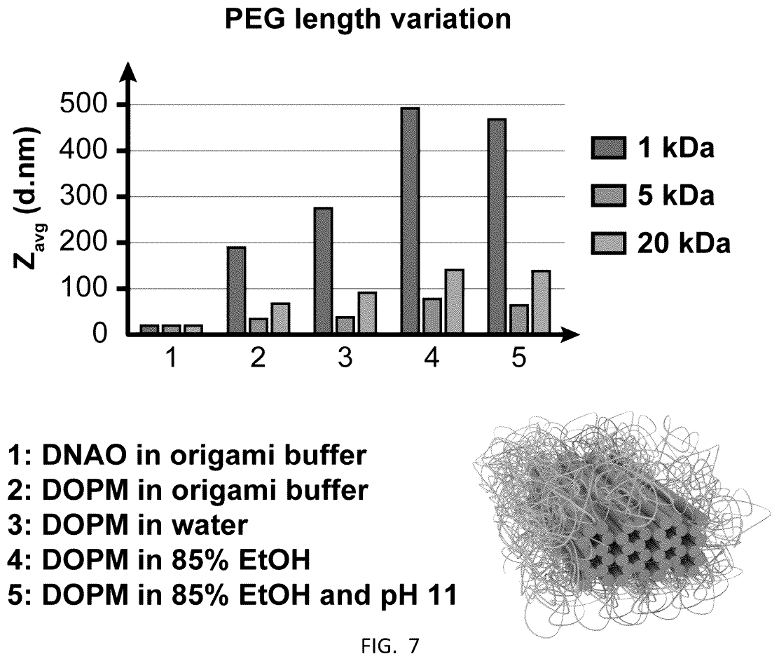

The impact of the PEG chain on the growth process was then investigated. Three variations of PEG-PLL were used with a constant cationic segment formed with 10 lysine repeats while changing the PEG chain lengths to 1 kDa, 5 kDa, or 20 kDa, under the hypothesis that the external PEG shell may prevent aggregation during the silica growth process, keeping the structures monodispersed and allowing a controlled deposition of silica (FIG. 4M). The Z_{avg} of 42HB DOPMs with varying PEG chain lengths in different buffer conditions were measured (FIG. 7). In the native buffers, the Z_{avg} for 42HB DOPMs with 20 kDa PEG increased to 83±1.4 nm from 52.15±0.73 nm for 42HB DNAO and 61.48±0.42 nm for 42HB DOPMs with 5 kDa PEG. Like the 5 kDa PEG described elsewhere, the Z_{avg} in 85% ethanol for DOPMs with 20 kDa PEG increased by 46±0.9 nm to 128.8±2.5 nm. This increase was proportional to the increase in the length of the PEG chain, with an average chain length of 20 kDa in 85% ethanol being 38.33±1.3 nm, and 18.59±0.78 nm for 5 kDa. However, the transition to pH 11 solution decreased Z_{avg} slightly to 127.5±1.5 nm. During this study, aggregation was observed for 1 kDa PEG, even in native buffers (FIG. 7). Not to be bound by theory, smaller PEG lengths (1 kDa) may be insufficient to avoid aggregation, and the structures form aggregates during the polyplex micellization process. Thus, longer PEG lengths appear to increase the stability and monodispersity of the structures.

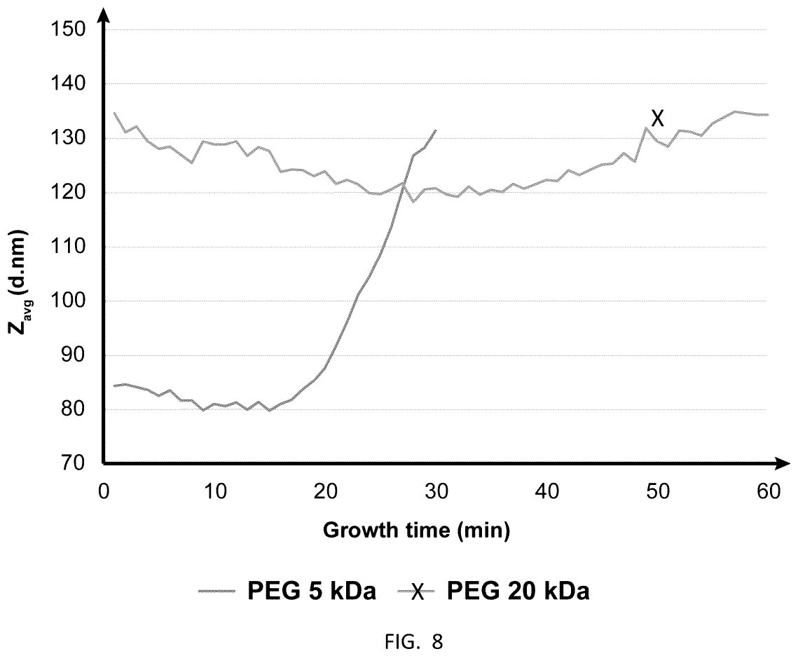

The effects of PEG chain length on silica growth was further investigated on 42HB DOPMs by utilizing PEG chain lengths of 1 kD, 5 kD and 20 kD, while keeping all other parameters constant at 85% ethanol, pH 11, and 125× concentration of TMOS as a precursor. Silica shell growth was observed for 60 minutes. The scattering intensity data were plotted as a function of time (FIG. 4N, corresponding Z_{avg} data in FIG. 8). For 1 kDa PEG the growth occurred steadily during the initial 10 minutes of incubation, followed by rapid growth causing aggregation in addition to the aggregates formed in the polyplex micellization step (FIG. 4N, left hand curve). Notably, for the 1 kDa PEG length, even prior to the silica growth, the 42HB DOPMs exhibited polydispersity and a cumulative Z_{avg} of 336.5±11 nm. From these results, it was determined that the 1 kDa PEG lengths were not well suited for the constructs described in the disclosure. In the case of 5 kDa PEG, steady growth was observed until the aggregation timepoint of about 16 min. 20 kDa PEG exhibited steady growth for the entire incubation, which suggests that longer PEG lengths maintain a monodispersed solution enabling uniform shell growth (FIG. 4N, middle curve for 5 kDa and right hand curve for 20 kDa; corresponding Z_{avg} data in FIG. 8). To maintain a similar steady growth condition for 5 kDa PEG, a lower precursor concentration may be helpful. However, a lower concentration of precursor results in thinner silica shells as the rate of silica shell growth is directly proportional to the concentration of the precursor.

### Example 7: The Influence of Precursor Concentration

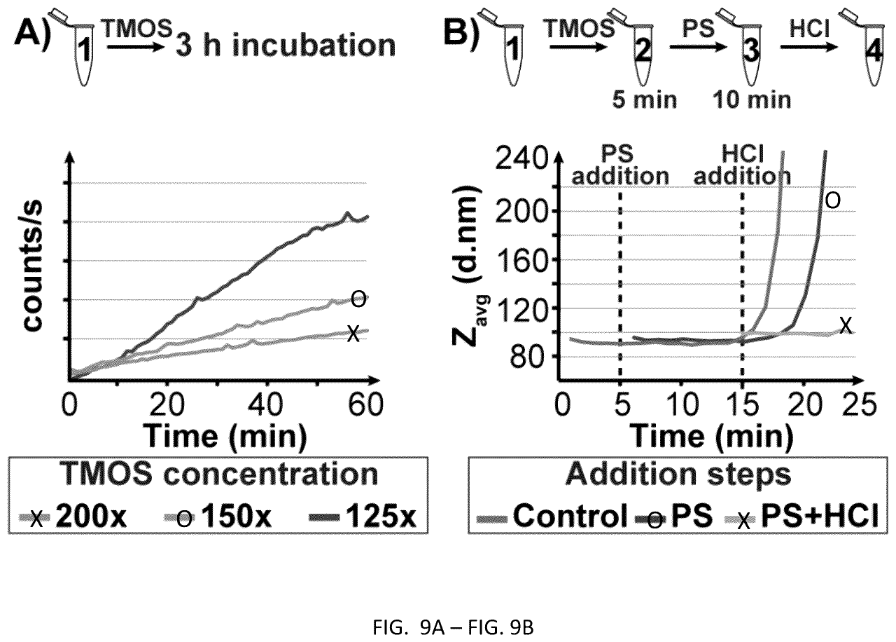

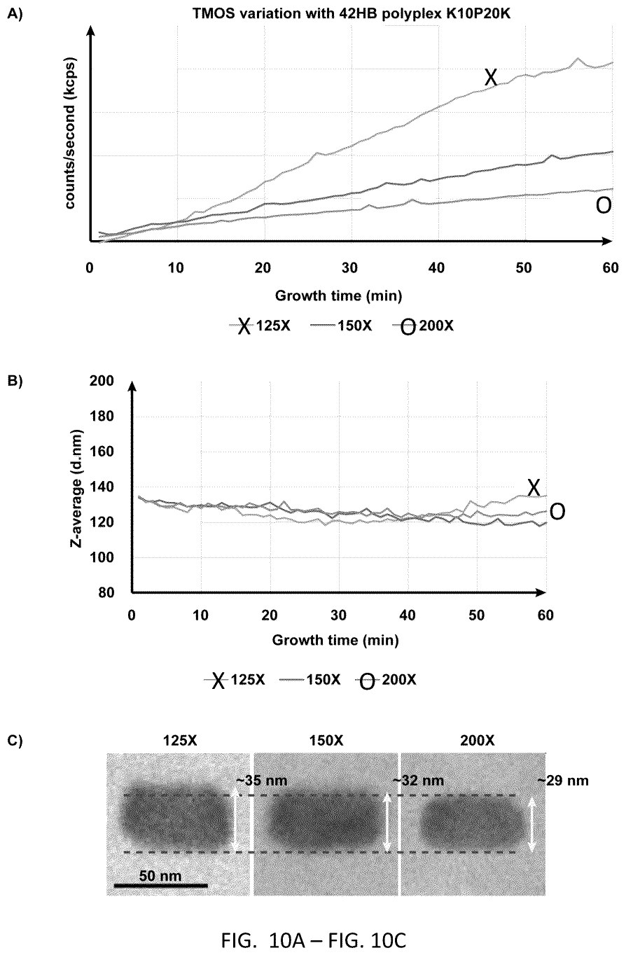

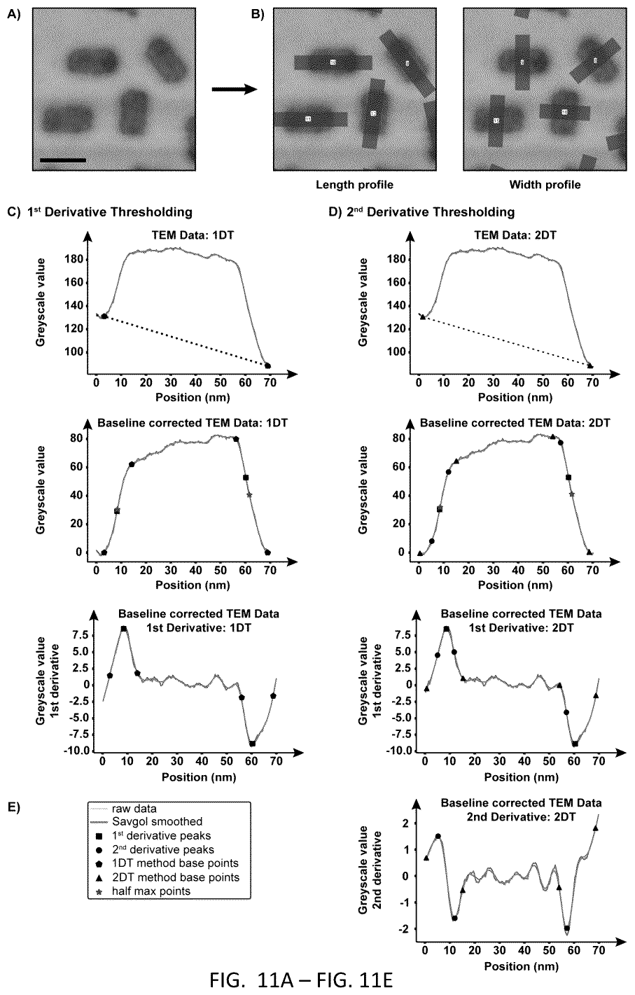

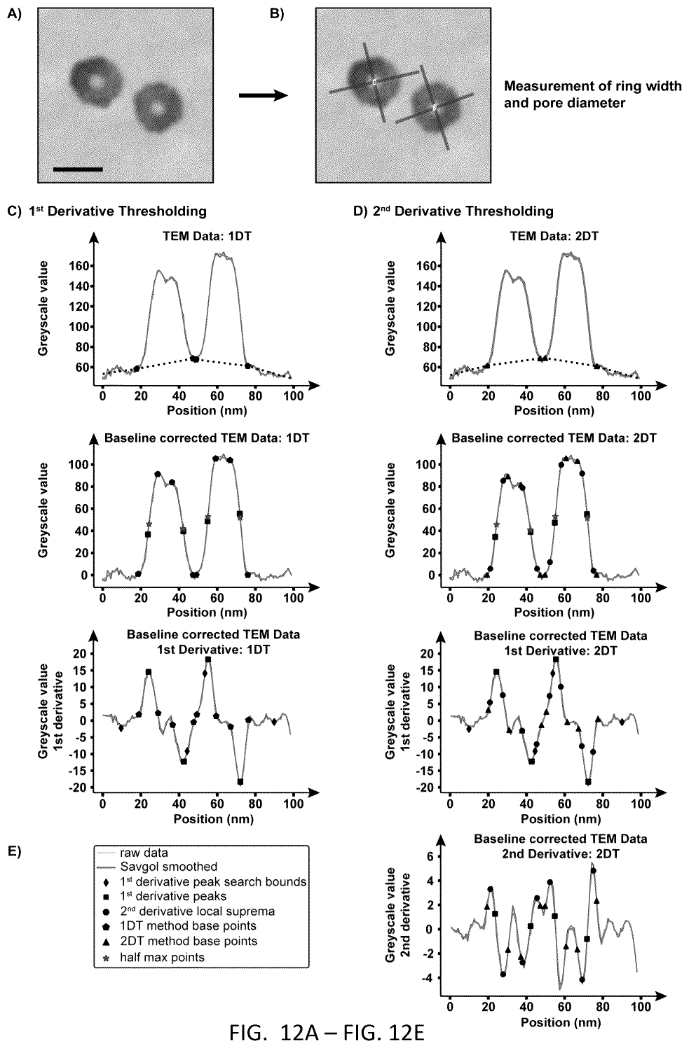

The effects of precursor concentration was investigated by measuring silica shell growth at varying TMOS concentrations for 20 kDa PEG lengths while keeping all other parameters constant (FIG. 9A and FIG. 10). Longer PEG chain lengths appeared to ensure a steady rate of silica deposition despite the increased rate of silica condensation due to higher precursor concentrations. By maintaining a constant growth time of 3 hours, different shell thicknesses were obtained at different TMOS concentrations. Specifically, 6 nm growth was observed for 125×TMOS, 5.2 nm for 150×TMOS, and 3.63 nm for 200×TMOS concentrations. (FIG. 9C; FIG. 11 and FIG. 12 for additional details regarding size measurements). These results indicated that the silica shell thickness could be tuned by performing silica shell growth at lower precursor concentrations, followed by longer incubation times, and undergoing complete exhaustion of the precursor to stop the reaction. However, reliance on the exhaustion of precursors for reaction termination increased reaction times (3-24 hours) and was accompanied by side products (e.g., non-templated silica particles, aggregates), which reduced reproducibility as well as introduced the need for downstream purification.

### Example 8: The Influence of Capping Agents and pH Transitions

Alternative approaches to controlling the thickness of silica layer growth include pH transitions using acids like HCl to terminate the reaction, or the use of capping agents like PEG-silanes (“PS”; 2-[methoxy (polyethyleneoxy) propyl] trimethoxy silane with 6-9 (CH₂CH₂O) units). To investigate these alternatives, the impact of the addition of PS, HCl, or a combination of both on the growth reaction were investigated.

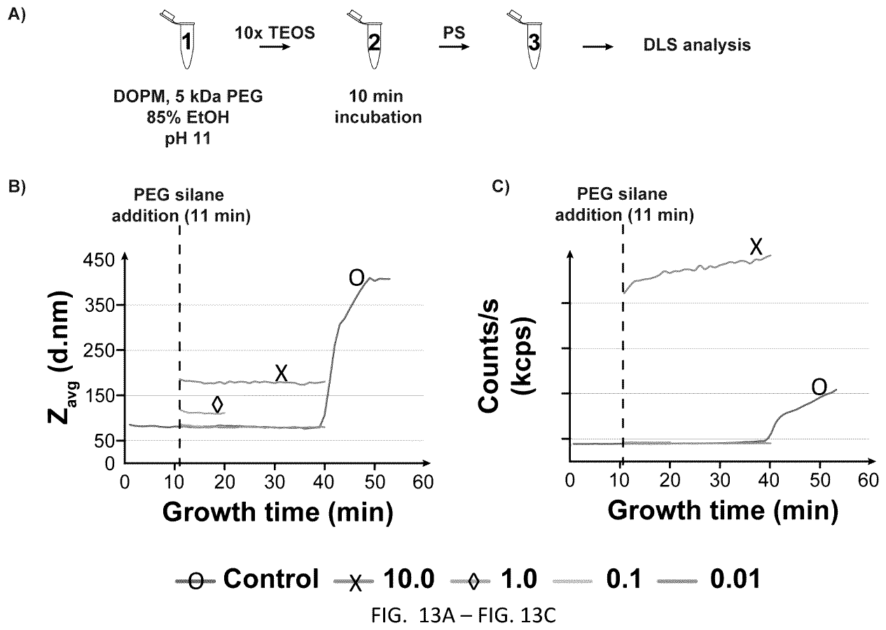

Introduction of the capping agent and pH change were investigated independently. Silica shell growth was conducted on 42HB DOPMs with 5 kDa PEG lengths, using a 10× concentration of TEOS as the precursor. At first, silica shell growth was terminated by adding varying concentrations of PS after performing the growth for 10 min (FIG. 13A). The PS concentration was relative to the precursor concentration, described by the PS/precursor molar ratio. For stoichiometry optimization, the ratio was varied between 10:1 and 0.01:1. From the observed Z_{avg} and scattering intensity plots it was concluded that the addition of PS stops the growth reaction for all the variants (FIG. 13B and FIG. 13C). Further, the Z_{avg} remained constant over the following 24 hours (and 75 hours for the variant with 1:1 PS concentration), confirming that PS terminates the growth reaction.

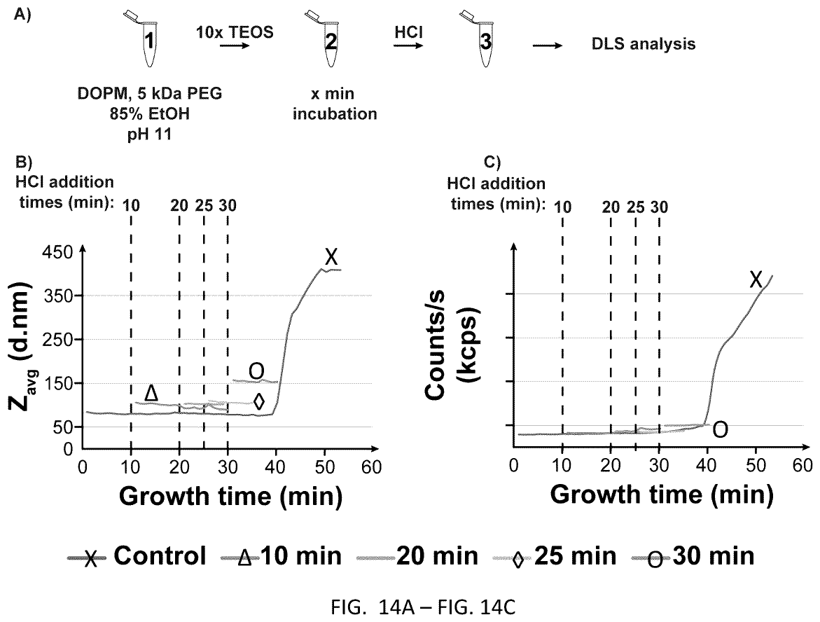

Termination of silica shell growth by adding HCl (in place of PS) to the above reaction using TEOS was investigated. Adding HCl changed the solution pH from 11 to 7 (FIG. 14A). Not to be limited by theory, but the transition to pH 7 was thought to minimize the hydrolysis rate, which would reduce the monomer supply, and, in contrast, the higher rate of condensation would lead to rapid growth followed by reaction termination. Growth for varied times (10, 20, 25, and 30 min) was carried out, followed by the addition of HCl. From the Z_{avg} and scattering plots, the pH transition appeared to lead to the termination of the growth reaction (FIG. 14B and FIG. 14C). The Zₐvg of the resulting structures was directly dependent on the time points at which HCl was added.

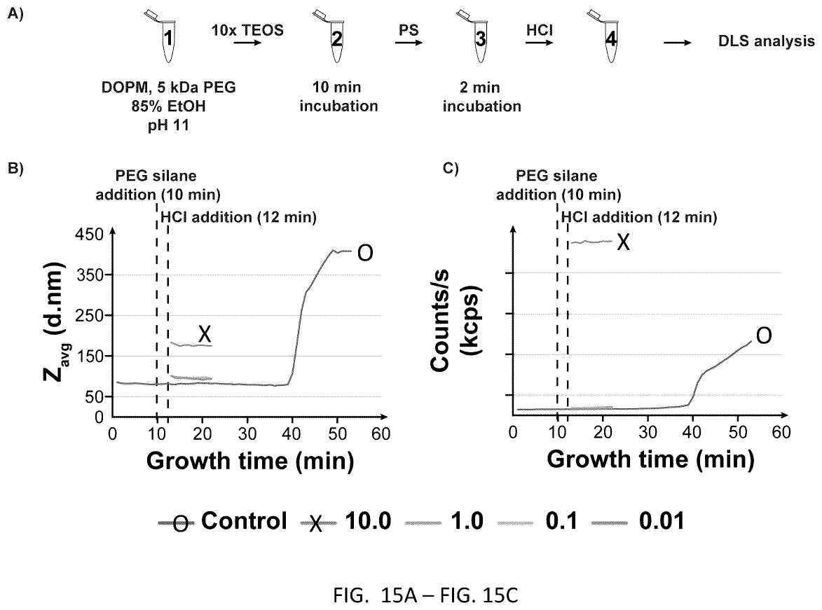

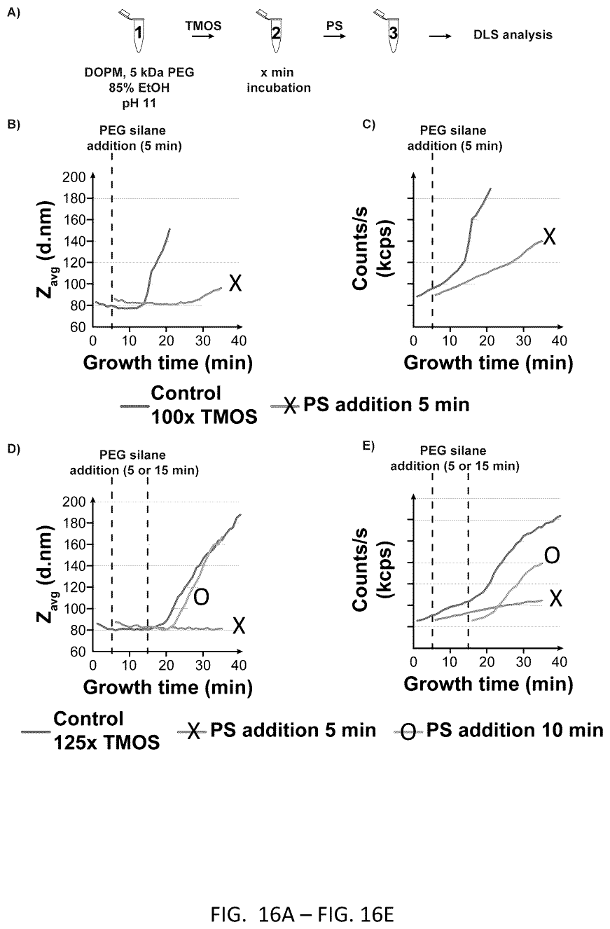

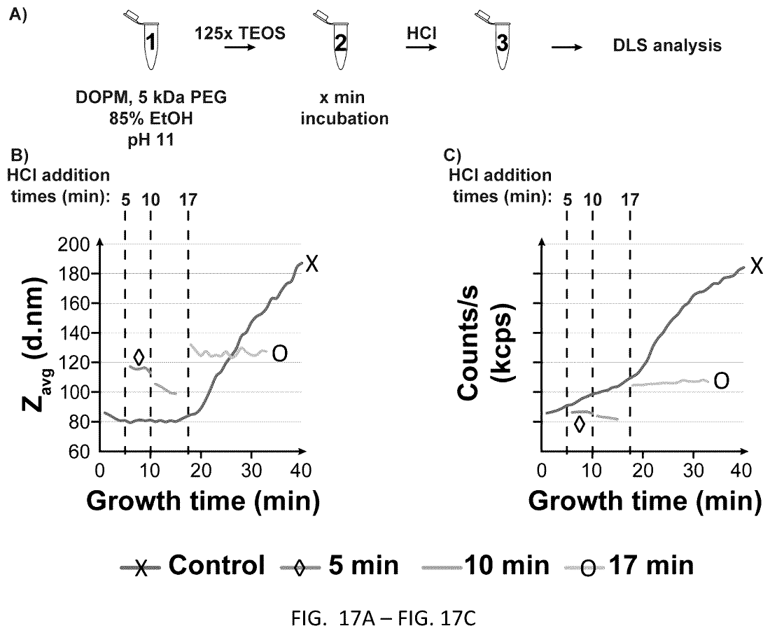

Finally, optimal PS concentration (0.1:1) to stop the growth reaction using TMOS (in place of TEOS) as a silica precursor was investigated. In contrast to the above observations, adding PS did not terminate the silica shell growth. Instead of stopping the silica growth completely, the capping agent only delayed the aggregation by 4 min (FIG. 9B, right hand curve). The inability of PS to terminate the growth using TMOS as a precursor may be due to the higher rate of hydrolysis of TMOS compared to TEOS, which out-competes the addition of a capping agent to the growing shell. Similar observations were made for reaction termination using HCl, where, despite termination, the shell growth was observed to be 5 times faster compared to TEOS (FIG. 14 to FIG. 17). To circumvent this, a combination of adding PS and HCl was utilized. PS was added after 5 minutes of growth reaction, and HCl was added after 10 minutes (FIG. 9B). The DLS results confirmed the termination of the growth reaction (FIG. 9B, bottom, flat-line curve), and a shell thickness of 4.23 nm was obtained with a total reaction time of 15 min (FIG. 9C).

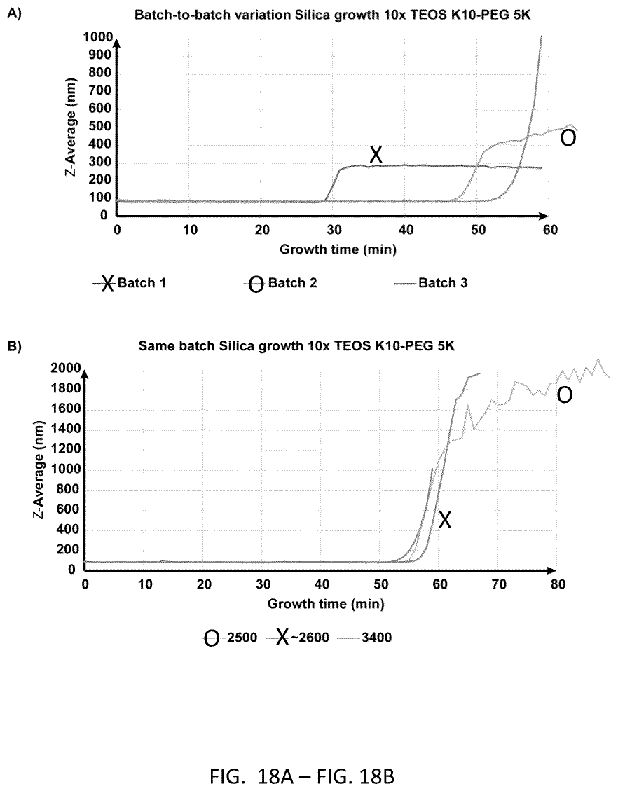

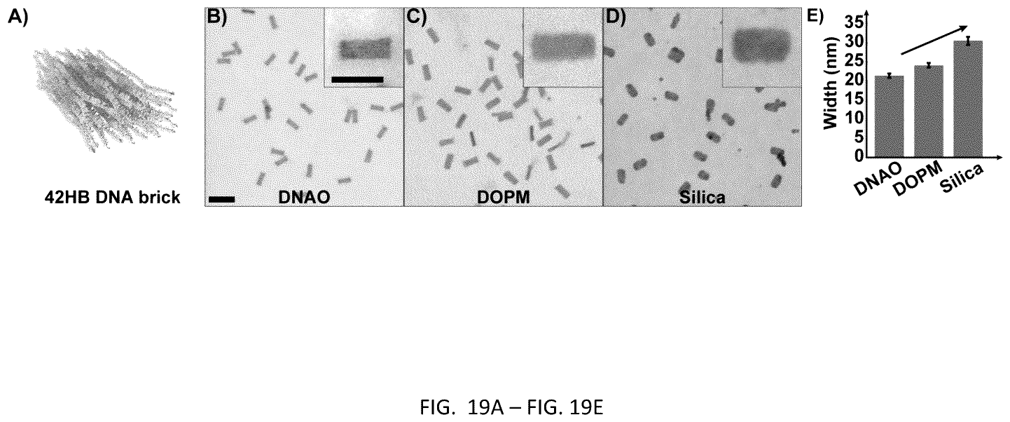

It is notable that the time points for adding PEG-silane and HCl may be optimized using DLS data, as time points can vary from batch to batch (FIG. 18) and across different structures. However, such optimizations are typically not necessary for growth reactions performed using lower precursor concentrations that do not require the addition of PS and/or HCl.

### Example 9: Optimized Growth Conditions and Characterization

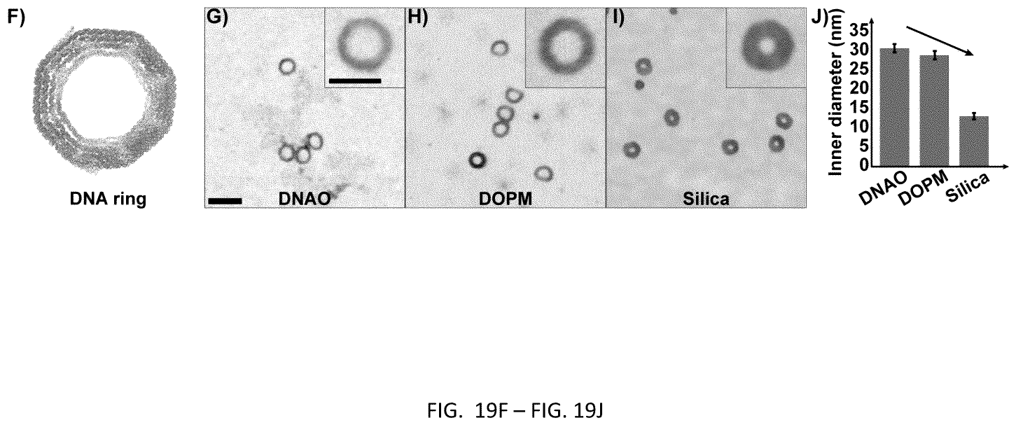

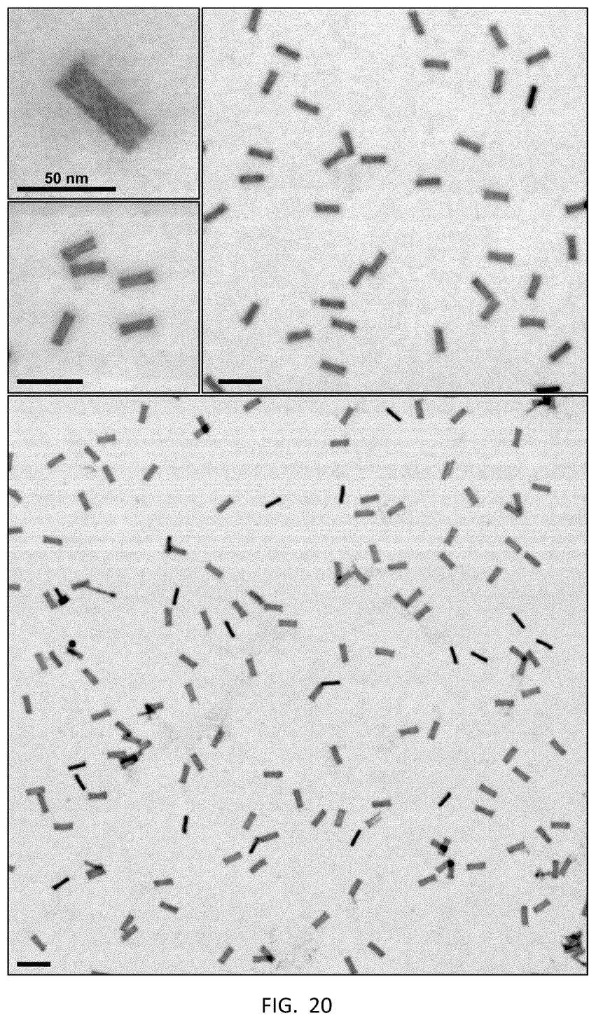

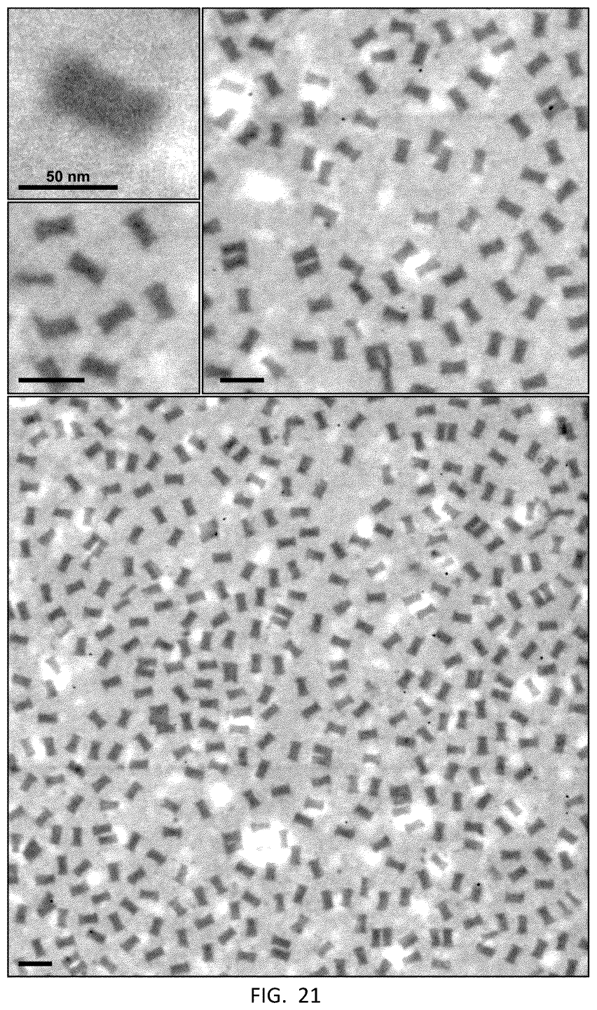

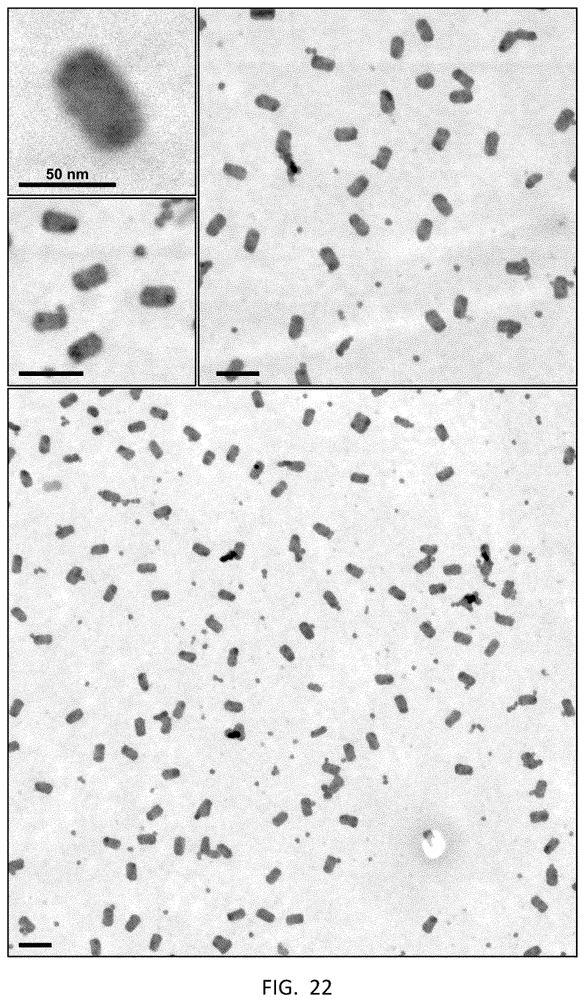

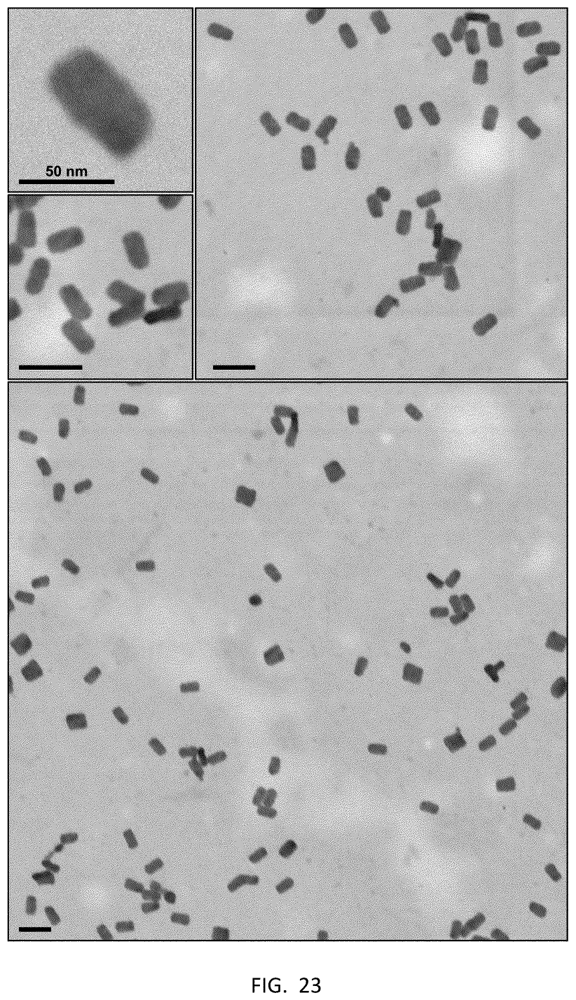

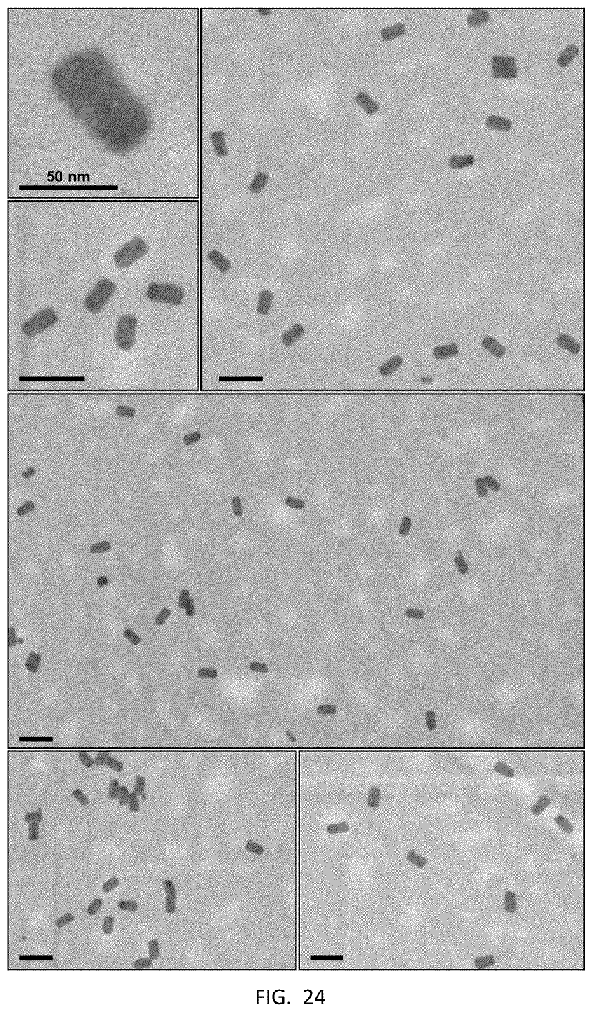

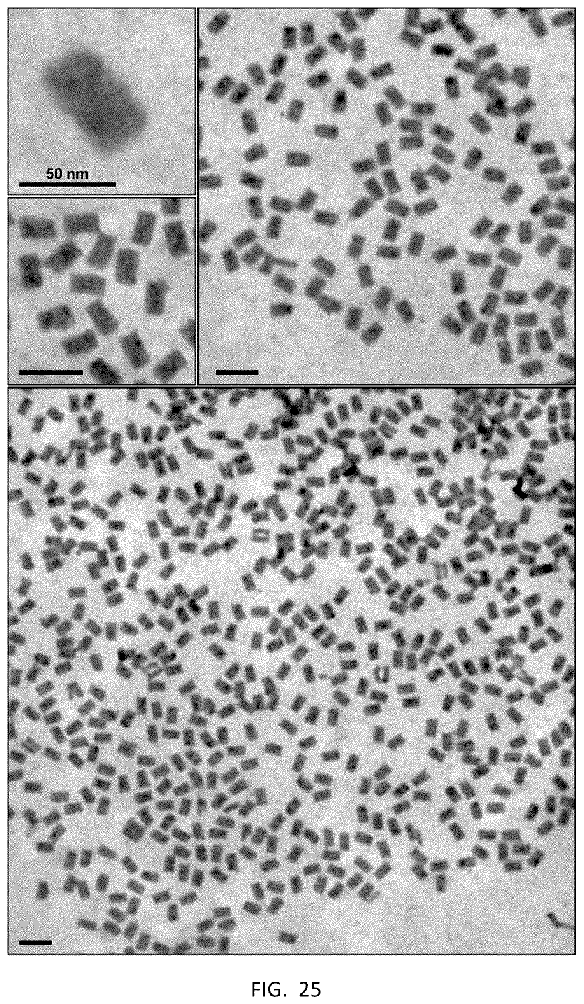

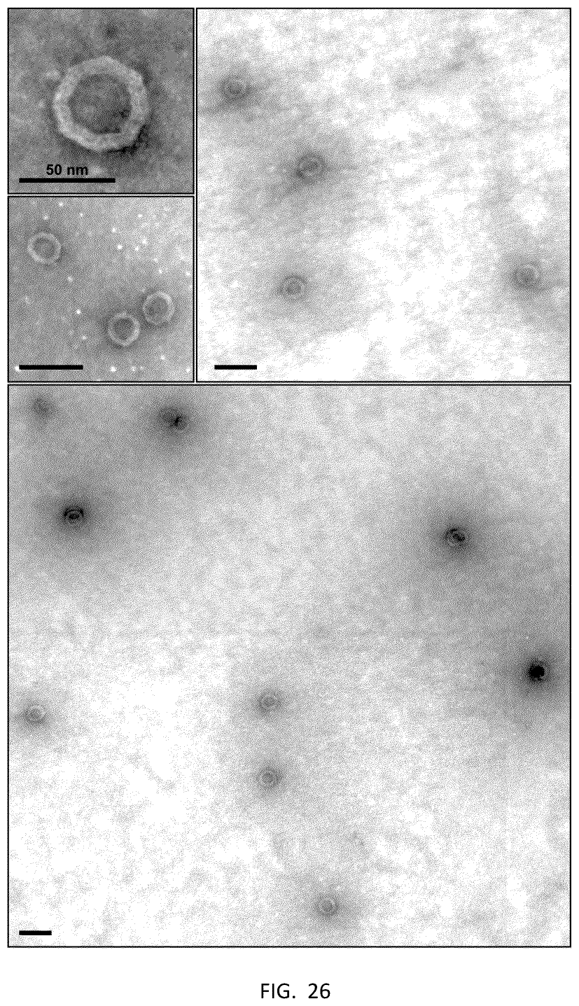

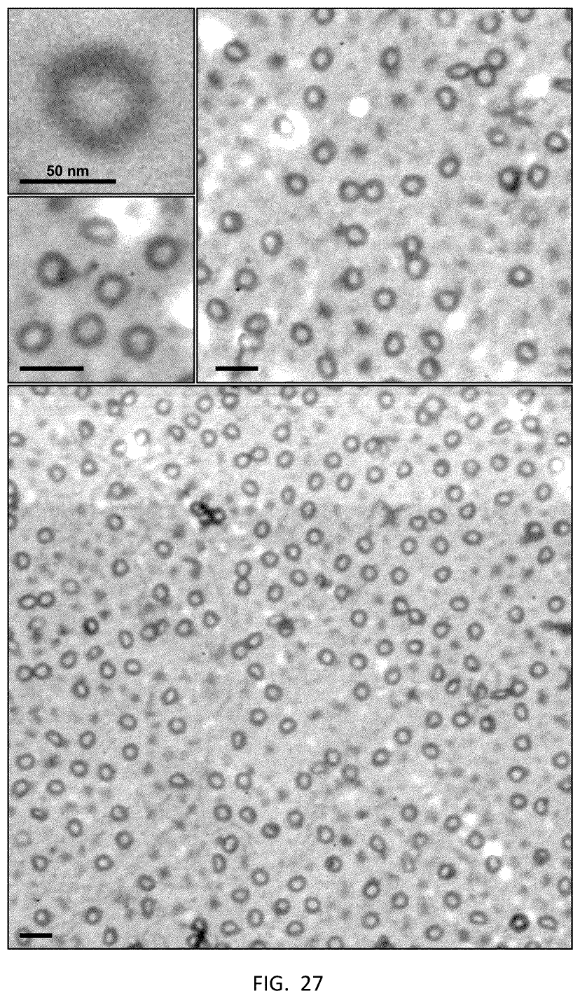

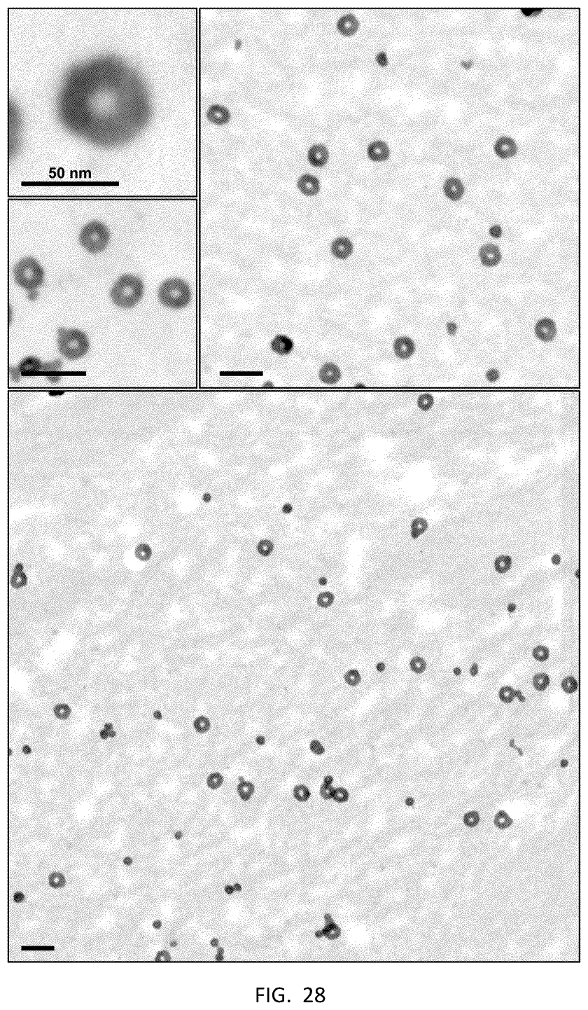

The modularity of the silica growth methods and conditions described above were assessed by applying them to a dramatically different DNA origami shape, a ring (FIG. 19F). Silica growth was performed on DNA ring-shaped DOPMs prepared using 20 kDa PEG chain length in 85% ethanol solution at pH 11, using 125×TMOS concentration. The reaction was incubated for 3 hours to allow the reagents to exhaust, terminating the growth process. From the electron micrographs showed that the structures across all the conditions were monodispersed and did not exhibit any form of aggregation at any step of the process (FIG. 19B-D for DNA bricks and FIG. 19G-I for DNA rings, wide-field images in FIG. 20 to FIG. 28). For the DNA brick-silica nanoparticles, the width increased by 40%, from 21.27±1.1 nm for DNAO to 30.61±2.12 nm for silica structures (FIG. 19E), while the length decreased by 8%, from 57.15±0.83 nm for DNAO to 54.12±1.85 nm for silica structures. Similarly, the pore diameter of DNA ring structures decreased by 59%, from 31.2±2.415 nm to 12.85±1.69 nm (FIG. 19J), while the outer diameter decreased by 3.6%, from 51.86 nm for DNAO to 49.97 nm for silica structures. These results show consistent material growth of 4.12 nm thickness for DNA brick and 4.67 nm for DNA ring.

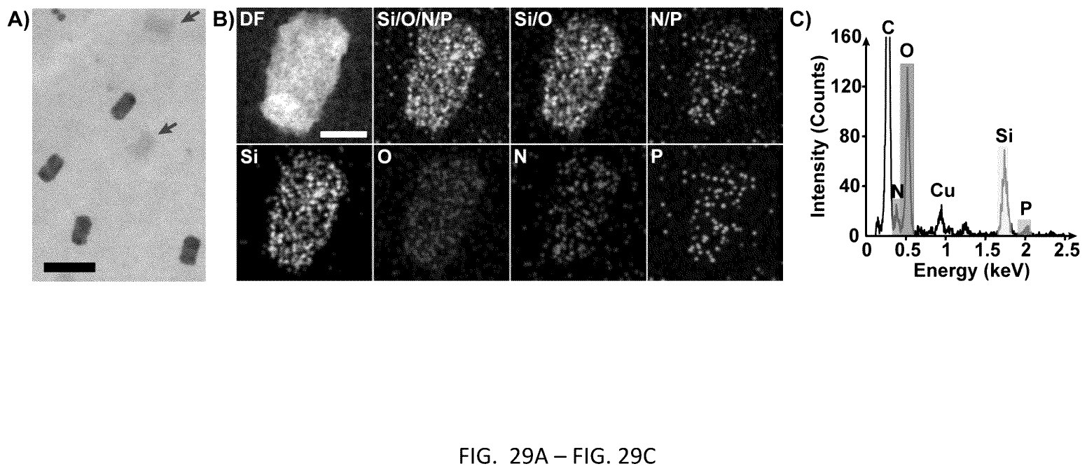

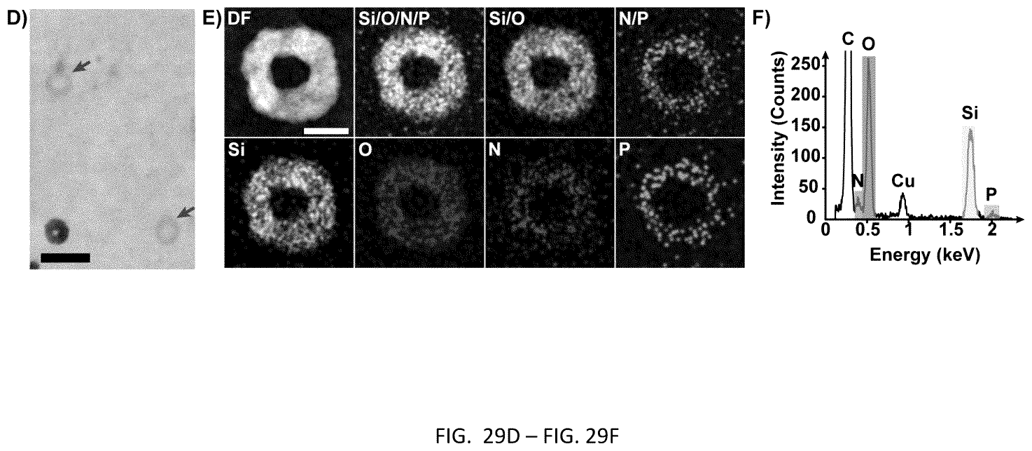

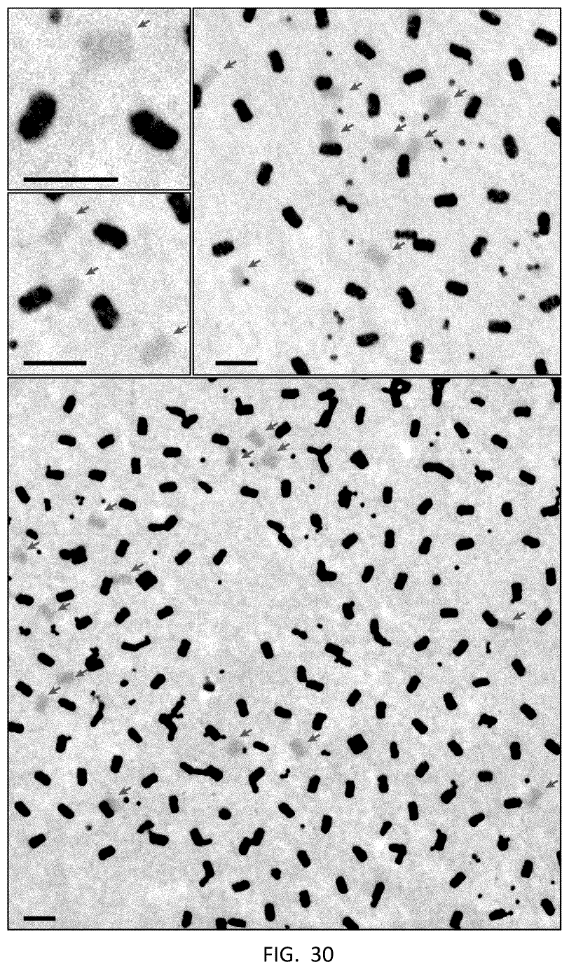

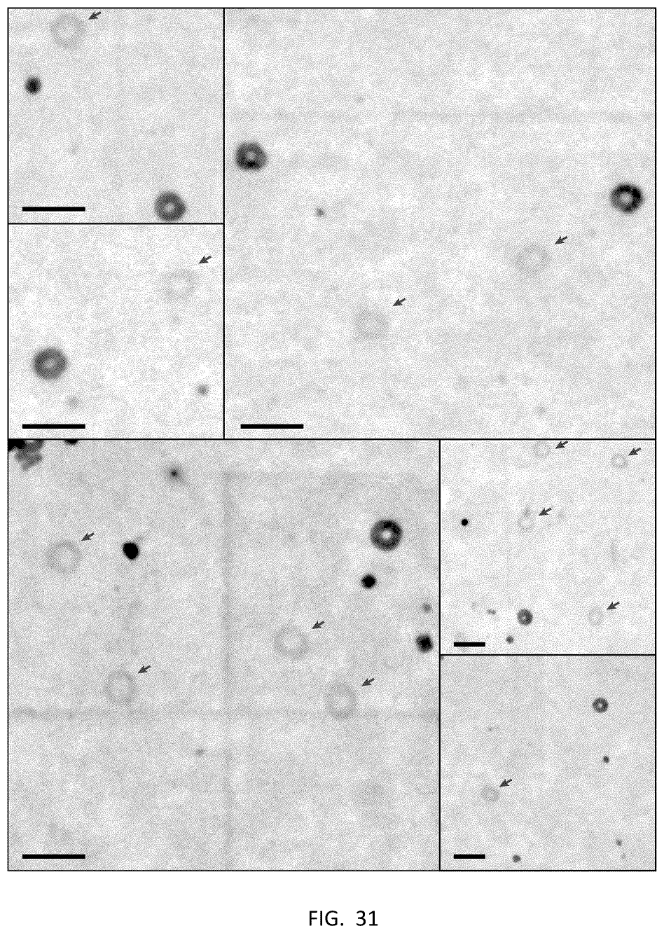

Control DNAO and DOPMS were stained with uranyl formate to allow better contrast in the resulting images. A DNA staining process for the silica-coated structure was not conducted. However, the difference in contrast and edge clarity of the structures provided a clear indication of material growth. DNAO structures were also imaged and the structures with the silica shells on the same TEM grid, and we observed a clear difference in material-related contrast due to the silica shell (FIG. 29 A, D and supporting FIG. 30 and FIG. 31).

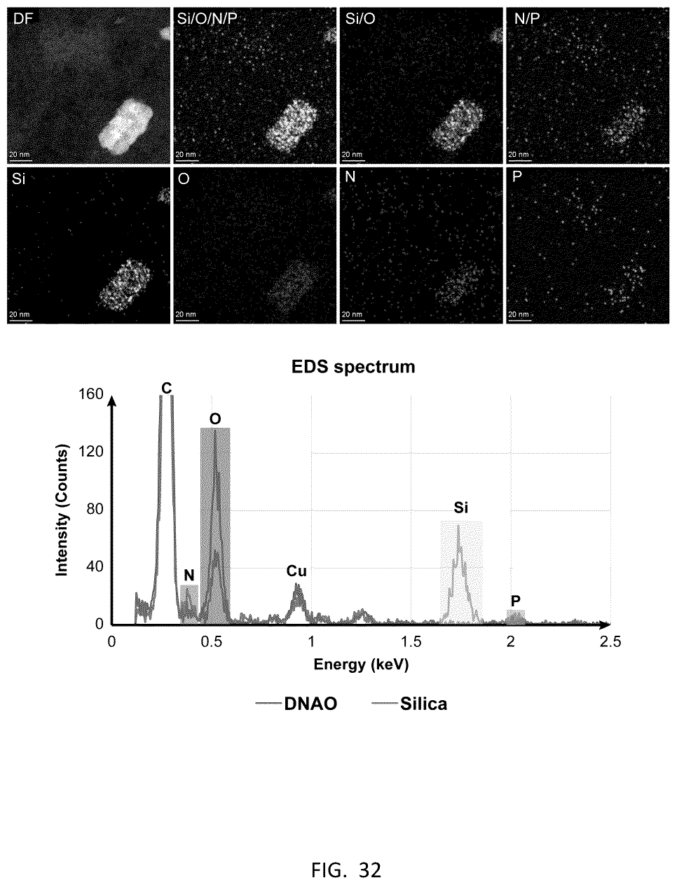

In addition, elemental analysis was performed and the energy dispersive spectra (EDS) were plotted for both nanoparticles (FIG. 29B-C, FIG. 29E-F). This analysis provided clear, unambiguous evidence of the presence of silica around the DNAO. The overlay of the dark field image of the structure with the energy signal from the constituent elements (silicon, oxygen, nitrogen, and phosphorous) confirmed that the silica growth takes place on the DNA origami structures. The dense silicon elemental map (Labeled “Si”) for the DNA brick and ring structures suggested consistent growth. A comparison study between the control DNAO and silica-coated structures on the same grid is shown in FIG. 32, in which the DNAO structures lacked the silicon intensity peak along with the decreased peak for oxygen.

### Example 10: Robustness of Silica Coated DNAO Nanoparticles

For the entire duration of the tests and optimizations, the silica-templated DNAO nanoparticles exhibited stability for long durations (over 4 days, data not included) in salt-free, aqueous, and organic solvents, where typically, native DNAO nanoparticles either degrade or aggregate. These structures also survived high pH solutions (pH 11) and pH transitions from pH 11 to 7. Thus, the structures may sustain a wide range of working conditions, enabling various applications that do not favor aqueous saline solutions.

### Example 11: Thermal Stability of Silica Coated DNAO Nanoparticles

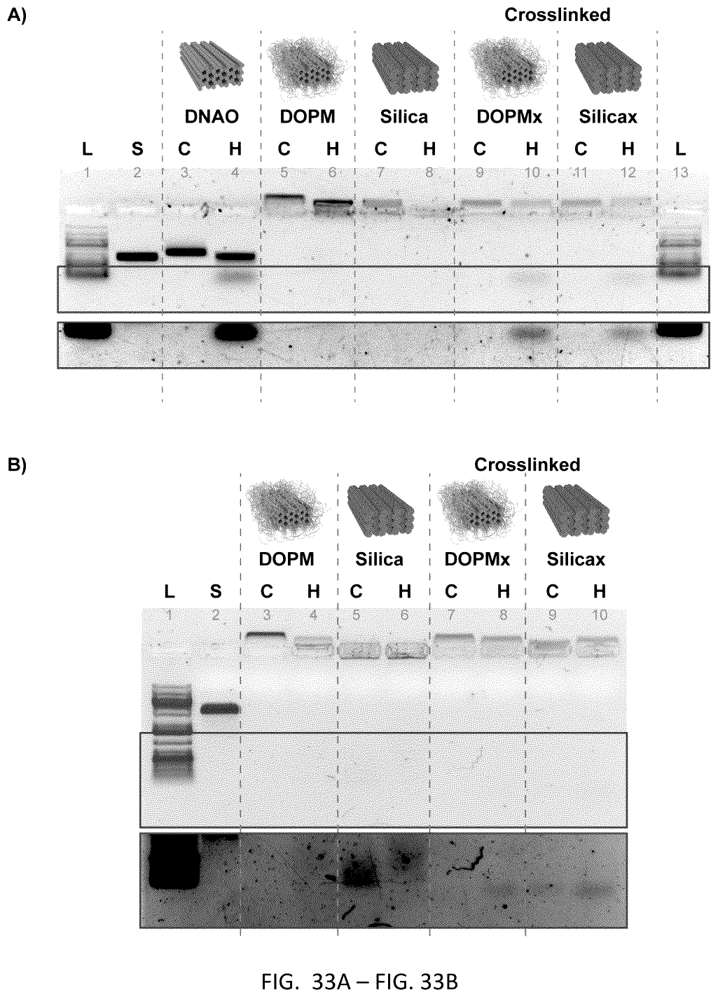

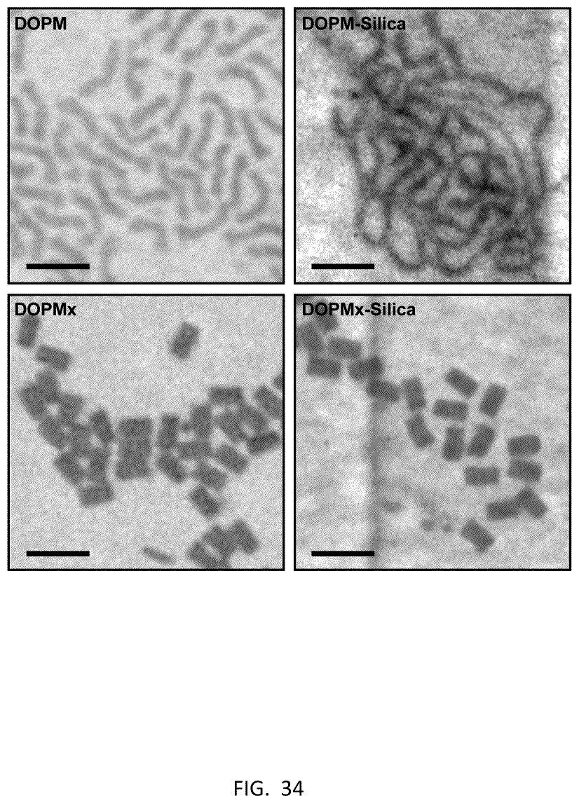

The robustness of silica DNA nanoparticles at elevated temperatures was evaluated. Typically, most native DNA nanoparticles disintegrate at temperatures above 50° C. in favorable buffer conditions, or room temperature in low-salt or no-salt aqueous buffers. Such lability presents a major challenge for solution-based applications that require elevated working temperatures. To investigate the thermal stability of the silica-coated DNA nanoparticles, silica shell growth on 42HB DOPMs using 5 kDa PEG length, 125×TMOS as a precursor in 85% ethanol at pH 11 was performed. After 5 minutes of growth, the reaction was stopped using PS, followed by the addition of HCl after an additional 15 minutes of incubation. These structures were incubated along with native structures and DOPMs as controls in a thermocycler for 30 minutes at 90° C. Upon AGE analysis (FIG. 33), it was observed that the structures did not exhibit any staple leakage. However, from the TEM images (FIG. 34), the structures appeared to have lost their shape and resembled rod-like structures (FIG. 34, top left and right panels). Both DOPMs and the structures with silica shells demonstrated similar behavior. Despite staple retention, there appeared to be a loss of structural integrity. Not to be bound by theory, but the observed thermal degradation is likely due to the condensation forces exerted by the 10-unit long polylysines on the DNA being the most prominent component at elevated temperatures, including for the silica coated structure since the silica shell is thin and likely highly porous. The lack of staple strand leakage suggested that despite the structural distortion, the strong electrostatic attractions of the polylysines retained all the staples strand within the structure. The same behavior is observed with DOPMs (Data not shown). To increase the thermal stability of the templated silica nanoparticles, glutaraldehyde crosslinking was utilized (that interconnects primary amines on the polylysines) to reduce the DNA condensation forces and improve the thermal stability. Upon repeating the thermostability test with crosslinked DOPMs (DOPMx), it was observed that despite some staple leakage, the structures maintained the shape (FIG. 33 and FIG. 34). The reduction in the overall positive charge on the polylysines appears to have led to a significant reduction in the condensation forces. To circumvent the observed staple leakage, minor design modifications were incorporated by removing weakly bound staples (staples with domain length of ≤7), which completely stopped the staple strand leakage.

### Example 12: Accessibility of Functional Groups after Silica Growth

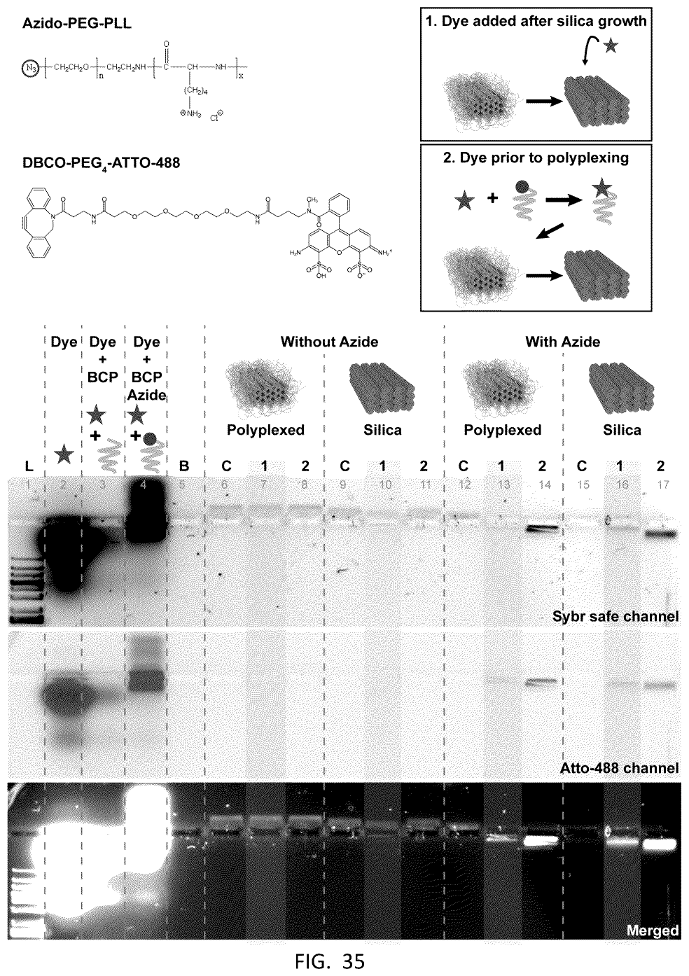

One of the primary goals of this work was to not only demonstrate templated growth of silica on DNA origami but to also demonstrate the retention of spatial programmability of the DNA origami. An an initial demonstration of this capability, a bulk accessibility test was performed. Azido-modified PEG-PLL (—N₃ groups on the terminal ends of PEG) to form the DOPMs were utilized. The presence of this terminal group enables click-chemistry to a dibenzylcyclooctyne (DBCO) functionalized fluorescent dye. For this experiment block copolymers with azido group (PLL-PEG-N3) were used, and, as a control, block copolymers without the azido groups were used (PLL-PEG). For fluorescent tagging of the azido groups, DBCO modified Atto-488 dyes were added to the reaction mixture in two separate ways: (1) The dye was added to the block copolymer prior to the polyplex micellization step, which served as the dye control set; and (2) the dye was added after silica growth. The two sets can be compared to confirm the accessibility. To facilitate the comparison, agarose gels were scanned for fluorescent signal from the dye using the blue channel, followed by post-staining with Sybr safe DNA stain to observe the fluorescence from the DNA bands. A merged version of the two fluorescent images can be observed in the bottom of the figure, with red fluorescent signal from the dye (“with azide”, lanes 1 and 2 for each construct), while grey bands depict the fluorescence from the DNA stain (“without azide” constructs; FIG. 35).

These azido-DOPMs were used for silica shell growth using 125×TMOS, 85% ethanol at pH 11, with and PS, HCl combination to stop the growth reaction. To assess the accessibility of the azido group after the growth, DBCO-functionalized Atto-488 fluorescent dye was added. The Atto-488 fluorescence from the AGE results were observed, and that the terminal groups were accessible after the silica growth was confirmed, indicating that the silica deposition occurs directly on the surface of the DNA and does not embed the PEG chains in the process. This result also indicated that by incorporating PEG chains with terminal reactive groups, at select sites on the DNA, defined reaction sites on the silica-templated nanoparticle can be created.

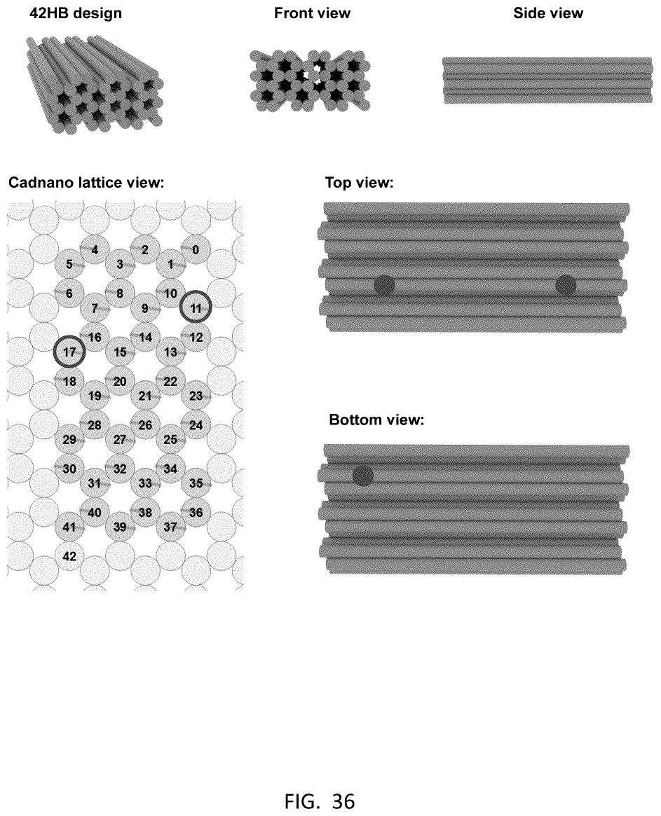

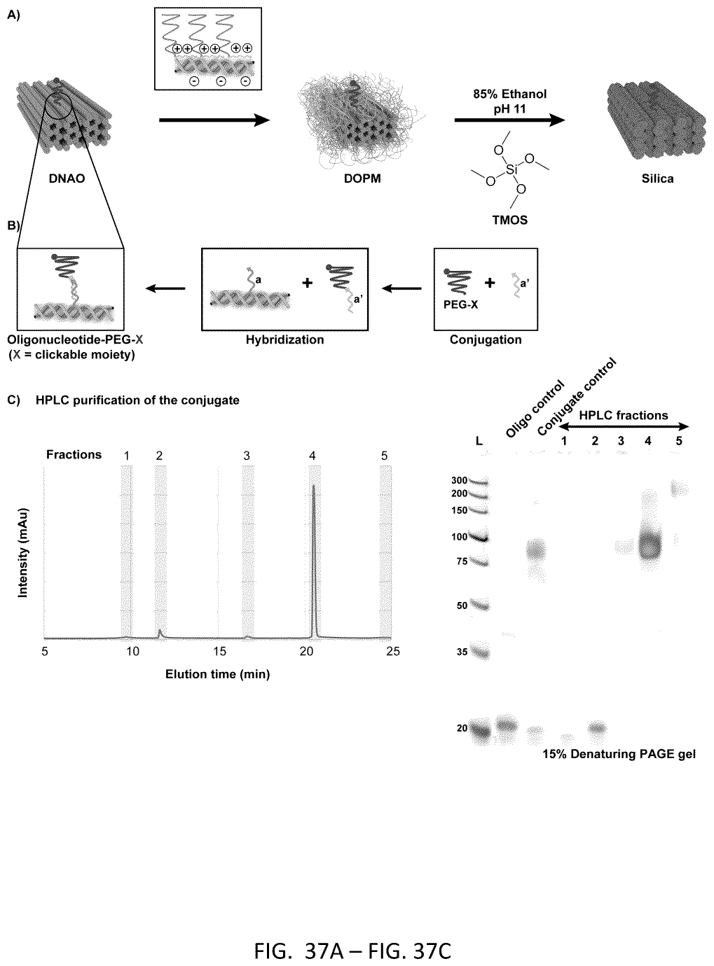

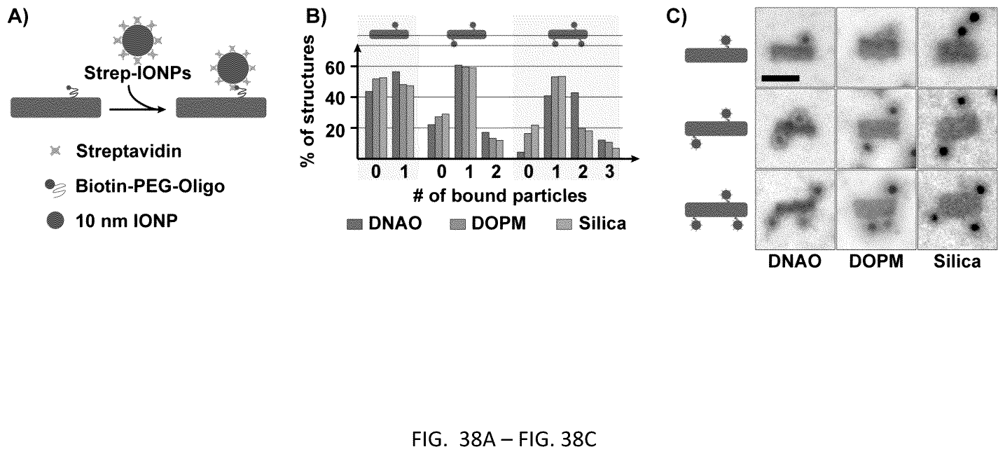

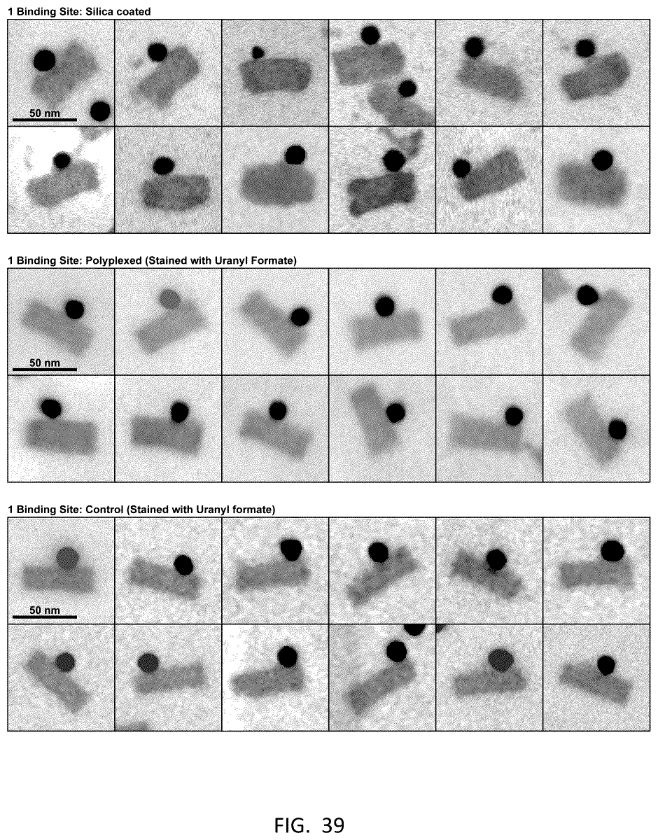

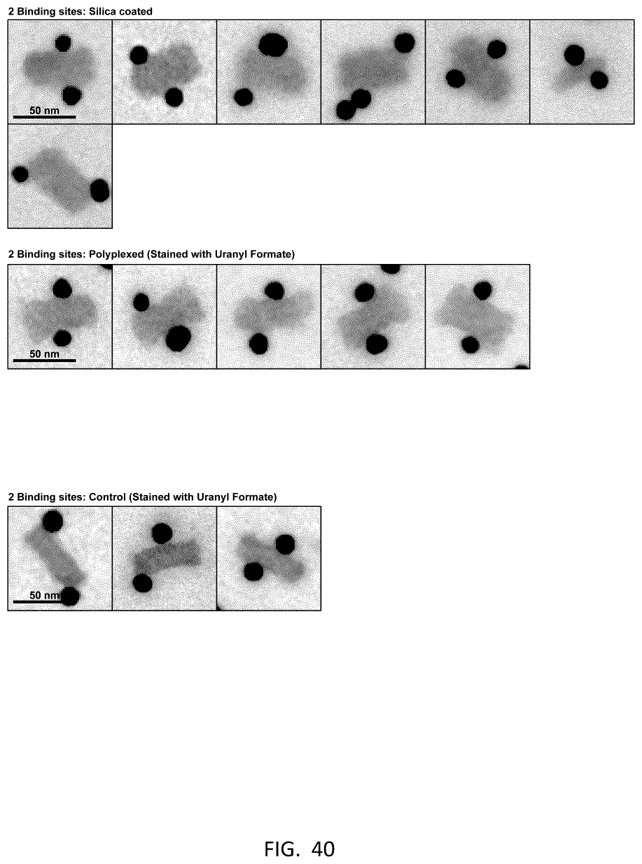

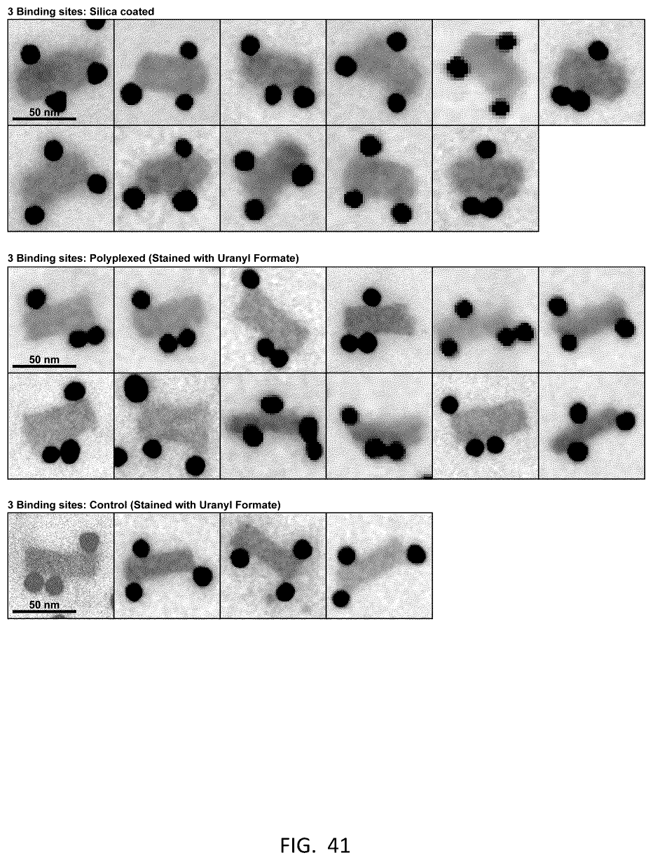

The DNA brick design was modified to accommodate 1-, 2-, or 3-single-stranded DNA extensions with unique sequences at select locations on the outer surface of the structure (FIG. 36). Next to these extensions complementary oligonucleotides conjugated with 5 kDa long PEG chains carrying a biotin group at the terminal position were added. This conjugate can bind to entities carrying streptavidin. A detailed protocol for the synthesis, and purification, of the conjugate, is illustrated in FIG. 37. These DNA brick structures with either 1, 2, or 3-biotin groups (FIG. 38A) were used to grow silica shells and evaluated the accessibility by adding streptavidin-functionalized 10 nm iron oxide nanoparticles (Strep-IONPs). Silica growth was performed by forming DOPMs with the 5 kDa PEG chain length, using 200×TMOS as the precursor, and the growth reaction was conducted for 3 hours. The resulting structures were characterized with TEM, and, for reference, native DNA nanoparticles and DOPMs without silica shells were used. From the TEM images, we confirmed the accessibility of biotin groups across the controls and the silica-coated structures (FIG. 38C, and FIGS. 39-41). Further, the interaction between the nanoparticle and the silica-templated DNA origami was observed to be statistically identical to that between the nanoparticle and DNA origami and DNA origami polyplex (FIG. 38B-C), which indicates that while the interaction between the nanoparticle and the terminal reactive group may be improved, the silica growth process does not adversely affect the accessibility of the reactive groups organized on the DNA nanostructure.

Sequencing

### Example 13: Thermostable, Enzyme-Stable DNA Nanoparticle Carrying a Single Unit of a Functional Group

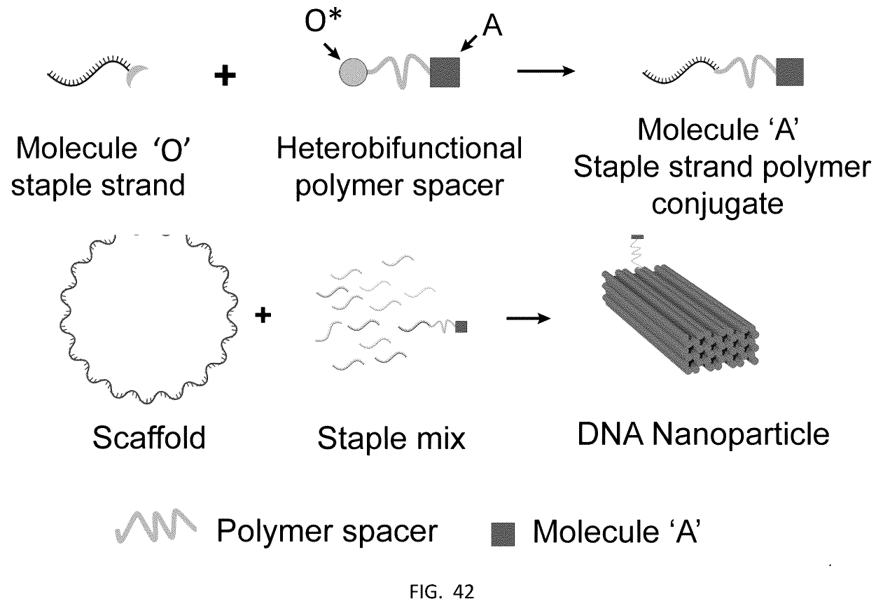

To create thermostable and enzyme-stable DNA origami, established protocols were used (FIG. 42) to synthesize a desired shape of origami. Most any shape may be suitable. The exemplified origami is designed to accommodate a single unit of two distinct functional groups, ‘A’ (shown) and ‘B’ (not shown), that are incorporated at a distinct location on the structure, functional groups (or terminal functional groups) including thiol, azide, amine, alkene, or carboxyl. To prevent cross-reactivity, the reactive groups can be separated from the DNA origami surface using an uncharged, flexible polymer spacer, such as polyethylene glycol (PEG). PEG is a water-soluble polymer commonly used in many applications, including as a spacer to separate functional groups from the surface of a biomolecule. To generate staple strands with a functional group, two of the staple strands from the desired location are modified with a functional group ‘O’. In separate reactions, a heterobifunctional polymer, such as PEG, is added to the modified staple strands. One end of the heterobifunctional polymer can bind to the staple strand ‘O’ via a complementary moiety ‘O*’, and the other is reserved for the functional group ‘A’ or ‘B’ for the next steps. This conjugate is purified, e.g., using high-performance liquid chromatography (HPLC) techniques and added to the staple mixture that forms the DNA origami. After synthesis, the DNA origami is purified to remove any remaining staple strands.

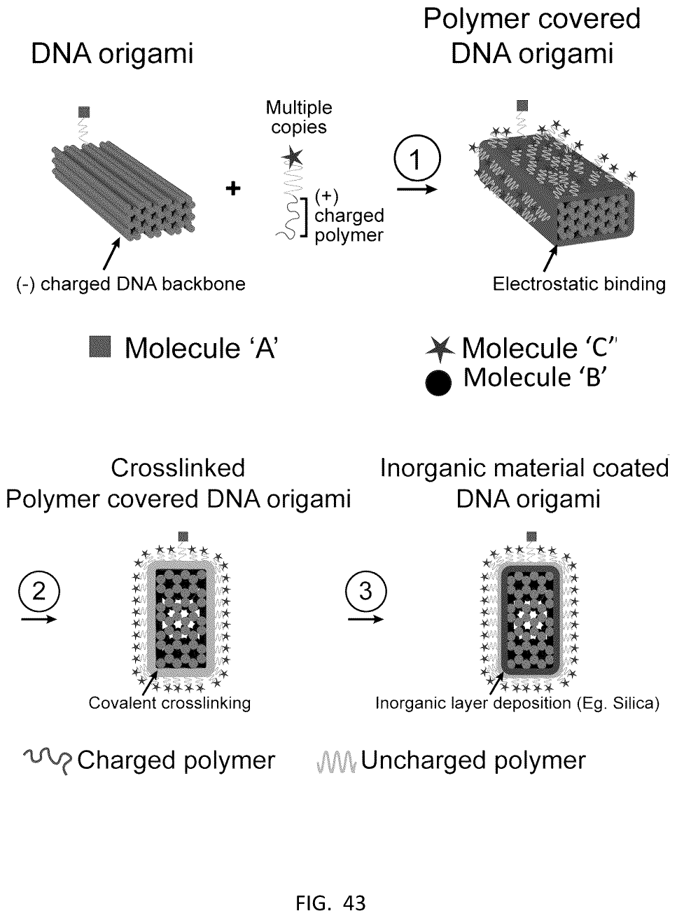

The purified DNA origami carrying the functional group is then mixed with, e.g., a di- or tri-block copolymer composed of a non-ionic amphiphilic polymer (such as PEG), a charged polymer (such as poly-L-lysine (PLL) or polyacrylic acid (PAA)) and/or a thermo-responsive group (such as poly N-isopropyl acrylamide (PNIPAm), or poly(2-Oxazoline) (POx)). The block copolymer also consists of the reactive group ‘C’ on the PEG terminus (FIG. 43, step 1). The electrostatic interaction between the charged section of the polymer and the DNA origami leads to the formation of DNA origami polyplex micelles (DOPM).

In case the charged section of the diblock copolymer is positive, the precise cation concentration in the solution is not important. However, if the charged section of the diblock copolymer is negative, the cations in the solution can be, e.g., divalent or trivalent to enable the formation of a cationic sandwich between the DNA origami and the diblock copolymer.

Such DOPMs are enzyme-stable. Mechanical stability can be further enhanced by crosslinking the polymer using appropriate crosslinking agents, such as glutaraldehyde, bis(sulfosuccinimidyl)suberate (BS3), or dimethyl 3-3′, dithiobispropionimidate (DTBP), resulting in a thin polymer matrix around the DNA origami (FIG. 43, step 2). The crosslinking process chemically binds two or more polymer chains to form a network structure. This process can improve the mechanical properties and stability of DOPMs. These crosslinked DOPMs can be used as templates to grow inorganic materials such as silica. Optimized material growth conditions are used to grow silica shells while retaining the accessibility of the functional groups (FIG. 43, step 3). These silica-coated DNA nanoparticles are thermostable.

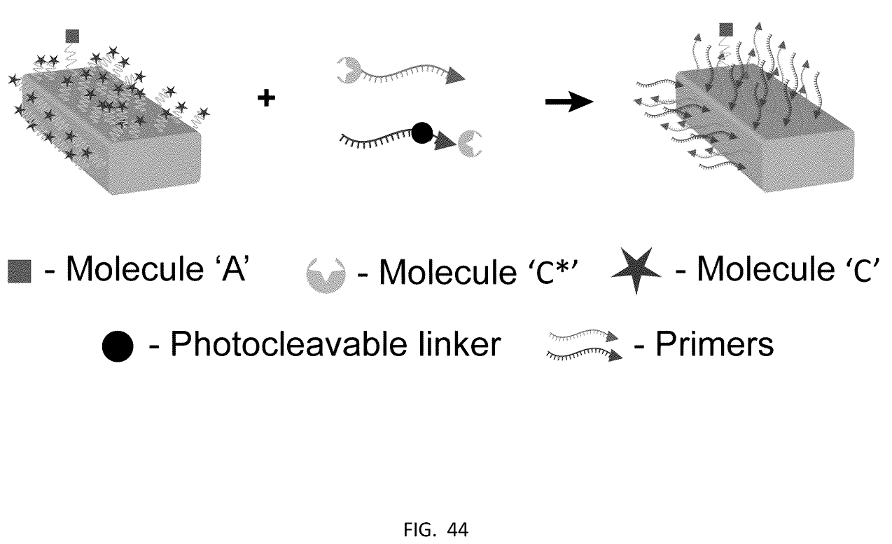

Finally, a pair of primers are bound onto the reactive group ‘C’ on the PEG terminus of the diblock copolymer, resulting in a DNA nanoparticle that has a functional group ‘A’ along with a layer of primer pairs (FIG. 44; ‘B’ not shown). One of the primers has a photocleavable linker, which allows for its removal upon irradiation with light of appropriate wavelength.

### Example 14: Target DNA Fragment Capture and Amplification

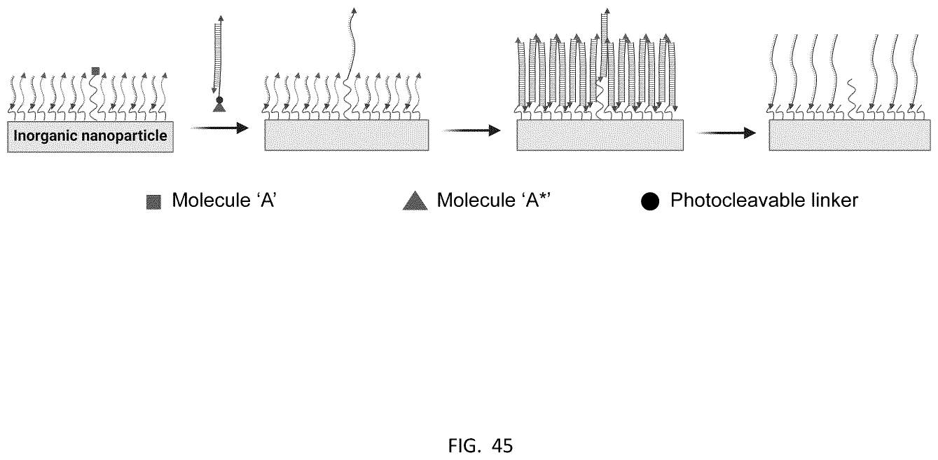

FIG. 45 illustrates a method for isolating a single DNA fragment and amplifying it locally to form a monoclonal cluster on the DNA nanoparticle. Firstly, during the library preparation step, the DNA fragments are ligated with adaptor strands on both ends, which include the primer binding region. One of the adaptors can have a functional group (here, labeled as ‘A*’) attached to it, which enables covalent bonding to the reactive group ‘A’ on the DNA nanoparticle.

DNA fragments are then incubated with the DNA nanoparticles in a denaturing condition (such as elevated temperatures, pure water, 7M urea, and the like) to minimize the interaction between the primer binding region on the fragments and the primers on the DNA nanoparticle while preserving the interaction between ‘A*’ and ‘A’. After the DNA fragments have been tethered to the nanoparticles, the excess untethered fragments are filtered out, and the solution is buffer exchanged into a non-denaturing buffer.

Finally, polymerase (such as Taq or any other high-fidelity polymerase) and dNTPs are added to the solution before thermocycling to amplify the DNA fragment on the DNA nanoparticle. The concentration and the thermocycling conditions are adjusted to minimize interaction between the DNA nanoparticles.

Functional group ‘B’ (not shown) can be used, e.g., to immobilize, capture, or otherwise bind the monoclonal polony to a surface, e.g., to a patterned or unpatterned flow cell.

Conjugates

### Example 15: Magnetic DNA-Nanoparticle Conjugate Functionalized with a Single Capture Group

Nanoparticles with specific functionality, like responsiveness to magnetic fields, can be functionalized with one or more single stranded DNA scaffold strands. The ssDNA nanoparticles are used to create DNAO-nanoparticles (DNAO-NPs) by self-assembly via an annealing incubation with the single stranded scaffold and staple strands. The addition of the scaffold strand results in each nanoparticle being functionalized with uniquely addressable sites based on the local scaffold sequence. The staple strands with complementary sequences selectively bind to these sites and can be conjugated with functional molecules. This specificity allows each nanoparticle to be functionalized with a single capture group, or binding functional moiety, such as an antibody. The result guarantees that each DNAO-NP can only capture a single target molecule without a reliance on Poisson statistics and a large excess of DNAO-NPs relative to the target. To facilitate detection of the captured target, the nanoparticle can be functionalized with one or more signaling groups, or detection functional moieties. They can either be added during the initial anneal of the DNAO-NPs, or in an additional incubation step after purification of the DNAO-NPs. In the former, the functional group is directly conjugated to the staple, which is bound to the site of interest; whereas, in the latter, the staple binding to the site of interest is modified to have, e.g., a ssDNA tail, which can then be used to bind a functionalized ssDNA strand. The decision of when to add functional groups depends on their thermostability, and the number of desired units per structure. For instance, to functionalize the structure with a large quantity of signaling units, they can be incorporated during the initial anneal by using different staples functionalized with the signaling groups. However, to eliminate the need for such an abundance of different functionalized staples, the staples are instead modified with a ssDNA tail of the same sequence. This facilitates incorporation of the signaling group via a single complementary functionalized ssDNA in a second incubation step. After each synthesis or modification step, the DNAO-NPs can be purified via several methods, including centrifuge-based pelleting purification after the initial anneal to isolate the product from excess staple strands and DNA structures that have formed without the nanoparticle core or, alternatively or in addition to, purified via a filter-based method.

### Example 16: Capture and Detection of the Targets

Targets of interest are captured in solution phase by incubating a sample containing the targets with a moderate excess of DNAO-NPs. This solution is then incubated in a flow chamber with a substrate functionalized with a secondary capture group to immobilize the captured targets to the substrate. Since this immobilization is mediated by the target itself, DNAO-NPs that have not captured targets do not bind to the substrate. For magnetic nanoparticle embodiments, the process can be expedited by applying a magnetic field to pull all DNAO-NPs to the surface of the substrate. Since each structure has at most a single target, the magnetic field is removed to facilitate immobilization by allowing the structures to re-orient to increase the targets' access to the substrate. The magnetic field is then reversed to pull DNAO-NPs that have not captured targets away from the substrate. These excess structures are then washed away prior to detection of the targets.

The flow chamber can be as simple of a construct as a glass coverslip, glass slide, and double sided tape. Fluid exchange is achieved by simultaneously wicking the chamber and flowing in fluid. In such a flow chamber, the chamber surfaces are prepared by, e.g., cleaning the glass surfaces with ultrapure water and isopropyl alcohol. Double sided tape is applied to the coverslip to set the chamber dimensions, and the coverslip is plasma activated to further clean the surface and enhance its adhesive properties. After the chamber is assembled by attaching the coverslip and the glass slide, the coverslip can be functionalized. Functionalization of the coverslip with secondary antibodies can be achieved, e.g., by adsorption or covalent immobilization strategies using glass modifications including poly-L-lysine, silane, and epoxy. To reduce nonspecific binding of the DNAO-NPs, the surface can be pre-blocked or blocked during target immobilization with biomolecular or non-ionic detergent blockers including BSA, sheared salmon sperm DNA, and Tween20.

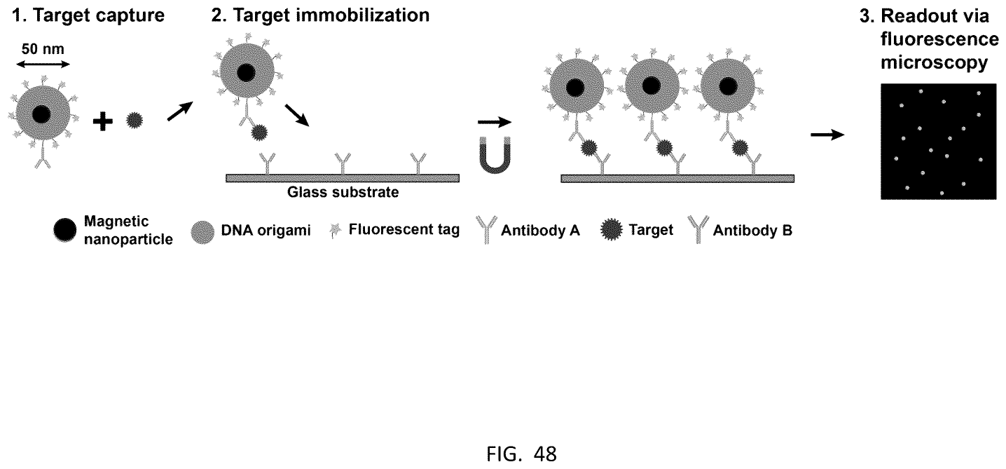

For embodiments with fluorescence-based detection functional groups, detection of the targets can be conducted dry or in solution via fluorescence microscopy. To reduce bleaching of the fluorophores imaged in solution, oxygen scavenging techniques can be employed. Each target appears as a bright point on the surface. The surface is then imaged over several areas, and the images are analyzed to produce targets counts or densities by counting the number of bright points. To set a standard curve, dilutions of known target concentrations are analyzed. This allows samples of unknown concentration to be quantified via comparison with the curve. Since this method is intended to quantify targets at low concentration ranges, the bright points are well separated, and each point has a low probability of being produced by multiple captured targets. To adapt this method to quantify higher-concentration samples, the samples can be diluted to lower concentrations sufficient to result in well separated points (FIG. 48).

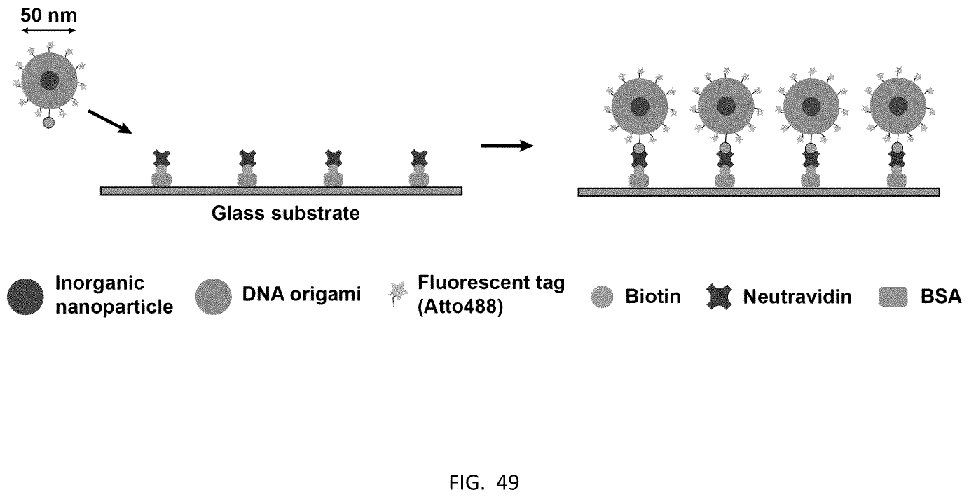

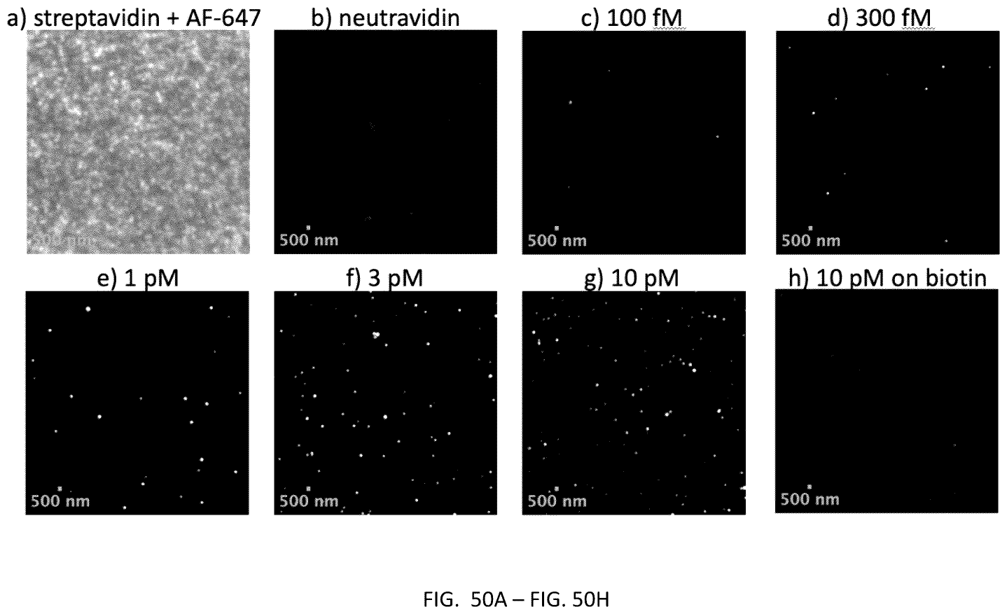

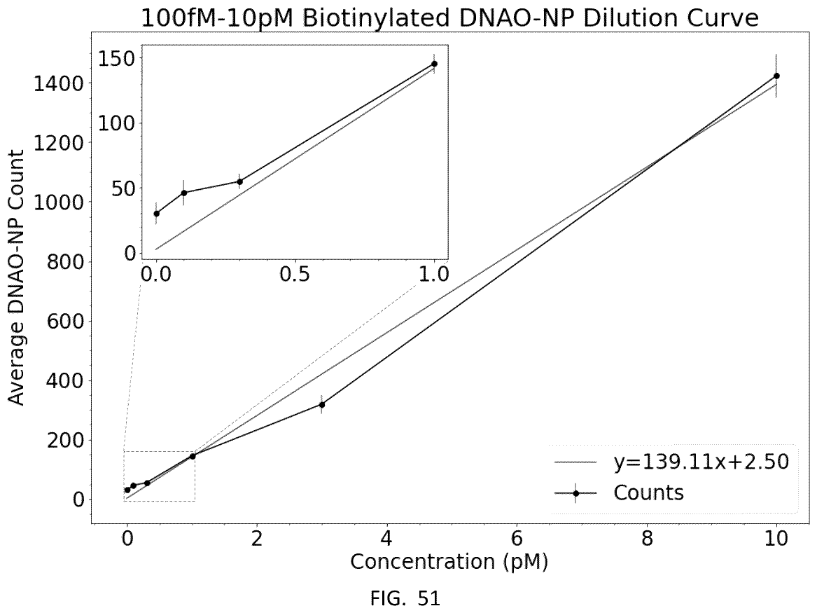

To verify the detection and counting of DNAO-NPs, non-magnetic, biotinylated fluorescent DNAO-NPs with a gold core were immobilized on a surface covered with neutravidin, dried, imaged via fluorescence microscopy, and counted. FIG. 49 illustrates the experimental setup and FIG. 50 shows images of a variety of dried samples of varying concentrations of fluorescent biotinylated DNAO-NPs immobilized on a biotin-BSA surface. To first verify formation of the neutravidin surface, an alternate form, streptavidin conjugated with Alexa Fluor 647, was bound onto a glass surface coated with biotin-BSA. Biotinylated DNAO-NPs were immobilized on a neutravidin surface at concentrations between 100 fM and 10 pM, imaged, and then analyzed with FIJI (FIG. 51). To assess the background levels, neutravidin and biotinylated DNAO-NPs on a biotin surface were examined.

### Example 17: DNA Origami Nanoparticles (DNAO-NPs) and Dimerization

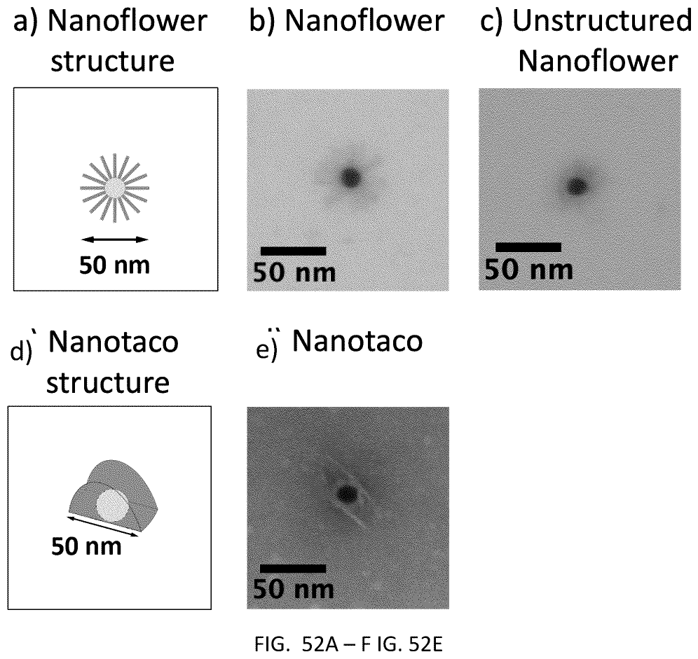

One quantification method utilizes an inorganic nanoparticle coated in ssDNA and conjugated to a single scaffolded DNA origami to achieve monofunctionality. Such structures include the nanoflower, which is known in the art, as well as the nanotaco described herein. TEM images of these structures can be structure seen in FIG. 52B, C, and D. The Nanotaco is composed of a disc-shaped origami wrapped around an inorganic nanoparticle.

Dimerization

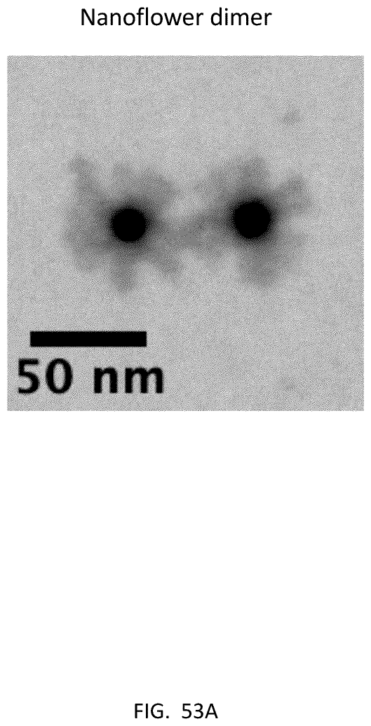

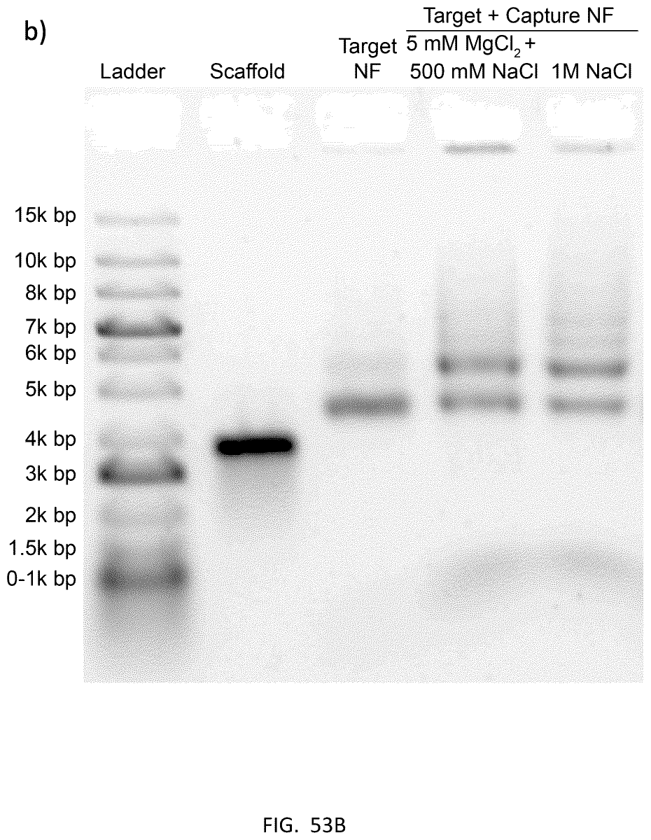

To assess the functionalization of the DNAO-NPs, nanoflower dimers were formed via ssDNA hybridization and annealed at a 1:1 ratio with a 1 hour linear ramp from 44° C. to 30° C. at −1° C./4 minutes. These annealing conditions primarily form dimers, but can also result in aggregates. The structures were analyzed via TEM (FIG. 53A) and agarose gels (FIG. 53B).

Other embodiments include the use of, e.g., magnetic biotinylated DNAO-NP, as well as quantification of a protein with both magnetic and nonmagnetic DNAO-NPs. Although this work was conducted using fluorescent signals and read out via fluorescent microscopy, the method is adaptable to other signaling and readout methods by modifying the detection groups the DNAO-NPs are functionalized with. For example, the signaling mechanism could be electrochemical, enzymatic, or nucleic acid-based. Lastly, to add robustness to solution conditions, a polyplex micelle shell can be added to the DNAO-NPs.

Methods

DNA Origami Design.

The DNA brick (42HBs) design that has been described previously by Engelhardt, et al., was used without any design modification (sequences and the caDNAno design occurs as 42HB_staples.xls and 42HB.json, as a part of the zip archive 42HBdesgin.zip). Briefly, the design consists of a total of 213 oligonucleotides (staple strands), distributed across three 96-well plates at a concentration of 200 pM each. These oligonucleotides were pooled to a stock solution and diluted to a working concentration of 800 nM each. The design was modified to accommodate staple extensions at three locations as described in the FIG. 36. These extensions were 20 bases long and had the following sequence: CAACCTCATACCACTACAAC (SEQ ID NO:1), while the complementary oligonucleotide had the following sequence: GTTGTAGTGGTATGAGGTTG (SEQ ID NO:2).

The DNA ring (delftagon) design described previously by Fragasso, et al., was used without any design modification (sequences and the caDNAno design occurs as ring staples.xls and ring.json, as a part of the zip archive Ringdesign.zip). Briefly, the design consists of a total of 240 staple strands, distributed across three 96-well plates at a concentration of 200 pM each. These oligonucleotides were pooled to a stock solution and diluted to a working concentration of 1600 nM each.

DNA Origami Folding.

In the folding reaction for DNA brick, 20 nM of p7560 scaffold (Tilibit nanosystems GmbH, Germany), respective staple strand set (Integrated DNA Technologies, IDT) at 100 nM (each staple, a 5:1 staple to scaffold ratio), in 1×Tris EDTA buffer, pH 8.0 at 25° C. (Sigma Aldrich, 10 mM Tris, 1 mM EDTA) with 15 mM magnesium chloride (Sigma Aldrich) were mixed. The mixture was annealed in a thermal cycler (Bio-Rad C1000 Touch) for 65° C. for 15 min for denaturation, followed by a folding ramp from 60 to 20° C. at the rate of −1° C. per 3 min and held at 20° C. for storage.

For the DNA ring, the reaction mixture consisted of 10 nM of p7560 scaffold, staple mix at 300 nM (30:1 ratio), in 1×Tris EDTA buffer with 24 mM magnesium chloride. The mixture was annealed for 65° C. for 15 min for denaturation, followed by a 16-hour hold at 50° C. The folded structures were stored at 20° C. until further purification.

Excess staple strands were then removed by ultrafiltration columns (50 kDa MWCO Amicon Ultra-0.5 Centrifugal Filter unit, AMD Millipore).

Purification with Ultrafiltration Columns.

Passivation. Prior to purification, the columns were passivated by adding 500 μL of 5% Pluronic F-127 (Sigma Aldrich) solution to the filters and incubating for 30 min at room temperature. After the incubation, the filters were flipped to discard the solution and spun in the flipped state for 1 min at 2,000 g to remove any remaining solution. The filters were then washed with 500 μL of ultrapure water and flipped to discard the solution. This step was repeated for a total of four times, after which the filter was used right away as leaving the filter unused or without any solution for a long duration as the filter membrane can dry out, leading to poor yields. In addition, avoid letting the Pluronic solution pass through the filter, as it will block the pore completely.

Purification. To the passivated filter, the crude DNAO mixture was added along with the wash buffer (1×Tris EDTA, pH 8.0 with 5 mM MgCl2) to bring the total volume to 400 μL. The filter was spun at 14,000 g for 2 min at room temperature, the flow through was discarded and 400 μL of the wash buffer was added. The total washes were calculated as per the total staple concentration. Typically, 8 washes were performed for 200 μL of the impure reaction mixture consisting of 100 nM of each staple. The last wash step was performed for 5 min to obtain a concentrated purified mixture. After the final wash the filter was reversed, placed in a fresh tube, and centrifuged at 1,000 rcf for 2 min. The purified DNA origami solution was then collected for further characterization and reactions.

Preferably, add the wash buffer prior to the addition of the crude mixture, e.g., if the DNAO mixture is 40 μL, add 360 μL of the wash buffer to the filter followed by the DNA mixture. In addition, adding less than 50 fmoles of total staple concentration in the filter avoids poor purification yields.

Preparation of DOPMs.

The PEG-PLys with varying PEG chain lengths (1, 5 or 20 kDa) or the terminal azide group (N₃—PEG-PLys with 5 kDa PEG chain length) were purchased from Alamanda Polymers Inc. It is composed of a PEG segment and a cationic PLys segment with DP 10.

DNA Origami Polyplex Micelle Preparation.

Standard preparation. For the preparation of DOPMs, the polyplex micellization strategy was used as described previously by Agarwal, et al. (DOI:10.1002/anie.201608873). The block copolymer solution was added at the desired N/P ratio to DNA origami solution and the mixture was immediately vortexed. The required volume of PEG-PLys needed for DOPM formation was calculated using the excel calculator (provided in the supporting files for Agarwal, et al.). After a brief incubation the DOPM solution was purified to remove excess PEG-PLys, and buffer exchanged to ultrapure water.

Purification of DOPMs Using Ultrafiltration Columns.

For purifying the mixture after complexation and to remove excess PEG-PLys, the passivation and purification protocol described above were used. For purification, 400 μl of ultrapure water were used instead and a total of 4 washes were performed to remove salts. After the final wash, the filters were reversed, placed in a fresh tube, and centrifuged at 1,000 rcf for 2 min. The purified DOPMs solution was then collected and used further.

Glutaraldehyde Crosslinking of Polylysines

Purified DOPMs in ultrapure water were used for glutaraldehyde crosslinking. For this the N/P ratio was assumed to be 2 after purification and accordingly, 200 times glutaraldehyde (50% in water, Grade 1 for electron microscopy, Sigma Aldrich) per amine was added to the DOPMs and mixed thoroughly. The reaction mixture was incubated at room temperature overnight. The crosslinked DOPMs (DOPMx) were purified using Pluronic passivated 50 kDa ultrafiltration columns using the protocol mentioned earlier. Briefly, three washes were performed with 450 μL ultrapure water to obtained purified DOPMx.

Silica Growth on DOPMs

Transfer to 85% ethanol, pH 11. DOPMs in ultrapure water at high concentrations (>50 nM) were diluted to a final concentration of 2.5 nM using non-denatured ethanol (200% proof for molecular biology, Sigma Aldrich) to get the final ethanol content to 85%. Next, ammonium hydroxide (28.0-30.0% NH₃ basis, ACS reagent, Sigma Aldrich) was added to a final concentration of 0.14 M to get to pH 11. For this, 695 μL were taken in 250 μL water to get 5 M ammonium hydroxide solution. This mixture was vortexed thoroughly to obtain a homogenous mixture.

Addition of precursor. To the above mixture, precursor (TMOS, 99% Thermo Scientific; or TEOS, reagent grade 98% Sigma Aldrich) was added to initiate silica growth and mixed immediately. The mixture was incubated at room temperature. For experiments with DLS measurements, the mixture was immediately used for measurement after the precursor addition. Dilution of the precursor was performed using dimethyl sulfoxide (DMSO, 99.9% ACS reagent Sigma Aldrich). The diluted precursors were always freshly prepared and used immediately.

Calculations for the precursor concentration. From several preliminary tests (data not shown), the precursor concentration was adapted to an arbitrary number of 9,300 molecules per nm² of origami surface, which corresponded to 37.2 mM precursor per 1 nM for DNA origami. This amount is denoted as 1× precursor concentration. Any further dilution factor of the precursor is denoted with respective number, for instance 10× or 125× dilution for 10 times or 125 times dilution to the above mentioned 37.2 mM precursor concentration. The appropriate dilution factor was worked out by observing the DLS results. Further, the dilution factor can be adjusted as per the requirement of the silica coating thickness.

Variations. Several adjustments were made to the above protocols to verify the optimal growth conditions. In all the instances, the growth protocol was maintained while changing single parameters. For experiments with methanol, like ethanol, DOPMs in ultrapure water at high concentrations were diluted using pure methanol (99.9%, HPLC grade, Sigma Aldrich). For experiments requiring pH transition from pH 11 to 7, hydrochloric acid (37%, reagent grade, Sigma Aldrich) were used. Final concentration of 0.2 M of HCl were added to 100 μL of the reaction mixture and mixed thoroughly.

Stopping silica growth. For stopping the growth reaction PEG-silane (3-[Methoxy(polyethyleneoxy)6-9] propyltrimethoxysilane, tech grade, Gelest, Inc.) was used. Any necessary dilutions were performed using dimethyl sulfoxide (DMSO, 99.9% ACS reagent Sigma Aldrich). The diluted solutions were always freshly prepared and used immediately.

Purification/moving to ultrapure water. For purifying the silica structures from unreacted excess reagents, 350 μL of 85% ethanol, pH 11 wash buffer was added to 100 μL of the reaction mixture. This solution was added to Pluronic passivated 50 kDa ultrafiltration column and spun at 14,000 g for 2 min at room temperature. The flow-through was discarded and 400 μL of fresh wash buffer was added. This wash step was repeated for a total of three times followed by another three washes with 400 μL of ultrapure water for buffer exchange. After the final wash the filter was reversed, placed in a fresh tube, and centrifuged at 1,000 rcf for 2 min. The purified DNA origami solution was then collected for further characterization and reactions.

Agarose Gel Electrophoresis

For the AGE analysis, 0.3% agarose gels (Type I, Sigma Aldrich) were casted with 0.5×Tris Borate EDTA buffer (prepared used 10×Tris-borate-EDTA buffer, ultra-pure grade VWR life science) containing 12 mM MgCl₂ and pre-stained with 1×Sybr safe DNA gel stain (Invitrogen by ThermoFisher Scientific). As a running buffer, 0.5×TBE buffer with 12 mM MgCl₂ was used. For sample preparation, 15 μL of sample solution were mixed with 3 μL of 6× gel loading dye (15% Ficoll 400, 5 mM Tris, 1 mM EDTA, 12 mM MgCl₂, 0.03% bromophenol blue and 0.03% of xylene cyanol). Electrophoresis was performed at 70 V for 2 h at room temperature. As a reference, 3 μL of 1 kb plus DNA ladder (ThermoFisher Scientific) were added. The gel was imaged with the UVP Gel Studio gel scanner (Analytik Jena GmbH). For experiments without salt, the gel and the running buffers were prepared without MgCl₂.

Dynamic Light Scattering Measurements

Dynamic light scattering (DLS) is used for characterizing the size distribution profile of particles in a suspension. The principal of the technique is that the particles are in constant thermal motion, called Brownian motion, and diffusion speed of these particles is a function of their size. Smaller particles diffuse faster than larger particles. To measure the diffusion speeds, the particles are illuminated with a laser and the pattern (scattering intensity) produced is observed using a sensitive avalanche photodiode detector (APD). The change in intensity can be analyzed and the fluctuations produces a curve which is correlated with time to measure size and size distribution. The size of the particles is measured using the Stokes-Einstein equation which gives the hydrodynamic radius (or diameter, R) of the particle using the diffusion coefficient (D) according to:

$${R}_{d} = \frac{{k}_{B} T}{3 π η D}$$

Where, k_{B} is the Boltzman constant, T is the absolute temperature and η is the liquid viscosity.

DLS measures the apparent size of the dynamic hydration layer that exists around a solid spherical particle in a solution and gives in the form of Z-average (Z_{avg}). However, the assumption that particles are spherical, does not reflect the true particle size for non-spherical particles. This suggests that using a highly asymmetric structure such as a rod or a flat sheet would result in multiple peaks. In this paper, we have used the DNA brick design which as per the DLS results is assumed to be a sphere with a diameter of 52±2 nm instead of a cuboid of 55×20×10 nm dimensions. This was consistent across all the measurements and therefore adapted as control for comparison.

Another parameter that is measured is the Derived mean count rate (kcps), which is the theoretical count rate one would obtain at 100% laser power with zero attenuation, or essentially, the total number of scattered photons detected, usually stated in a per second basis while accounting for the different attenuation levels. This parameter allowed to observe the addition of inorganic material on the DNA origami as a function of time. The increase in the scattering intensity directly correlated to the increase in the inorganic material on the origami. This parameter allows tracking the material growth when the Z_{avg} stays constant up until the aggregation time point.

All the measurements were performed using the Zetasizer Ultra (Zetasizer advanced range, Malvern Panalytical). Low volume disposable cuvettes were used with adaptors to avoid any evaporation during measurements (ZEN0040 UV-Cuvette with 8.5 mm window height, BrandTech Brand). All the samples were equilibrated at 25° C. for all measurements. All the results were analyzed using the ZS xplorer software available at the manufacturer website. For all the control measurements, the size measurements were performed 10 times. For all the growth reactions, the number of repeats were set as per the desired incubation time.

tSEM Characterization

Carbon-coated TEM grids (400 mesh copper, formvar/carbon film 10 nm/l nm thick, Electron Microscopy Sciences) were plasma-treated for 15 seconds. 2 μL of the sample solution were drop casted on the grid and incubated for 5 min. The excess solution was removed from the grid with a filter paper. Next, 5 μL of a 2% uranyl formate solution was applied for 90 seconds to stain the DNA origami structures, and the solution was removed with a filter paper. Additionally, to remove excess stain, two washing steps were performed with 10 μL of ultrapure water for 10 seconds each and the water removed with a filter paper. The samples were scanned on Gemini SEM450 (Zeiss) operated at 20 kV.

Preparation of 2% uranyl formate solution. 100 mg of uranyl formate (Electron Microscopy Sciences) were added to 5 ml of boiled ultrapure water. The mixture was stirred for a minimum of 5 min and filtered using a 0.22 μm syringe filter. The filtered solution was stored at −20° C. in 100 μL aliquots. Prior to staining, 2.5 μL of 1 M NaOH solution were added to 100 μL aliquot of 2% uranyl formate and mixed at room temperature for 5 min.

Semi-Automated Size Measurements of Structures Using TEM Images

To quantitatively analyze the size of the structures imaged using transmission electron microscopy (TEM), ImageJ was used to plot the grey value profiles of the structures and then used the python script (https://github.com/ewuirl/TEM-ruler) to analyze the profiles and calculate the average size distribution. Specifically, the TEM images were imported into ImageJ and used the ROI manager to draw regions of interest (ROIs) around the structures of interest. Then, “plot profile” was selected from the ImageJ menu to obtain the grey value profiles for each ROI. Finally, the python script was used to analyze the grey value profiles and calculate the average size distribution of the structures based on the profile data.

TEM images were used to plot grey value profiles using imageJ ROI manager. The grey-values profiles were further analyzed by python script called TEM-ruler (available on GitHub) to obtain average size distribution.

Scanning Transmission Electron Microscopy and Energy-Dispersive X-Ray Spectroscopy

Scanning TEM and elemental mapping of DNAO and silica structures was performed at 200 kV using a FEI Titan Themis equipped with a SuperX EDX detector in annular dark field (ADF) mode.

PAGE Gel Analysis

Casting 15% denaturing PAGE gels. 18.2 g of Urea (BioXtra, pH 7.5-9.5 (20° C., 5 M in H2O), Sigma Aldrich) was dissolved in 20 mL of ultrapure water. To the mixture following components were added: 5 mL of 10×Tris-EDTA-Borate buffer, 18.75 mL of Acrylamide/Bis-acrylamide solution (Ambion, 19:1 40% w/v solution, Invitrogen, Thermofisher Scientific), 400 μL of freshly dissolved Ammonium persulfate, 10% w/v (for molecular biology, >98% Sigma Aldrich), 30 μL of N,N,N′,N′-Tetramethylethylenediamine (TEMED, Surecast, Invitrogen, Thermofisher Scientific). This solution was mixed by inverting the tube. Then the solution was carefully poured into empty PAGE cassettes (1.5 mm thickness, mini, ThermoFisher Scientific). The prepared volume was sufficient for 4 cassettes. Combs were carefully placed into the cassettes without spilling the solution. The cassettes were incubated at room temperature for 30 min. For storage, the cassettes were placed in a bag with 0.5×TBE buffer and placed at 4° C. until further usage.

Sample loading preparation. 5 μL of the sample were mixed with 5 μL of 2× denaturing loading solution (50% formamide, 10 mM NaOH, traces of bromophenol blue and xylene cyanol). For the ladder, 0.2 μL of Ultra low Range DNA ladder (ThermoFisher Scientific) were mixed with 0.3 μL 10× folding buffer, 5 μL of 2× denaturing loading dye and 4.7 μL of ultrapure water. The empty lanes were filled with a blank solution composed of 5 μL of 2× loading dye and 5 μL of ultrapure water.

Protocol for running the gel. 1×TBE buffer warmed to ˜65° C. was used as the running buffer. The lanes were rinsed with 1×TBE buffer to remove excess urea. The gel was pre-run at 230 V for 30 min inside a thermal box filled with hot water (˜65° C.). The lanes were rinsed again prior to loading the prepared samples. The gel was run under the same condition as mentioned before. After the run, the gel was post-stained with SYBR gold nucleic acid gel stain (10,000× in DMSO, Invitrogen, ThermoFisher Scientific). The gel staining solution was prepared by mixing 45 mL ultrapure water, 5 mL absolute ethanol. To this mixture 5 μL of the SYBR gold dye was added. The gel was imaged with the UVP Gel Studio gel scanner.

Thermal Stability Tests

For heat denaturation tests, the reaction mixture containing DNAO or DOPMs or silica structures were incubated in a thermocycler for 30 min at 90° C. After the incubation, the solutions were placed in −20° C. freezer for 10 min to stop any further degradation. The solutions were immediately used for characterization with AGE and TEM.

Preparation of Fluorescently Labelled Block Copolymers

For preparing fluorescently labelled PEG-PLys, terminal azido functionalized PEG-PLys with 5 kDa PEG chain lengths were used. As a fluorescent label, DBCO-PEG4-Atto448 (Lumiprobe) was used. The obtained powder from the manufacturer was dissolved in DMSO to obtain 10 mM stock solution, which was stored in −20° C. freezer and wrapped in a foil to avoid any light exposure.

Labelling reaction prior polyplex micellization. For the click reaction, calculations were performed to fluorescently label 50% of the PEG-PLys to avoid any additional purification steps. The reaction mixture was incubated overnight at room temperature and wrapped in a foil to avoid any light exposure. The fluorescently labelled PEG-PLys were used without any purification for DOPM formation. However, purification steps were performed to remove excess PEG-PLys and for buffer exchange to ultrapure water.

Labelling reaction after polyplex micellization or after silica growth. Similar calculations were performed with the aim to label 50% PEG-PLys on purified DOPMs or silica structures in ultrapure water. After the addition of the dye, the reaction mixture was incubated overnight at room temperature. The fluorescently tagged structures were purified using ultrafiltration columns with a total of three wash steps with ultrapure water.

AGE analysis. The structures were analyzed using 0.3% agarose gels not pre-stained with DNA dye. The rest of the protocol was like the one mentioned earlier. The gel was scanned for Atto488 signal, followed by post-staining with SYBR safe DNA gel stain dye. For this, 120 mL of 0.5×TBE buffer was mixed with 12 μL of DNA stain. The gel was incubated in the staining solution for 30 min at room temperature. Prior to imaging, excess stain was removed by washing the gel with ultrapure water. This step was performed to reduce any background noise.

Preparation and Purification of DBCO or Biotin-Oligonucleotide-PEG Conjugates

Oligonucleotides with sequences complimentary to the single stranded extensions on the DNAO were obtained with amino terminal groups from IDT. In addition, DBCO-PEG-NHS, and Biotin-PEG-NHS, with 5 kDa PEG chain lengths were obtained from JenKem Technology USA.

Click chemistry between amino-oligo and NHS-PEG-DBCO or biotin. The reaction was performed in 50% DMSO, at pH 8.3 buffered using 1 M sodium bicarbonate solution. 25 times excess of NHS over amino was added to ensure high yields. Prior to the reaction the heterobifunctional PEG was dissolved in DMSO to prepare a 10 mM stock solution. The reaction was incubated overnight at room temperature. The resulting mixture was characterized by PAGE gel using the protocol mentioned above.

HPLC purification. For purification, the crude reaction mixture was lyophilized overnight. Next, to the lyophilized sample, 5 μl of acetonitrile (HPLC gradient grade, Sigma Aldrich) and 95 μL of 0.05 M, pH 7 Triethylammonium acetate (TEAA) buffer (Sigma Aldrich) were added and mixed thoroughly. The mixture was equilibrated for 30 min at room temperature and transferred to the HPLC vial. The solution was purified by reverse-phase high performance liquid chromatography (HPLC 1200 Agilent Technologies) with C3 column (Agilent Zorbax 300SB-C3) using a gradient of acetonitrile (concentration was increased from 5% to 50% over 30 min) in 0.05 mM TEAA (pH 7) at a flow rate of 0.4 mL/min as a mobile phase. The unreacted oligonucleotides were eluted at around 11-13 min, and PEG-modified oligonucleotides were eluted at around 20-22 min. The collected fractions were lyophilized and dissolved in 1×PBS for analysis with PAGE gel as mentioned above.

Validating DBCO activity after purification. To validate the activity of DBCO, homobifunctional PEG with Azido moiety were used to form dimers. The results were characterized using PAGE gels.

Adding purified oligo-PEG-DBCO or biotin to DNAO. The purified conjugates were added to the DNAO at ten times excess over each extension. The mixture was incubated for 1 hour at room temperature. The excess conjugates were removed using 50 kDa Pluronic passivated ultrafiltration columns.

Assembly of Iron Oxide Nanoparticles (IONPs) Functionalized Structures.

Purified DNAO, DOPMs or silica coated structures carrying 1, 2 or 3 binding sites with biotin groups were mixed with Streptavidin coated IONPs (Purchased from Ocean Nanotech, LLC USA) at three times excess over each binding site. The reaction mixture was incubated overnight at room temperature. The IONPs functionalized structures were immediately used for characterization TEM.

### LIST OF EMBODIMENTS

Embodiment 1. A construct, comprising: (a) a structured nucleic acid polymer micelle (DOPM), comprising: (i) a structured nucleic acid (DNAO) template; (ii) one or more functional moiety attached to the DNAO template; and (iii) polymers that interact with nucleic acid present in the DNAO template to form the DOPM; and (b) an inorganic shell surrounding the DNAO; wherein the one or more functional moiety extends outside the DOPM and the inorganic shell.

Embodiment 2. The construct of embodiment 1, wherein the DNAO template comprise DNA, RNA, LNA, or a combination thereof.

Embodiment 3. The construct of embodiment 1 or 2, wherein the DNAO template comprises a DNA origami template.

Embodiment 4. The construct of any one of embodiments 1-3, wherein the polymers comprise cationic polymers.

Embodiment 5. The construct of any one of embodiments 1-4, wherein the polymers comprise co-polymers comprising (a) a cationic polymer that electrostatically binds to negatively charged DNAO, and (b) a nonionic amphiphilic polymer.

Embodiment 6. The construct of embodiment 4 or 5, wherein the cationic polymers include, but are not limited to pLys, pHis, pArg, N,N-dimethylaminoethyl methacrylate (DMAEMA), poly amido amine (PAMAM), polyethyleneimine (PEI), cationic cyclodextrin, cationic cellulose, cationic dextrin, cationic dextran, chitosan, gelatin, spermine, spermidine, cationic surfactants such as CTAB or combination thereof.

Embodiment 7. The construct of embodiment 5 or 6, wherein the nonionic amphiphilic polymers include, but are not limited to, polyethylene glycol (PEG), polyoxazolines (POX), PVP (poly(N-vinylpyrrolidone), polyglycerols (PG), or combinations thereof.

Embodiment 8. The construct of any one of embodiments 1-7, wherein the polymers comprise co-polymers comprising (a) a cationic polymer that electrostatically binds to negatively charged DNAO, and (b) PEG.

Embodiment 9. The construct of embodiment 8 wherein the polymers comprise PEG-pLys, PEG-pArg, or PEG-pHis co-polymers, or combinations thereof.

Embodiment 10. The construct of any one of embodiments 7-9, wherein the PEG comprises PEG monomers of at least 2 KDa or at least 5 kDa in molecular weight.

Embodiment 11. The construct of any one of embodiments 1-10, wherein the polymers comprise a layer surrounding the DNAO.

Embodiment 12. The construct of embodiment 10, wherein the polymer layer has a thickness of between about 1 nm and about 20 nm.

Embodiment 13. The construct of any one of embodiments 1-12, wherein the DOPM comprises a polyplex micelle.

Embodiment 14. The construct of any one of embodiments 1-13, wherein the inorganic shell comprises a silica shell (SiO2), titania shell (TiO2), alumina shell (Al2O3), gold shell, silver shell, copper shell, iron shell, platinum shell, palladium shell, calcium phosphate shell, hydroxyapatite shell, or combinations thereof.

Embodiment 15. The construct of embodiment 14, wherein the inorganic shell comprises a silica shell.

Embodiment 16. The construct of embodiment 14 or 15, wherein the inorganic shell is between 1 nm and 20 nm in thickness.

Embodiment 17. The construct of embodiment 1, further comprising an inorganic core to which the structured nucleic acid template is bound.

Embodiment 18. The construct of embodiment 17, wherein the inorganic core comprises a magnetic core, a polymeric core, a metallic core, or a glass core.

Embodiment 19. The construct of embodiment 18, wherein the inorganic core is metallic and comprises a metal oxide, gold or silver.

Embodiment 20. The construct of embodiment 18, wherein the inorganic core is magnetic.

Embodiment 21. The construct of any one of embodiments 17-20, wherein the structured nucleic acid template covers about 10%, or about 20%, or about 30%, or about 40%, or about 50%, or about 60%, or about 70%, or about 80%, or about 90%, or about 100% of the surface of the inorganic core.

Embodiment 22. The construct of embodiment 21, wherein the structured nucleic acid template essentially surrounds the inorganic core.

Embodiment 23. The construct of any one of embodiments 1-16, comprising: (a) an outermost layer of a nonionic amphiphilic polymer component of the polymers and its terminal group (if any), (b) the inorganic shell layer below the outermost layer; and (c) an electrostatic complex of the DNAO and the cationic segment of the polymers surrounded by the inorganic shell.

Embodiment 24. The construct of any one of embodiments 1-23, wherein the polymers comprise PEG-pLys, PEG-pArg, or PEG-pHis co-polymers, or combinations thereof.

Embodiment 25. The construct of embodiment 17 or 18, wherein the inorganic shell layer comprises a silica shell layer.

Embodiment 26. The construct of any one of embodiments 1-25, wherein the one or more functional moiety is monofunctional.

Embodiment 27. The construct of any one of embodiments 1-25, wherein the one or more functional moiety is polyfunctional.

Embodiment 28. The construct of any one of embodiments 1-27, wherein the one or more functional moiety comprises one or more polymer.

Embodiment 29. The construct of any one of embodiments 1-28, wherein the one or more functional moiety comprises a linear PEG.

Embodiment 30. The construct of any one of embodiments 1-28, wherein the polymer comprises cationic polymer.

Embodiment 31. The construct of any one of embodiments 1-30, wherein the one or more functional moiety comprises an oligonucleotide that is complimentary to single-stranded nucleic acid extensions placed at one or more locations on the DNAO.

Embodiment 32. The construct of any one of embodiments 1-31, wherein the functional moiety comprises co-polymer comprising (a) a cationic polymer that electrostatically binds to negatively charged DNAO, and (b) a nonionic amphiphilic polymer.

Embodiment 33. The construct of any one of embodiments 1-32, comprising one or more functional moiety that comprises an oligonucleotide that is complimentary to single-stranded nucleic acid extensions placed at one or more locations on the DNAO, and one or more functional moiety that comprises co-polymer comprising (a) a cationic polymer that electrostatically binds to negatively charged DNAO, and (b) a nonionic amphiphilic polymer.

Embodiment 34. The construct of any one of embodiments 1-33, wherein the one or more functional moiety comprises a functional terminal group, including but not limited to biotin, streptavidin, dibenzocyclooctyne group (DBCO), azide, alkyne, amine, NHS esters (N-hydroxysuccinimide esters), thiol, maleimide, iodoacetyl, carboxyl, etc.

Embodiment 35. The construct of any one of embodiments 1-34, wherein the polymers that interact with nucleic acid present in the DNAO template to form the DOPM comprise: (a) a cationic polymer that electrostatically binds to negatively charged DNAO; (b) a nonionic amphiphilic polymer; and (c) a functional terminal group; and wherein a DNA template for amplification is bound to the one or more functional moiety attached to the DNAO template; and wherein a primer pair is bound to the functional terminal group on the polymer, the primer pair comprising: (i) a first primer sequence complementary to either a forward strand or a reverse strand of the DNA template; and (ii) a second primer sequence complementary to a DNA strand opposite the first primer sequence; wherein one primer in the primer pair comprises a cleavable linker.

Embodiment 36. The construct of embodiment 15, wherein the polymers that interact with nucleic acid present in the DNAO template to form the DOPM comprise: (a) a cationic polymer that electrostatically binds to negatively charged DNAO; (b) a nonionic amphiphilic polymer; and (c) a functional terminal group; and wherein a DNA template for amplification is bound to the one or more functional moiety attached to the DNAO template; and wherein a primer pair is bound to the functional terminal group on the polymer, the primer pair comprising: (i) a first primer sequence complementary to either a forward strand or a reverse strand of the DNA template; and (ii) a second primer sequence complementary to a DNA strand opposite the first primer sequence; wherein one primer in the primer pair comprises a cleavable linker.

Embodiment 37. The construct of any one of embodiments 1-36, wherein the DNAO is stabilized by crosslinking, such as by bi-functional molecules that covalently react with DNA strands constituting the DNAO.

Embodiment 38. The construct of any one of embodiments 1-37, wherein the construct is stable in non-aqueous, salt-free as well as organic solvents.

Embodiment 39. The construct of any one of embodiments 1-38, wherein the construct is stable in basic pH conditions and can sustain sudden pH changes.

Embodiment 40. The construct of any one of embodiments 1-39, wherein the construct is stable at 90° C. in salt-free buffer.

Embodiment 41. The construct of any one of embodiments 1-40, further comprising a cargo embedded in the organic shell, including but not limited to a therapeutic, diagnostic, detectable moiety, etc.

Embodiment 42. A monofunctional DNA origami nanoparticle conjugate construct, comprising: (a) an inorganic core; (b) one or more structured nucleic acid (DNAO) template attached to the core; (c) a binding functional moiety attached to the DNAO template for binding to a target analyte; and (d) one or more detection functional moiety attached to the DNAO template for detection of binding of a target analyte by the binding functional moiety.

Embodiment 43. The conjugate of embodiment 42, wherein the inorganic core comprises a magnetic core, a polymeric core, a metallic core, or a glass core.

Embodiment 44. The conjugate of embodiment 43, wherein the metallic inorganic core comprises an oxide of iron, gold or silver.

Embodiment 45. The conjugate of embodiment 44, wherein the inorganic core is magnetic.

Embodiment 46. The conjugate of any one of embodiments 42-45, wherein the structured nucleic acid template covers about 10%, or about 20%, or about 30%, or about 40%, or about 50%, or about 60%, or about 70%, or about 80%, or about 90%, or about 100% of the surface of the inorganic core.

Embodiment 47. The conjugate of embodiment 46, wherein the structured nucleic acid template essentially surrounds the inorganic core.

Embodiment 48. The conjugate of any one of embodiments 42-47, wherein the binding functional moiety is bound to a target analyte binding molecule.

Embodiment 49. The conjugate of embodiment 48, wherein the target analyte binding molecule comprises a binding protein, antibody, or aptamer.

Embodiment 50. The conjugate of embodiment 49, wherein the target analyte binding molecule comprises an antibody.

Embodiment 51. The conjugate of any one of embodiments 42-50, wherein the detection functional moiety comprises one or more of a fluorescent moiety, a redox moiety, an enzymatic reporter and a nucleic acid tag.

Embodiment 52. The conjugate of embodiment 51, wherein the detection functional moiety comprises a fluorescent moiety.

Embodiment 53. A composition, comprising a plurality (i.e., 2, 3, 4, 5, 6, 10, 25, 50, 100, 1000, 5000, 10000, 100000, 1000000, or more) constructs of any one of embodiments 1-52.

Embodiment 54. The composition of embodiment 53, wherein the plurality of constructs are monodispersed.

Embodiment 55. The composition of embodiment 53 or 54, wherein the composition is in solution.

Embodiment 56. The composition of embodiment 55, wherein the solution comprises an organic solvent.

Embodiment 57. A method of making the construct of any one of embodiments 1-41, comprising: (a) providing a DNAO template having one or more functional moiety attached to the DNAO; (b) binding the DNAO with polymers that interact with nucleic acid present in the DNAO template to form the DOPM; and (c) encasing the DNAO in an inorganic shell.

Embodiment 58. A method of making the construct of embodiment 35, comprising: (a) providing a DNAO template having one or more functional moiety attached to the DNAO, the functional moiety comprising an oligonucleotide that is complimentary to single-stranded nucleic acid extensions placed at one or more locations on the DNAO, a polymer spacer, and a functional terminal group; (b) binding the DNAO with co-polymers comprising: (i) a cationic polymer that electrostatically binds to negatively charged DNAO, (ii) a nonionic amphiphilic polymer; and (iii) a functional terminal group bound to the nonionic amphiphilic polymer opposite to the bound cationic polymer that interact with nucleic acid present in the DNAO template to form the DOPM; (c) encasing the DNAO in an inorganic shell; (d) binding at least one DNA template to a functional terminal group bound to a functional moiety complimentary to single-stranded nucleic acid extensions placed at one or more locations on the DNAO; and (e) binding at least one primer pair to a functional terminal group bound to a functional moiety comprising a co-polymer.

Embodiment 59. A method of making the construct of embodiment 35, comprising (a) providing a DNAO template having one or more functional moiety attached to the DNAO, the functional moiety comprising an oligonucleotide that is complimentary to single-stranded nucleic acid extensions placed at one or more locations on the DNAO, a polymer spacer, and a functional terminal group; (b) binding the DNAO with co-polymers comprising: (i) a cationic polymer that electrostatically binds to negatively charged DNAO, (ii) a nonionic amphiphilic polymer; and (iii) a functional terminal group bound to the nonionic amphiphilic polymer opposite to the bound cationic polymer that interact with nucleic acid present in the DNAO template to form the DOPM; (c) encasing the DNAO in an inorganic shell; (d) binding at least one DNA template to a first functional terminal group bound to a functional moiety complimentary to single-stranded nucleic acid extensions placed at one or more locations on the DNAO; (e) binding at least one primer pair to a functional terminal group bound to a functional moiety comprising a co-polymer; (f) binding the construct to a surface through a second functional terminal group on a second functional moiety that also comprises an oligonucleotide that is complimentary to single-stranded nucleic acid extensions placed at one or more locations on the DNAO.

Embodiment 60. The method of any one of embodiments 57-59, wherein the polymers comprise polymers as recited in any one of embodiments 4-10.

Embodiment 61. The method of any one of embodiments 57-60, wherein the polymers comprise co-polymers comprising (a) a cationic polymer that electrostatically binds to negatively charged DNAO, and (b) PEG.

Embodiment 62. The method of embodiment 61 wherein the polymers comprise PEG-pLys, PEG-pArg, or PEG-pHis co-polymers, or combinations thereof.

Embodiment 63. The method of any one of embodiments 61 or 62, wherein the PEG comprises PEG monomers of at least 2 KDa or at least 5 kDa in molecular weight.

Embodiment 64. The method of any one of embodiments 57-63, wherein encasing the DNAO in an inorganic shell comprises combining the DOPM with an inorganic molecule precursor in in an aqueous alcohol solution to form the inorganic shell encasing the DNAO.

Embodiment 65. The method of embodiment 64, wherein the combining comprises combining the DOPM with an inorganic molecule precursor and a reducing agent in in an aqueous alcohol solution to form the inorganic shell encasing the DNAO.

Embodiment 66. The method of embodiment 64 or 65, wherein the inorganic shell comprises a silica shell.

Embodiment 67. The method of embodiment 66, wherein the inorganic molecule precursor comprises tetramethyl orthosilicate (TMOS), tetraethyl orthosilicate (TEOS), or combinations thereof.

Embodiment 68. The method of embodiment 66 or 67, wherein the aqueous alcohol solution comprises aqueous ethanol.

Embodiment 69. The method of any one of embodiments 66-68, wherein the aqueous solution is at about pH 11.

Embodiment 70. The method of any one of embodiments 66-69, wherein encasing the DNAO in an inorganic shell is carried out at a temperature of between about 4° C. and about 45° C., optionally at about room temperature (about 23° C.).

Embodiment 71. The method of embodiment 64 or 65, wherein the inorganic shell comprises a gold shell.

Embodiment 72. The method of embodiment 71, wherein the inorganic molecule precursor comprises hydrochloric acid (H[AuCl4]), gold (iii) acetate (Au(C₂H₃O₂)3), or combinations thereof.

Embodiment 73. The method of embodiment 71 or 72, wherein method further the reducing agent comprises sodium citrate, formaldehyde, sodium borohydride, or combinations thereof.

Embodiment 74. The method of any one of embodiments 71-73, wherein encasing the DNAO in an inorganic shell is carried out at a temperature of between about 4° C. and about 45° C., optionally at about room temperature (about 23° C.).

Embodiment 75. The method of embodiment 64 or 65, wherein the inorganic shell comprises a silver shell.

Embodiment 76. The method of embodiment 75, wherein the inorganic molecule precursor comprises silver nitrate.

Embodiment 77. The method of embodiment 75 or 76, wherein the reducing agent comprises sodium citrate, formaldehyde, sodium borohydride, or combinations thereof.

Embodiment 78. The method of any one of embodiments 75-77, wherein encasing the DNAO in an inorganic shell is carried out at a temperature of between about 4° C. and about 45° C., optionally at about room temperature (about 23° C.).

Embodiment 79. The method of embodiment 64 or 65, wherein the inorganic shell comprises an iron shell.

Embodiment 80. The method of embodiment 79, wherein the inorganic molecule precursor comprises ferric chloride (FeCl2) and ferrous chloride (FeCl3).

Embodiment 81. The method of embodiment 62 or 63 79 or 80, wherein the reducing agent comprises sodium citrate, formaldehyde, sodium borohydride, or combinations thereof.

Embodiment 82. The method of any one of embodiments 79-81, wherein encasing the DNAO in an inorganic shell is carried out at a temperature of between about 4° C. and about 45° C., optionally at about room temperature (about 23° C.).

Embodiment 83. The method of embodiment 64 or 65, wherein the inorganic shell comprises a calcium phosphate shell.

Embodiment 84. The method of embodiment 83, wherein the inorganic molecule precursor comprises calcium chloride and sodium phosphate dibasic dodecahydrate.

Embodiment 85. The method of any one of embodiments 83 or 84, wherein the aqueous solution is at about pH 9.

Embodiment 86. The method of any one of embodiments 83-85, wherein encasing the DNAO in an inorganic shell is carried out at a temperature of between about 4° C. and about 45° C., optionally at about room temperature (about 37° C.).

Embodiment 87. The method of embodiment 59, wherein the surface is in a flow cell.

Embodiment 88. The method of embodiment 87, wherein the flow cell is patterned or unpatterned.

Embodiment 89. A method for making a monoclonal cluster of a target oligonucleotides on a particle comprising: (a) providing a construct according to embodiment 35 or clam 36; (b) binding a DNA template to a first functional terminal group on one or more functional moiety that binds to single-stranded nucleic acid extensions placed at one or more locations on the DNAO; (c) binding a primer pair to a functional terminal group on the one or more co-polymer functional moiety, wherein the primer pair comprises a first primer sequence and a second primer sequence, the first primer sequence being complementary to either a forward strand or a reverse strand of the DNA template, and the second primer sequence being complementary to a DNA strand opposite the first primer sequence; and one primer in the primer pair comprises a cleavable linker; (d) amplifying the DNA template on the surface of the construct by bridge amplification; (e) cleaving the cleavable linker.

Embodiment 90. The method of embodiment 89, wherein the amplification is performed in solution.

Embodiment 91. The method of embodiment 89, further comprising binding the construct to a surface through a second functional terminal group on one or more functional moiety that comprises an oligonucleotide that is complimentary to single-stranded nucleic acid extensions placed at one or more locations on the DNAO.

Embodiment 92. The method of embodiment 91, wherein the surface is in a flow cell.

Embodiment 93. The method of embodiment 92, wherein the flow cell is patterned or unpatterned.

Embodiment 94. A method for making a monofunctional DNA origami nanoparticle conjugate construct, comprising: (a) providing an inorganic core; (b) binding one or more DNA templates to the inorganic core; (c) providing a binding functional moiety for the DNA template to bind to a target analyte; and (d) providing one or more detection functional moieties for the DNA template for detection of binding of a target analyte by the binding functional moiety; (e) providing one or more staple strands; (f) providing buffer conditions amenable to self-assembly of the DNA template(s) into structured DNAO.

Embodiment 95. The method of embodiment 94, wherein the DNA template is unstructured when bound to the inorganic core.

Embodiment 96. The method of embodiment 95, wherein the binding functional moiety and the one or more detection functional moieties are bound to the unstructured DNA template before addition of the staple strands.

Embodiment 97. The method of embodiment 94, wherein the binding functional moiety, and the one or more detection functional moieties comprise a staple strand sequence for incorporation into specific locations on the structured DNAO.

Embodiment 98. The method of embodiment 94, wherein steps (c) through (f) precede steps (a) and (b) so that a structured DNAO is formed prior to binding to the inorganic core.

Embodiment 99. The method of embodiment 94, wherein the one or more structured DNAO bound to the inorganic core covers about 10%, or about 20%, or about 30%, or about 40%, or about 50%, or about 60%, or about 70%, or about 80%, or about 90%, or about 100% of the surface of the inorganic core.

Embodiment 100. The method of embodiment 94, wherein the binding functional moiety comprises a binding protein, antibody, or aptamer.

Embodiment 101. The method of embodiment 100, wherein the binding functional moiety comprises an antibody.

Embodiment 102. The method of embodiment 94, wherein the detection functional moiety emits a detectable signal.

Embodiment 103. The method of embodiment 103, wherein the detectable signal comprises one or more of fluorescence, redox state, an enzymatic reporter, or nucleic acid tags.

Embodiment 104. The method of embodiment 101, wherein the detection functional moiety comprises one or more of a fluorescent moiety, a redox moiety, an enzymatic reporter and a nucleic acid tag.

1. A construct, comprising:
(a) a structured nucleic acid polymer micelle (DOPM), comprising:
(i) a structured nucleic acid (DNAO) template;
(ii) one or more functional moiety attached to the DNAO template; and
(iii) polymers that interact with nucleic acid present in the DNAO template to form the DOPM; and
(b) an inorganic shell surrounding the DOPM;
wherein the one or more functional moiety extends outside the DOPM and the inorganic shell.

2. The construct of claim 1, wherein the inorganic shell comprises silica.

3. The construct claim 1, wherein the structured nucleic acid (DNAO) template comprises DNA, wherein the polymers comprise one or more co-polymers comprising (a) a cationic polymer that electrostatically binds to negatively charged DNAO, and (b) a nonionic amphiphilic polymer; and wherein structured nucleic acid (DNAO) template.

4. The construct of claim 3, wherein the cationic polymers comprise one or more of pLys, pHis, pArg, N,N-dimethylaminoethyl methacrylate (DMAEMA), poly amido amine (PAMAM), polyethyleneimine (PEI), cationic cyclodextrin, cationic cellulose, cationic dextrin, cationic dextran, chitosan, gelatin, spermine, spermidine, cationic surfactants, or combination thereof, and wherein the nonionic amphiphilic polymers comprise polyethylene glycol (PEG), polyoxazolines (POX), PVP (poly(N-vinylpyrrolidone), polyglycerols (PG), or combinations thereof.

5. The construct of claim 1, further comprising an inorganic core to which the structured nucleic acid template is bound.

6. The construct of claim 1, comprising one or more functional moiety that comprises an oligonucleotide that is complimentary to single-stranded nucleic acid extensions placed at one or more locations on the DNAO, and one or more functional moiety that comprises co-polymer comprising (a) a cationic polymer that electrostatically binds to negatively charged DNAO, and (b) a nonionic amphiphilic polymer.

7. The construct of claim 1, wherein the one or more functional moiety comprises a functional terminal group comprising one or more of biotin, streptavidin, dibenzocyclooctyne group (DBCO), an azide, an alkyne, an amine, an NHS ester (N-hydroxysuccinimide esters), a thiol, a maleimide, iodoacetyl, or a carboxyl.

8. The construct of claim 1, wherein the polymers that interact with nucleic acid present in the DNAO template to form the DOPM comprise:
(a) a cationic polymer that electrostatically binds to negatively charged DNAO;
(b) a nonionic amphiphilic polymer; and
(c) a functional terminal group; and
wherein a DNA template for amplification is bound to the one or more functional moiety attached to the DNAO template; and
wherein a primer pair is bound to the functional terminal group on the polymer, the primer pair comprising:
(i) a first primer sequence complementary to either a forward strand or a reverse strand of the DNA template; and
(ii) a second primer sequence complementary to a DNA strand opposite the first primer sequence;
wherein one primer in the primer pair comprises a cleavable linker.

9. The construct of claim 1, wherein the DNAO is stabilized by crosslinking, such as by bi-functional molecules that covalently react with DNA strands constituting the DNAO.

10. A monofunctional DNA origami nanoparticle conjugate construct, comprising:
(a) an inorganic core;
(b) one or more structured nucleic acid (DNAO) template attached to the core;
(c) a binding functional moiety attached to the DNAO template for binding to a target analyte; and
(d) one or more detection functional moiety attached to the DNAO template for detection of binding of a target analyte by the binding functional moiety.

11. The conjugate of claim 10, wherein the inorganic core comprises a magnetic core, a polymeric core, a metallic core, or a glass core.

12. The conjugate of claim 10, wherein the target analyte binding functional moiety comprises a binding protein, antibody, or aptamer.

13. The conjugate of claim 10, wherein the detection functional moiety comprises one or more of a fluorescent moiety, a redox moiety, an enzymatic reporter and a nucleic acid tag.

14. A composition, comprising a plurality of constructs of claim 1.

15. A method of making the construct of claim 1, comprising
(a) providing a DNAO template having one or more functional moiety attached to the DNAO;
(b) binding the DNAO with polymers that interact with nucleic acid present in the DNAO template to form the DOPM; and
(c) encasing the DNAO in an inorganic shell.

16. A method of making the construct of claim 8, comprising
(a) providing a DNAO template having one or more functional moiety attached to the DNAO, the functional moiety comprising an oligonucleotide that is complimentary to single-stranded nucleic acid extensions placed at one or more locations on the DNAO, a polymer spacer, and a functional terminal group;
(b) binding the DNAO with co-polymers comprising:
(i) a cationic polymer that electrostatically binds to negatively charged DNAO,
(ii) a nonionic amphiphilic polymer; and
(iii) a functional terminal group bound to the nonionic amphiphilic polymer opposite to the bound cationic polymer that interact with nucleic acid present in the DNAO template to form the DOPM;
(c) encasing the DNAO in an inorganic shell;
(d) binding at least one DNA template to a functional terminal group bound to a functional moiety complimentary to single-stranded nucleic acid extensions placed at one or more locations on the DNAO; and
(e) binding at least one primer pair to a functional terminal group bound to a functional moiety comprising a co-polymer.

17. A method for making a monoclonal cluster of a target oligonucleotides on a particle comprising
(a) providing a construct according to claim 8;
(b) binding a DNA template to a first functional terminal group on one or more functional moiety that binds to single-stranded nucleic acid extensions placed at one or more locations on the DNAO;
(c) binding a primer pair to a functional terminal group on the one or more co-polymer functional moiety, wherein the primer pair comprises a first primer sequence and a second primer sequence, the first primer sequence being complementary to either a forward strand or a reverse strand of the DNA template, and the second primer sequence being complementary to a DNA strand opposite the first primer sequence; and one primer in the primer pair comprises a cleavable linker;
(d) amplifying the DNA template on the surface of the construct by bridge amplification;
(e) cleaving the cleavable linker.

18. A method for making a monofunctional DNA origami nanoparticle conjugate construct, comprising:
(a) providing an inorganic core;
(b) binding one or more DNA templates to the inorganic core;
(c) providing a binding functional moiety for the DNA template to bind to a target analyte; and
(d) providing one or more detection functional moieties for the DNA template for detection of binding of a target analyte by the binding functional moiety;
(e) providing one or more staple strands;
(f) providing buffer conditions amenable to self-assembly of the DNA template(s) into structured DNAO.

19. The method of claim 18, wherein steps (c) through (f) precede steps (a) and (b) so that a structured DNAO is formed prior to binding to the inorganic core.

20. The method of claim 18, wherein the binding functional moiety comprises a binding protein, antibody, or aptamer.


SEQUENCE LISTING (2 sequences)

SEQ ID NO: 1 - DNA, 20 bp, synthetic construct
  source 1..20: mol_type = other DNA
caacctcataccactacaac

SEQ ID NO: 2 - DNA, 20 bp, synthetic construct
  source 1..20: mol_type = other DNA
gttgtagtggtatgaggttg